(19) United States
(12) Patent Application Publication
(10) Pub. No.: US 2026/0235615 A1
(43) Pub. Date: Aug. 13, 2026

(54) PROTEIN / POLYPEPTIDE SEQUENCING BY EXPANSION (PROSE)

(71) Applicant: Oregon Health & Science University, Portland, OR (US)

(72) Inventors: Eugene Tu, Portland, OR (US); Justin Stanley Plaut, Portland, OR (US); Nicholas Scott Groves, Portland, OR (US); Yu-Jui Chiu, Portland, OR (US); Andy Mendoza, Portland, OR (US); Riccardo Carloni, Portland, OR (US); Srivathsan Ranganathan, Portland, OR (US)

(73) Assignee: Oregon Health & Science University, Portland, OR (US)

(21) Appl. No.: 19/471,678

(22) PCT Filed: Apr. 2, 2024
(86) PCT No.: PCT/US2024/022652

§ 371 Date: Oct. 1, 2025

Related U.S. Application Data
(60) Provisional application No. 63/493,769, filed on Apr. 2, 2023.

(51) Int. Cl. G01N 33/68; C07H 21/02; C07H 21/04; C07K 1/107

(52) U.S. Cl. CPC G01N 33/6824; C07H 21/02; C07H 21/04; C07K 1/1077

(57) ABSTRACT

Described herein are methods and systems useful to analyze proteins/polypeptides, including sequentially sequencing individual polypeptides, as well as compounds useful in carrying out such methods. Such compounds include those depicted in Formulas (I)-(XX). In embodiments, the methods involve expanding the distance between each amino acid of a polypeptide chain by transferring individual amino acids, in order, from a donor polypeptide to a polymer (e.g., a nucleic acid backbone) to form a hybrid polymer (a PROSE construct), then sequencing the individual amino acids along the PROSE construct. Such sequencing may involve nanopore-type sequencing, SMRT-sequencing, and the like.

### CROSS-REFERENCE TO RELATED APPLICATION

This is the 371 National Phase of International Application No. PCT/US2024/022652, filed Apr. 2, 2024, which claims priority to and the benefit of the earlier filing of U.S. Provisional Application No. 63/493,769, filed on Apr. 2, 2023, which is incorporated by reference herein in its entirety.

### INCORPORATION BY REFERENCE OF SEQUENCE LISTING

A computer readable file, entitled “0046-0073US_SeqList.xml” created on or about Oct. 1, 2025, with a file size of 110,592 bytes, contains the sequence listing for this application and is hereby incorporated by reference in its entirety.

### FIELD OF THE DISCLOSURE

The present disclosure relates generally to sequencing of a polypeptide or protein via a single molecule approach, such as nanopores.

### BACKGROUND OF THE DISCLOSURE

The flow of genetic information in cells can be described by three fundamental transformations: DNA to DNA (replication), DNA to RNA (transcription) and RNA to protein (translation). Regarding functionality, DNA and RNA determine the structure of proteins but the ultimate function (or dysfunction in a disease) of a cell is determined by proteins expressed in the cells. Considering the advanced understanding of various diseases enabled by modern nucleic acid sequencing methods, it stands to reason that de novo peptide methods and protein profiling could have a similarly profound effect. Therefore, de novo peptide sequencing platforms are needed in disease research for discovering differentiating factors in disease progression as well as novel disease biomarkers.

State of De Novo Peptide Sequencing: Currently, untargeted proteomics primarily relies on digesting intact proteins into small peptides, measuring their mass to charge ratios (m/z) with liquid chromatography-mass spectrometry (LC-MS), and then mapping this data back to reference databases of known proteins. For this reason, despite being the gold standard for peptide sequencing, LC-MS does not technically sequence the peptide, but rather attempts to assign sequence identities to each peptide based on prior knowledge of the proteome. LC-MS exhibits other limitations including a high technical barrier to entry, high capital and sample costs, low dynamic range, incomplete proteome coverage, and difficulty assigning identities to peptide sequences with similar m/z ratios. Recently, several de novo peptide sequencing technologies and companies have emerged with considerable financial backing. These technologies can be broadly separated into two categories: those that rely on affinity agents and those that do not. As demonstrated by the following examples, these various peptide sequencing technologies require significant further technological, chemical, and methodological development to achieve true de novo sequencing.

One variation of affinity agent-based technologies uses specific N-terminal amino acid (NTAA) specific binders for amino acid identification. Encodia, Quantum-Si, and Glyphic Bio are companies that use NTAA binders to aid in peptide sequencing, albeit with different workflows and readout types (Reed et al., Science 378(6616):186-192, 2022. doi.org/10.1126/science.abo7651; US20200231956A1). These methods are promising, but finding specific binders for each amino acid is extremely challenging. Thus, generating a library of against all or most of the canonical amino acids is currently infeasible and only sparse peptide sequencing can be achieved (that is, only certain amino acids within a peptide can be identified). Due to the difficulty of developing specific binders, these technologies also have limited capability to identify post-translational modifications (PTMs). A second variation of affinity agent-based technologies uses specific binders to identify whole proteins. Intact protein profiling has been attempted by Nautilus with some success, but this method relies on a broad affinity reagent library that does not presently exist (see U.S. Pat. No. 11,545,234).

One non-affinity agent approach was developed by Erisyon, which uses covalent dye attachment to amino acids for identification (see U.S. Pat. No. 11,105,812). However, this technology is limited to a subset of amino acids with reactive side chains and, thus, can also only achieve sparse sequencing with limited capability to identify PTMs.

A second non-affinity agent approach was developed by Dreampore, which uses a nanopore-based approach to identify amino acids. However, this approach of passing an intact peptide through a nanopore to identify amino acids has only been demonstrated with contrived peptides (Ouldali et al., Nat Biotechnol 38(2):176-181, 2020. doi.org/10.1038/s41587-019-0345-2). When an intact peptide passes through a nanopore, numerous amino acids within the chain contribute to the measured electrical signal. Due to the extreme complexity of this convoluted signal, this approach cannot feasibly sequence intact peptides of unknown sequence using existing technology.

Nanopore Sequencing: Technologies to sequence poly-nucleotides has been implemented by various entities, including Oxford Nanopore Technologies and Roche. Generally, nanopore sequencing relies on passing molecules, such as biopolymers, through either a solid-state or naturally derived pore with angstrom- to nanometer-scale internal features. As the molecule passes through the pore, a signal corresponding to the size and chemical properties of the molecule's structure is acquired. This signal is often an electrical readout such as a continuous measurement of current with respect to time. From this data, the molecule's rate of translocation through the pore can be determined (Wang et al., Nat Biotechnol 39(11):1348-1365, 2021. doi.org/10.1038/s41587-021-01 108-x; Caldwell et al., Proc Natl Acad Sci USA 114(45):11809-11811, 2017. doi.org/10.1073/pnas.1716866114).

Currently, naturally derived biological pores composed of proteins are most commonly used. These pores include derivatives of α-hemolysin, porin A, and CsgG (Caldwell et al., Proc Natl Acad Sci USA 114(45):11809-11811, 2017. doi.org/10.1073/pnas.1716866114). While solid-state pores are an area of intense research, there are several extant problems that limit their current viability, including poor chemical specificity, unstable pore size, and imprecise manufacturing (Chen et al., Sensors (Basel) 19(8):2019. doi.org/10.3390/s19081886).

To generate a readout, the molecule must be passed through the nanopore. There are multiple means of translocating a molecule through a pore including manipulation with physical fields (for example, application of a magnetic field after attachment to a magnetic particle) or the action of molecule motors. Commonly used molecular motors are naturally derived proteins that can processively translocate biopolymers through a nanopore. These include derivatives of translocases, nucleic acid polymerases, and helicases, such as Phi29 DNA polymerase and helicase Hel308 (Caldwell et al., Proc Natl Acad Sci USA 114(45):11809-11811, 2017. doi.org/10.1073/pnas.1716866114).

As a polymer passes through a nanopore, multiple monomeric units of the polymer are contained within the geometry of the pore and simultaneously contribute to the readout to form a convoluted signal. Therefore, instead of acquiring sequence-specific data from single monomers of polymers, the signal is measured from a subsequence of the polymer (“k”), which typically contains several monomeric units. Therefore, a unique signal can be acquired from each permutation of sequences of length k. Since sequence permutations increase exponentially, increasing k lengths can rapidly grow the set of possible subsequences. The k length can be reduced through nanopore engineering. One approach focusses on designing one or more choke points (“read heads”) within the interior of the pore to enhance the signal contributed by a shorter length of polymer.

For poly-nucleotides, the Oxford Nanopore Technologies' CsgG-derived R9 nanopore has a k of 5 to 6 nucleotides. That is, the subsequence of 5 to 6 nucleotides occupying a narrowed region of the pore account for most of the measured changes in electrical current (Wang et al., Nat Biotechnol 39(11):1348-1365, 2021. doi.org/10.1038/s41587-021-01108-x; Jain et al., Genome Biol 17(1):239, 2016. doi.org/10.1186/s13059-016-1103-0). The measured signal from known poly-nucleotide sequences can be used to train a machine learning algorithm to recognize all N^{k} permutations of the N=4 different canonical nucleotides for RNA or DNA. For k=6, there are 4096 possible permutations of canonical nucleotides, each of which may produce a characteristic signal as it passes through the pore. The main sources of sequencing error are homopolymeric sequences with lengths greater than the occupancy of the read head.

In pores with larger regions of base sensitivity, due to the presence of multiple and/or larger read heads, the signal complexity can be too great to deconvolve the signal and assign sequences at single-nucleotide resolution. For instance, for a pore with k=12, such as α-hemolysin, the number of possible sequence permutations N^{k} expands to 1.68×10⁷. It is neither technically nor practically viable to train a model to accurately identify each of these nearly 17 million sequences because limitations in signal resolution and extreme computational demand will preclude unambiguous single-nucleotide sequencing (Stoddart et al., Angew Chem Int Ed Eng/49(3):556-9, 2010. doi.org/10.1002/anie.200905483).

Nanopore-based peptide sequencing presents a similar challenge since peptides are composed of up to 21 different amino acids and each monomer of the peptide backbone is shorter in length than each monomer of the poly-nucleotide backbone. Therefore, both N and k are larger for peptides than for poly-nucleotides. Using published backbone contour lengths for peptides (0.40 nm per amino acid; Ainavarapu et al., Biophys J 92(1):225-33, 2007. doi.org/10.1529/biophysj.106.091561) and single-stranded DNA (0.68 nm per base pair; Chi et al., Physica A: Statistical Mechanics and its Applications 392(5):1072-1079, 2013. doi.org/10.1016/j.physa.2012.09.022), N=21 amino acids, k=6 DNA nucleotide lengths, and ignoring the smaller cross-section of peptides compared to single-stranded DNA (which could introduce further signal variability during nanopore translocation), the number of permutations N^{k}that could each have distinct signals burgeons to 1.92×10¹³. Even if selenocysteine and the length differences between peptides and single-stranded DNA are ignored, then the number of sequence permutations would be 6.4×10⁷. These estimates also ignore the broad variety of amino acid post-translational modifications that, if included, would drastically increase the possible sequence diversity. As with the example of the k=12 nucleotide nanopore, it is neither technically nor practically viable to accurately assign sequences to each of these sequence permutations at single-amino acid resolution. Therefore, due to this occupancy problem, which is exacerbated by the substantial chemical diversity and small size of amino acids, the direct de novo sequencing of intact peptides using nanopores is not currently possible.

### SUMMARY OF THE DISCLOSURE

Described herein are methods of de novo peptide sequencing method via translocation through a nanopore that are enabled by a novel polymeric construct (FIG. 1). Herein, the method is referred to as PROtein Sequencing by Expansion (“PROSE”) and the biochemical construct is referred to as a “PROSE construct”. Within the PROSE construct, the identity and relative positioning of each amino acid side chain of a peptide of arbitrary sequence is encoded albeit with expanded, user-defined spacing between adjacent residues. Side chains are encoded within the construct via a cyclic process. First, a polymeric seed block (“X”) is conjugated to a peptide. Next, a process involving the conjugation of a polymeric block to the N-terminal amino acid (NTAA) of a peptide, conjugation or ligation of this polymeric block to X (first cycle) or the preceding block (subsequent cycles), and cleavage of the NTAA to liberate an amino acid-bearing block. This is repeated until the desired number of amino acids of a peptide is encoded into the PROSE construct (FIG. 1). If applied to either the full length or component peptides of an unknown protein or mixture of proteins, then these unknown sequences can be encoded within multiple PROSE constructs for subsequent nanopore sequencing and proteome alignment. PROSE constructs can also be concatenated together to take advantage of nanopore long read lengths, which can be as high as 4 megabases (Mb, where 1 Mb is 1×10⁶ bases). Individual PROSE constructs can be identified by a unique molecular identifier (UMI) or barcode, which can be composed of DNA, amino acids, or unique chemical motifs. Due to the spatial separation of each amino acid side chain within the construct, the signal from each encoded acid is unambiguously detected during nanopore translocation, which represents a significant simplification of the occupancy problem exhibited by unexpanded peptides. Furthermore, this method is agnostic to the identity of the side chain present, so it is capable of detecting canonical side chains as well as post-translational, unnatural, and synthetic modifications of these side chains without any change to the process. Using a machine learning algorithm, the identities of each amino acid within the donor peptides can be assigned with positional accuracy to achieve single-amino acid resolution de novo peptide sequencing can be achieved.

The current disclosure provides a hybrid polymer including at least one repeat unit having the structure of any one of Formulae (I)-(X):

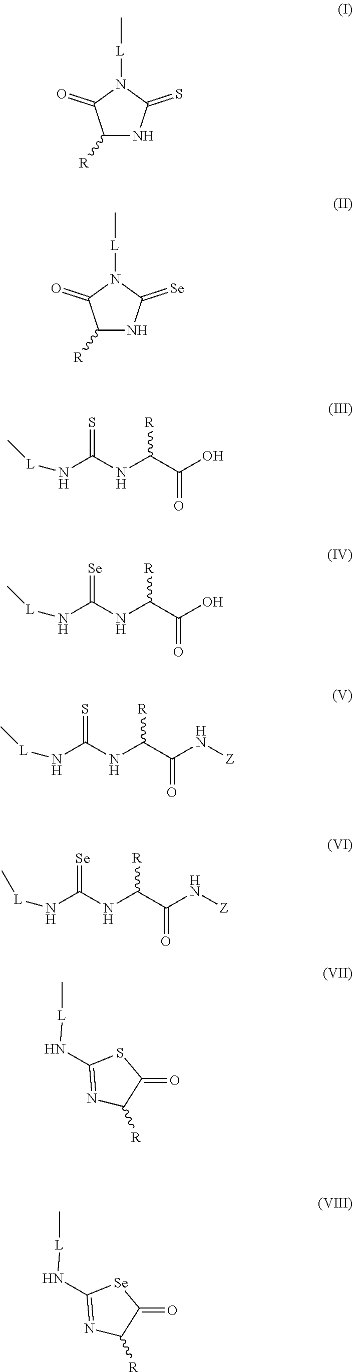

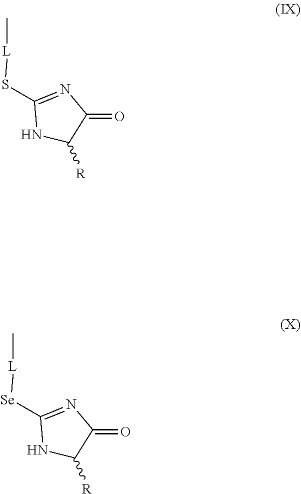

wherein the symbol “ [symbol] ” represents a single-stranded natural or synthetic biopolymer; each R independently is the side chain, protected side chain or modified side chain of a (D)- or (L)-alpha amino acid; L is a linker group; [symbol] indicates that the amino acid could be a (D)- or an (L)-alpha amino acid; and each “Z” can be a hydrogen atom, single amino acid, peptide of 2 to 200 amino acids, or a protein of up to 2000 amino acids. The amino acid(s) can be natural, unnatural, or synthetic. As used herein, the linker group L links the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein. L is the structure resulting from a reactive functionality on the AB or hybrid polymer and a reactive functionality on the NTAA of the peptide or resultant SCD. The structure includes any structures or spacers included in the creation of the reactive functionalities.

The current disclosure provides a hybrid polymer including at least one repeat unit having the structure of any one of Formulae (XI)-(XX):

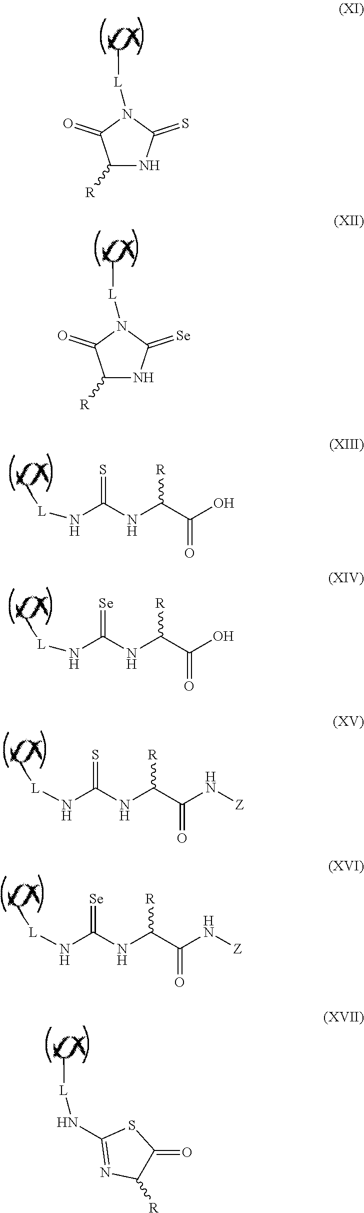

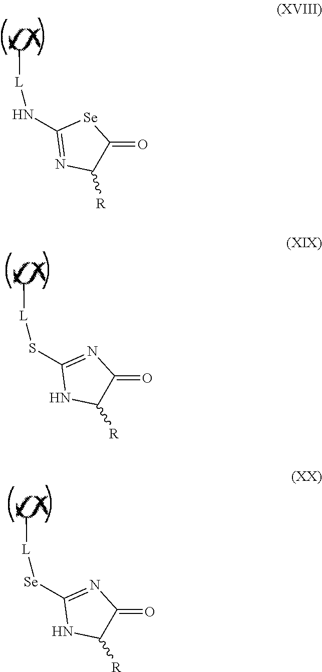

wherein the symbol “ [symbol] ” represents a double stranded natural or synthetic biopolymer; each R independently is the side chain, protected side chain or modified side chain of a (D)- or (L)-alpha amino acid; L is a linker group; [symbol] indicates that the amino acid could be a (D)- or an (L)-alpha amino acid; and each “Z” can be hydrogen, a single amino acid, a peptide of about 2 to about 200 amino acids, or a protein of up to 2000 amino acids. The amino acid(s) can be natural, unnatural, or synthetic. As used herein, the linker group L links the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein. L is the structure resulting from a reactive functionality on the AB or hybrid polymer and a reactive functionality on the NTAA of the peptide or resultant SCD. The structure includes any structures or spacers included in the creation of the reactive functionalities.

The current disclosure also provides a method of making a hybrid polymer (which may be referred to herein as a PROSE construct), including: modifying a donor polypeptide having a C-terminal amino acid (CTAA) and an N-terminal amino acid (NTAA), such that in the modification, the adjacent amino acids of the donor peptide are separated by segments of a polymeric seed strand (PSS) having a distal end and a proximal end, and wherein in the hybrid polymer the identify and relative position of each amino acid of the donor polypeptide is retained.

In embodiments, in the hybrid polymer, the protection or the modification of the side chain is to minimize or prevent unwanted side reactions or to render the side chain more detectable than the unprotected or unmodified side chain.

In embodiments, in the hybrid polymer, the natural or synthetic biopolymer includes repeat units of nucleic acids and/or amino acids and/or hydrocarbons.

In embodiments, in the hybrid polymer, the natural biopolymer includes a polynucleotide. In particular embodiments, the polynucleotide includes a single-stranded DNA or RNA. In examples of such embodiments, the hybrid polymer includes: a mixture of each repeat unit represented by Formula (I), Formula (II), Formula (III), and/or Formula (IV), Formula (IX) and/or Formula (X); a mixture of each repeat unit represented by Formula (I), Formula (II), Formula (III), and/or Formula (IV), Formula (IX) and/or Formula (X); a mixture of each repeat unit represented by Formula (I), Formula (II), Formula (III), and/or Formula (IV); a mixture of each repeat unit represented by Formula (IX), and/or Formula (X); a mixture of each repeat unit represented by Formula (I) and Formula (III); or each repeat unit is represented by Formula (IX).

In embodiments, in the hybrid polymer, the polynucleotide includes a double-stranded DNA or RNA. In examples of such embodiments, the hybrid polymer includes: a mixture of each repeat unit represented by Formula (XI), Formula (XII), Formula (XIII), Formula (XIV), Formula (XIX), and/or Formula (XX); a mixture of each repeat unit represented by Formula (XI), Formula (XII), Formula (XIII), and/or Formula (XIV); a mixture of each repeat unit represented by Formula (XIX), and/or Formula (XX); a mixture of each repeat unit represented by Formula (XI) and Formula (XIII); each repeat unit represented by Formula (XIX); or a mixture of each repeat unit represented by Formula (I) and/or Formula (IX).

In embodiments, in the method of making the hybrid polymer, the modifying includes: (a) attaching the distal end of the PSS to a docking linker (DL) that connects the C-CTAA of the donor polypeptide and the PSS, and wherein the other end of the polymeric seed strand is the proximal end; (b) attaching an assembly block (AB) to the NTAA of the donor polypeptide by reaction between a reactive functionality in the NTAA and a reactive functionality in the AB; (c) ligating the AB to the proximal end of the polymeric seed strand; (d) cleaving the bond between the NTAA that is attached to the AB and the prior amino acid attached to the NTAA to expose a new NTAA; and (e) repeating steps (b) through (d) at least once.

In particular embodiments, in the method of making the hybrid polymer, steps (b) through (d) are repeated until each amino acid in the donor peptide has been transferred to the hybrid polymer.

In particular embodiments, in the method of making the hybrid polymer, the polymeric seed strand (PSS) includes a natural or synthetic biopolymer.

In particular embodiments, in the method of making the hybrid polymer, the natural biopolymer includes a polynucleotide.

In particular embodiments, in the method of making the hybrid polymer, the polynucleotide includes a single stranded DNA (ssDNA) molecule. Also contemplated are double stranded DNA (dsDNA), and DNA molecules that are partially double-stranded and partially single-stranded.

In particular embodiments, in the method of making the hybrid polymer, the docking linker (DL) includes a multifunctional DL, for instance a bifunctional or trifunctional DL.

In particular embodiments, in the method of making the hybrid polymer, the bifunctional DL includes dibenzocyclooctyne-hexyl-N-succinimidyl (NHS) ester (DBCO-C6-NHS) having the structure:

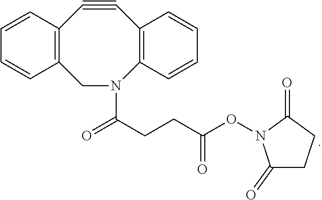 [SMILES: O=C(CCC(=O)N1Cc2ccccc2C#Cc2ccccc21)ON1C(=O)CCC1=O]

In particular embodiments, in the method of making the hybrid polymer, the trifunctional DL is selected from the group consisting of: (a) a DL including amine, methyltetrazine, and dibenzocyclooctyne (DBCO) groups; (b) a DL including amine, DBCO, and tetra C₁-C₄ alkoxysilane groups wherein the tetra C₁-C₄ alkoxysilane group is conjugated to a solid or semi-solid support.

In particular embodiments, in the method of making the hybrid polymer, the DL including amine, methyltetrazine, and DBCO groups has the structure:

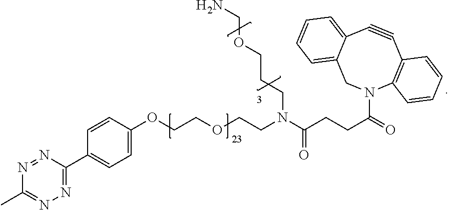

In particular embodiments, in the method of making the hybrid polymer, the DL including amine, DBCO, and tetra C₁-C₄ alkoxysilane groups wherein the tetra C₁-C₄ alkoxysilane group is

In particular embodiments, in the method of making the hybrid polymer, the DL including conjugated to a solid or semi-solid support, has the structure:

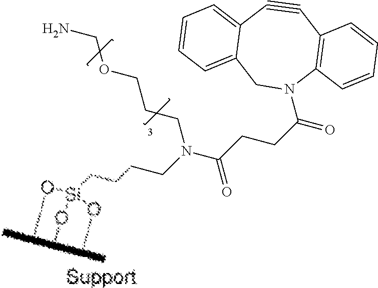

or by the structure:

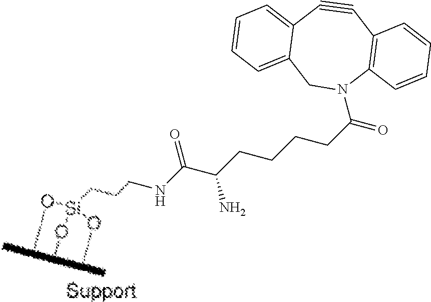 [SMILES: CNC(=O)[C@@H](N)CCCCC(=O)N1Cc2ccccc2C#Cc2ccccc21]

wherein the symbol “ [symbol] ” indicates a solid or semisolid support.

In particular embodiments, in the method of making the hybrid polymer, the DL including two or more functional groups from the following: amines, hydrazine, azides, N-hydroxysuccinimide (NHS), dibenzocyclooctyne (DBCO), alkynes, tetrazine, maleimide, aldehyde, acrylate, TCO, thiol, and alkenes. Photo-reactive crosslinkers, such as photo-reactive azides (phenyl azide, ortho-hydroxyphenyl azide, meta-hydroxyphenyl azide, tetrafluorophenyl azide, ortho-nitrophenyl azide, meta-nitrophenyl azide, and azido-methylcoumarin), diazirine, and psoralen.

In particular embodiments, in the method of making the hybrid polymer, the assembly block (AB) includes a polynucleotide having an isothiocyanate (—N═C═S) (ITC) or isoselenocyanate (—N—C═Se) (ISC) reactive functionality for reacting with the amino group of NTAA of the polypeptide.

In particular embodiments, in the method of making the hybrid polymer, ligating the AB to the proximal end of the PSS includes an enzymatic or a non-enzymatic chemical ligation.

In particular embodiments, in the method of making the hybrid polymer, the enzymatic ligation includes, stick end ligation, blunt end ligation, splint ligation, or single-strand ligation.

In particular embodiments, in the method of making the hybrid polymer, the non-enzymatic ligation includes click chemistry.

particular embodiments, in the method of making the hybrid polymer, cleaving the bond between the NTAA that is attached to the AB and the prior amino acid attached to the NTAA to expose a new NTAA includes Edman degradation, Edman degradation enzyme reaction, or a similar process.

The current disclosure also provides a hybrid polymer made by any of the aforementioned processes.

The current disclosure also provides a method of sequencing a donor polypeptide, including: preparing a hybrid polymer according to any of the aforementioned methods; and passing the hybrid polymer through a nanopore to read a single amino acid at a time, thereby sequencing the polypeptide.

In particular embodiments, in the method of sequencing a donor polypeptide, passing the polymer through the nanopore simplifies an occupancy issue inherent in nanopore peptide analysis by eliminating (minimizing, or rendering subtractable) electrical signal contribution of adjacent amino acids, and allowing nanopore-based peptide sequencing at single-amino acid resolution.

Thus, provided herein are hybrid polymers including at least one repeat unit having the structure of any one of Formulae (I)-(X), wherein the symbol “ [symbol] ” represents a single-stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein; [symbol] indicates that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid.

In examples of hybrid polymer, the protection or the modification of the side chain is to minimize or prevent unwanted side reactions or to render the side chain more detectable than the unprotected or unmodified side chain.

In examples of hybrid polymer, the natural or synthetic biopolymer includes repeat units of nucleic acids and/or amino acids.

In examples of hybrid polymer, the natural biopolymer includes a polynucleotide.

In examples of hybrid polymer, the polynucleotide includes a single-stranded DNA or RNA.

In examples of hybrid polymer, the repeat units include at least two selected from the group of Formula (I), Formula (II), Formula (III), Formula (IV), Formula (IX), and Formula (X).

In examples of hybrid polymer, the repeat units include at least two selected from the group of Formula (I), Formula (III), and Formula (IX).

Also provided herein are polymers including at least one repeat unit having the structure of any one of Formulae (XI)-(XX), wherein the symbol “ [symbol] ” represents a double stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein. L is the structure resulting from a reactive functionality on the AB or hybrid polymer and a reactive functionality on the NTAA of the peptide or resultant SCD. The structure includes any structures or spacers included in the creation of the reactive functionalities; [symbol] indicates that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid.

In examples of hybrid polymer, the protection or the modification of the side chain is to minimize or prevent unwanted side reactions or to render the side chain more detectable than the unprotected or unmodified side chain.

In examples of hybrid polymer, the natural of synthetic biopolymer includes repeat units of nucleic acids and/or amino acids.

In examples of hybrid polymer, the natural biopolymer includes a polynucleotide.

In examples of hybrid polymer, the polynucleotide includes a double-stranded DNA or RNA.

In examples of hybrid polymer, the repeat units include at least two selected from the group of Formula (XI), Formula (XII), Formula (XIII), Formula (XVI), Formula (XIX), and Formula (XX).

In examples of hybrid polymer, the repeat units include at least two selected from the group of Formula (XI), Formula (XII) and Formula (XIX).

Also provided herein are method of making a hybrid polymer, which methods include: modifying a donor polypeptide having a C-terminal amino acid (CTAA) and an N-terminal amino acid (NTAA), such that in the modification, the adjacent amino acids of the donor peptide are separated by segments of a polymeric seed strand (PSS) having a distal end and a proximal end, and wherein in the hybrid polymer the identify and relative position of each amino acid of the donor polypeptide is retained.

By way of example of such method embodiments, the modifying may include: (a) attaching the distal end of the PSS to a docking linker (DL) that connects the CTAA of the donor polypeptide and the PSS, and wherein the other end of the polymeric seed strand is the proximal end; (b) attaching an assembly block (AB) to the NTAA of the donor polypeptide by reaction between a reactive functionality in the NTAA and a reactive functionality in the AB; (c) ligating the AB to the proximal end of the polymeric seed strand; (d) cleaving the bond between the NTAA that is attached to the AB and the prior amino acid attached to the NTAA to expose a new NTAA; and (e) repeating steps (b) through (d) at least once. Optionally, such methods may include repeating steps (b) through (d) until a plurality of amino acids in the donor peptide have been transferred to the hybrid polymer.

In example method embodiments, the polymeric seed strand (PSS) includes a natural or synthetic biopolymer.

In example method embodiments, the natural biopolymer includes a polynucleotide.

In example method embodiments, the polynucleotide includes a single stranded DNA (ssDNA) molecule.

In example method embodiments, the docking linker (DL) includes a multifunctional DL, such as a bifunctional DL or a trifunctional DL.

In example method embodiments, the bifunctional DL includes dibenzocyclooctyne-hexyl-N-succinimidyl (NHS) ester (DBCO-C6-NHS) having the structure:

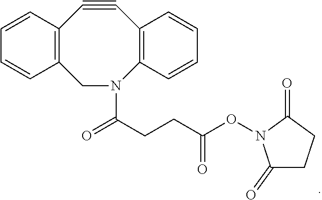 [SMILES: O=C(CCC(=O)N1Cc2ccccc2C#Cc2ccccc21)ON1C(=O)CCC1=O]

In example method embodiments, the trifunctional DL includes amino, DBCO, and tetra C₁-C₄ alkoxysilane groups wherein the tetra C₁-C₄ alkoxysilane group is conjugated to a solid or semisolid support.

In example method embodiments, the trifunctional DL has the structure:

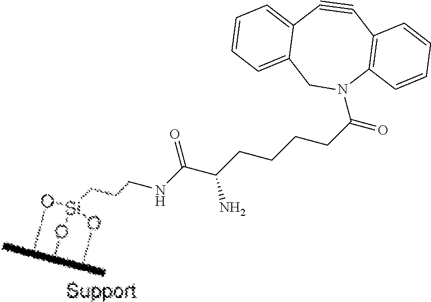 [SMILES: CNC(=O)[C@@H](N)CCCCC(=O)N1Cc2ccccc2C#Cc2ccccc21]

wherein the symbol “ [symbol] ” indicates a solid or semisolid support.

In example method embodiments, the assembly block (AB) includes a polynucleotide having an isothiocyanate (—N═C═S) (ITC) or isoselenocyanate (—N—C═Se) (ISC) reactive functionality for reacting with the amino group of NTAA of the polypeptide.

In example method embodiments, ligating the AB to the proximal end of the PSS includes an enzymatic or a non-enzymatic chemical ligation.

In example method embodiments, enzymatic ligation includes, stick end ligation, blunt end ligation, splint ligation, or single-strand ligation.

In example method embodiments, the non-enzymatic chemical ligation includes click chemistry.

In example method embodiments, cleaving the bond between the NTAA that is attached to the AB and the prior amino acid attached to the NTAA to expose a new NTAA takes includes Edman degradation, Edman degradation enzyme reaction, or a similar process.

Also provided are hybrid polymers made by any of the described methods.

Yet another embodiment is a method of sequencing a donor polypeptide, including: preparing a hybrid polymer according to any of the described method embodiments; and analyzing the hybrid polymer, wherein the analyzing includes identifying two or more amino acids of the donor polypeptide in order along the hybrid polymer. In examples of such method embodiments, analyzing the hybrid polymer includes passing the hybrid polymer through a nanopore to sequentially identify single amino acids of the donor polypeptide, thereby sequencing the polypeptide. In examples of such method embodiments, spacing of the amino acids along the hybrid polymer is sufficient to reduce, enhance, or otherwise alter (modify) electrical signal contribution of adjacent amino acids, thereby allowing nanopore-based peptide sequencing at single-amino acid resolution. In examples of such method embodiments, the hybrid polymer is a polynucleotide, and wherein spacing of the amino acids along the hybrid polymer is sufficient to allow a helicase enzyme to slow down the rate and movement step size of the hybrid polymer through the nanopore. By way of example, analyzing the hybrid polymer includes SMRT sequencing.

Additional embodiments are kits for performing a method of any of the described embodiments, which kit includes: one or more polymeric seed strand (PSS), each having a distal end and a proximal end; and one or more assembly blocks, conjugation reagents, ligation reagents, Edman cleavage reagents, wash buffers, solid supports, listings of barcodes, and/or analysis software.

Also provide are methods of sequencing a polypeptide, including: expanding distance between each amino acid of the polypeptide by attaching each amino acid in order to a (non-protein) polymer molecule (which optionally may have a nucleic acid backbone) to produce a hybrid polymer; and analyzing the hybrid polymer, for instance by passing the hybrid polymer through a nanopore to read a single amino acid at a time or by SMRT sequencing. In embodiments of such methods, the polymer molecule has a nucleic acid backbone that is single stranded or double stranded.

In any of the described embodiments, the amino acids of the polypeptide (e.g., the donor peptide) are attached to (transferred) the polymer molecule in order (that is, in the order in which the amino acids occur in the donor peptide). In various embodiments, the amino acids are moved from the peptide to the hybrid polymer in order from the N-terminal end to the C-terminal end of the polypeptide; or in order from the C-terminal end to the N-terminal end of the polypeptide.

Yet another embodiment is a method of sequencing a polypeptide, wherein the polypeptide has a C-terminal amino acid (CTAA) and an N-terminal amino acid (NTAA), the method including: producing a hybrid polymer by: (a) attaching a flexible linker to the CTAA of the polypeptide; (b) attaching an (isothiocyanate (ITC) or an analogue thereof)-DNA conjugate to the NTAA of the polypeptide; (c) aligning the end of the linker proximal to the NTAA and proximal to the end of the (ITC or an analogue thereof)-DNA conjugate though a bridge oligomer; (d) ligating the linker proximal to the NTAA and the end of the (ITC or an analogue thereof)-DNA; (e) cleaving the NTAA; (f) attaching an (ITC or an analogue thereof)-DNA conjugate to the new NTAA of the polypeptide; and (g) repeating steps (c) through (e) until the end of polypeptide is reached and there is no new NTAA left from the original polypeptide for attaching to an (ITC or an analogue thereof)-DNA conjugate; to generate a linearized and expanded polypeptide chain ready for sequencing; and analyzing the hybrid polymer to identify each amino acid of the donor peptide, thereby sequencing the polypeptide.

### BRIEF DESCRIPTION OF THE DRAWINGS

FIG. 1 Workflow of an embodiment of PROtein Sequencing by Expansion (PROSE).

FIG. 2 Examples of different assembly schemes for PROSE construct embodiments. Possible substructures of a PROSE construct include polymeric and monomeric sequences that may or may not be designed to carry amino acid side chain decorations (SCDs), which are the amino acid moiety cleaved from the appropriate terminus (C- or N-, depending on the conjugation and degradation scheme that is used for a given experiment) of a donor peptide or protein (DP).

FIG. 3 Enzymatic and non-enzymatic ligations schemes for monomeric or polymeric assembly blocks (ABs) conjugated to a donor peptide (DP) and a solid or semi-solid substrate. These schemes also apply to embodiments where no solid or semi-solid substrate is used and, instead, the AB-DP conjugates are freely dispersed in solution.

FIG. 4 Abbreviations for modified bases, linkers, spacers, and terminal modifications included within oligonucleotide sequences in addition to standard letter abbreviations.

FIG. 5 Oligonucleotide complementarity of the example provided in Table 1.

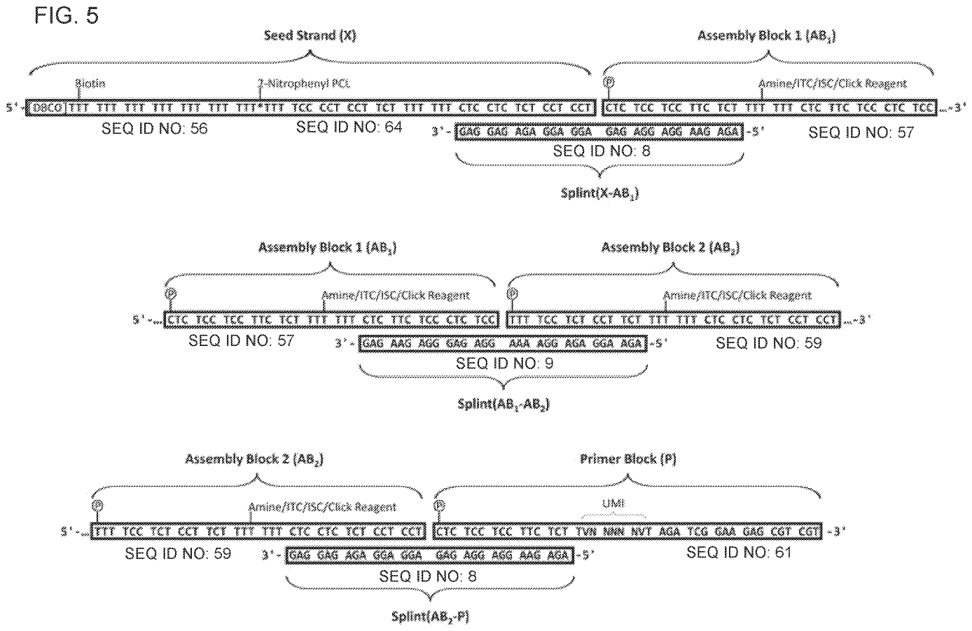

FIGS. 6A-6B Reactive functional group modified nucleobases (FIG. 6A) and backbones (FIG. 6B).

FIG. 7 Conjugation of the assembly block-isothiocyanate (AB-ITC) with the N-terminal primary amine of a donor peptide or protein (DP) yields an AB-thiourea-DP (AB-TU-DP) conjugate. Upon cleavage of the N-terminal amino acid (NTAA), an AB-thiazolinone (AB-TZ) structure forms (which under certain conditions can be unstable), which is hydrolyzed to form an AB-thiocarbamate (AB-TH). Under heating and/or acidic conditions the AB-TC can be converted into the more thermodynamically stable AB-thiohydantoin (AB-TH) form. TZ, TC, and TH are all different forms of amino acid side chain decorations (SCDs).

FIG. 8 Schematic structures of representative multifunctional docking linkers (DLs). Example connections to a tri-functional DL (top) and to a bifunctional DL (bottom) are illustrated.

FIG. 9 Examples of multifunctional docking linkers (DLs). A tri-functional DL containing amine, methyltetrazine, and dibenzocyclooctyne (DBCO) groups (top). A tri-functional DL containing amine, DBCO, and tetraalkoxysilane groups wherein the tetraalkoxysilane group is conjugated to a solid or semi-solid support (middle). An alternate tri-functional DL containing amine, DBCO, and tetraalkoxysilane groups wherein the tetraalkoxysilane group is conjugated to a solid or semi-solid support (bottom). The tetraalkoxysilane moieties of these DLs are designed to be either tetramethoxysilane or tetraethoxysilane.

FIG. 10 Stability of three oligonucleotide 96-mers differing by purine content, low purine (LP; SEQ ID NO: 1), high purine (HP; SEQ ID NO: 2), and no purine (NP; SEQ ID NO: 3), incubated in 1% v/v aqueous trifluoroacetic acid (TFA) for 24 h at 50° C. or 95° C. The controls were incubated at room temperature for 24 h without TFA. Complete strand scission of LP (SEQ ID NO: 1) and HP (SEQ ID NO: 2) was observed at both 50° C. and 95° C. Complete strand scission of NP (SEQ ID NO: 3) was observed at 95° C., but little to no strand scission was observed for NP in 1% TFA at 50° C.

FIG. 11 An example of the PROSE workflow was performed with samples of partially-assembled PROSE constructs taken at four different intervals (left gel panel): (AB₁) supernatant of the Edman degradation reaction (E.D. Sup.) after ligation of the first assembly block (AB) in the workflow; (AB₂) supernatant of the Edman degradation reaction after ligation of the second conjugated oligo, AB₂, in the workflow; and (X-AB₁) substrate cleavage after the first assembly cycle to form X-AB₁, (X-AB₁-AB₂) substrate cleavage after the second assembly cycle to form X-AB₁-AB₂. These fractions were run on a 15% tris-borate-urea gel to validate proximity ligation of the AB₁-peptide (AB₁-TU-DP) conjugates. Both ligated products (extensions) and impurities were retained in the X-AB₁ and X-AB₁-AB₂ samples, and the final construct yielded sequencing results, as depicted in FIG. 18 and FIG. 19. To confirm peptide conjugation occurred in the absence of AB-AB dimers, a mock reaction was performed in suspension. The mock substrate-bound seed strand (X=PSS (polymeric seed strand)) depicts a different length profile than AB₁-ITC (B). When AB₁-ITC is exposed to peptides in solution at pH 8.5 (0.1% v/v triethylamine in water), an oligo-peptide conjugate band is present above the 40 base ladder mark (AB₁-Peptide). To control for potential dimer formation between ABs, the same reaction was performed without peptides. Some dimers may have formed but are obfuscated by impurities in the oligonucleotide solution (AB₁-dimer).

FIG. 12 Gel electrophoresis of assembly block-thiourea-donor peptide (AB-TU-DP) conjugates under various Edman degradation conditions. AB-peptide conjugates (Mock) were created as previously described (0.1% v/v triethylamine in water). Control band (Cntl) portrays the AB-ITC without peptide present.

FIG. 13 Densitometry of relative depletion of assembly block (AB)-TU-peptide conjugates after Edman degradation, which is indicative of the successful cleavage of the N-terminal amino acid (NTAA).

FIG. 14 Various examples of conjugation of an assembly block-isothiocyanate (AB-ITC) conjugation to a peptide and examples of Edman degradation conditions.

FIG. 15 Additional examples of Edman degradation conditions.

FIG. 16 Densitometry of peptide oligo-conjugate depletion after alternative Edman degradation strategies continued.

FIG. 17 Averaged raw nanopore signal showing differential signal response according to the presence or absence of a SCD on the PROSE construct. The signal is acquired by the sample preparation described in Example 9.

FIG. 18 Number of nanopore reads of PROSE constructs assembled with (solid bars) or without (hollow bars) donor peptide. The signal is acquired by the sample preparation described in Example 8.

FIG. 19 Sequence alignment of reads of the PROSE construct. Gaps in the alignment correspond to the presence of an amino acid side chain decoration (SCD) borne by the PROSE construct. SEQ ID NOs: 31-55 are illustrated (in order).

FIG. 20 Processed nanopore current signal of the PROSE construct. Signal variations caused by canonical thymidine bases (TTTTTTT) and a non-canonical base inserted in a string of Ts (TTT-X-TTT) are also shown. Non-canonical bases include T_{C6N}, and its amino acid side chain decorations (SCD), including Glycine, Histidine and Arginine.

FIG. 21 Nanopore current signal for canonical and non-canonical bases are extracted and transformed using principal component analysis (PCA). The reads cluster well and show good separation between the canonical and the various non canonical modifications of the DNA. “Z” can be a hydrogen atom, single amino acid, peptide of about 2 to about 200 amino acids, or a protein of up to about 2000 amino acids. The amino acid can be natural, unnatural, or synthetic.

FIG. 22 Example reaction scheme for C-terminal degradation to form an assembly block-alkylated thiohydantoin (AB-ATH), where ATH is a type of side chain decoration (SCD), and the donor peptide-thiohydantoin (DP-TH) conjugate. The DP-TH is immediately ready for the next alkylation reaction with AB-Acyl Halide or AB-Tosylate (AB-OTs), without the need for re-activation with acetic anhydride and triphenylgermanyl isothiocyanate (Ph₃Ge-ITC).

FIG. 23 illustrates a reaction scheme to convert the amino group of the AB1 (SEQ ID NO: 5, P003 in Table 1) to isothiocyanate (ITC).

FIG. 24 illustrates an example scheme for producing a multifunctional linker via solid phase oligonucleotide synthesis (SPOS).

FIG. 25 illustrates an example scheme for producing a multifunctional docking linker with a photocleavable moiety.

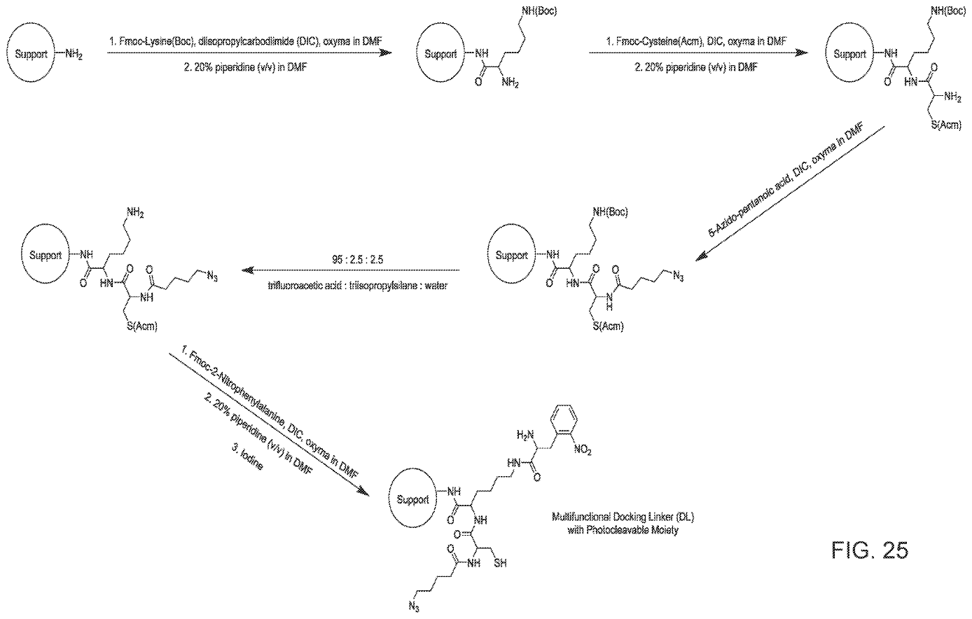

### REFERENCES TO SEQUENCES

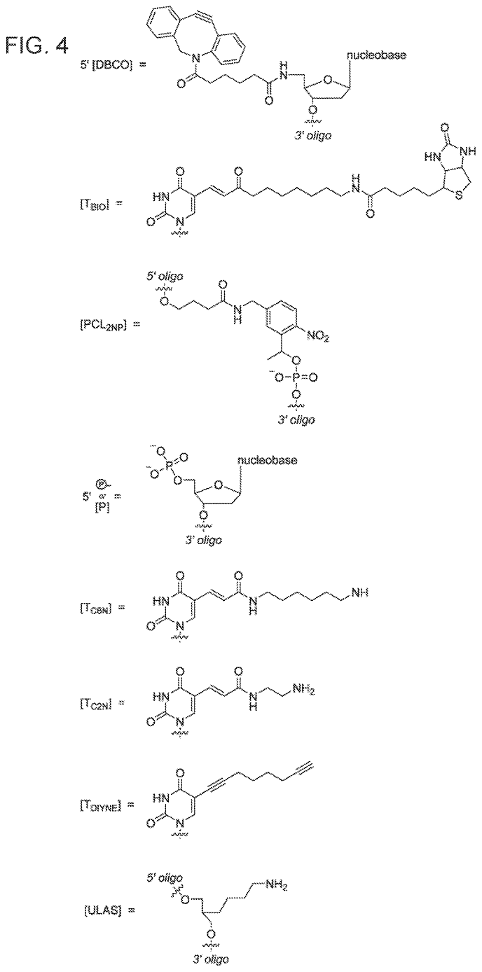

The nucleic acid and/or amino acid sequences described herein are shown using standard letter abbreviations, as defined in 37 CFR § 1.822. Only one strand of each nucleic acid sequence is shown, but the complementary strand is understood as included in embodiments where it would be appropriate. In the sequences: [P]=5′ phosphorylation, added so the oligo can be a substrate for T4 DNA ligase. [T_{BIO}]=Int Amino Modifier C6 dT; (idtdna.com/site/Catalog/Modifications/Product/1388). [Tc_{2N}]=Amino-Modifier C2 dT (glenresearch.com/amino-modifiers/10-1037.html). [ULAS]=Int Uni-Link™ Amino Modifier (idtdna.com/site/Catalog/Modifications/Product/1440). [T_{DIYNE}]=Int 5—Octadiynyl dU (idtdna.com/site/Catalog/Modifications/Product/3508). [DBCO]=5′ dibenzocyclooctyne (idtdna.com/pages/education/decoded/article/need-α-non-standard-modification). [PCL_{2NP}]=internal 2-nitrobenzyl photocleavable linker (idtdna.com/pages/education/decoded/article/modification-highlight-photo-cleavable-spacer). [T_{C6N}]=internal hexyl-amine modified thymine (idtdna.com/site/catalog/modifications/product/1388). [N3]=azido group (idtdna.com/site/catalog/modifications/product/3770). [5PCBio]=5′ photocleavable biotin (idtdna.com/Site/Catalog/Modifications/Product/2291). [iSpC3]=internal C3 phosphoramidite spacer (idtdna.com/site/Catalog/Modifications/Product/1056). In addition, abbreviations for modified bases, linkers, spacers, and terminal modifications are provided in FIG. 4.

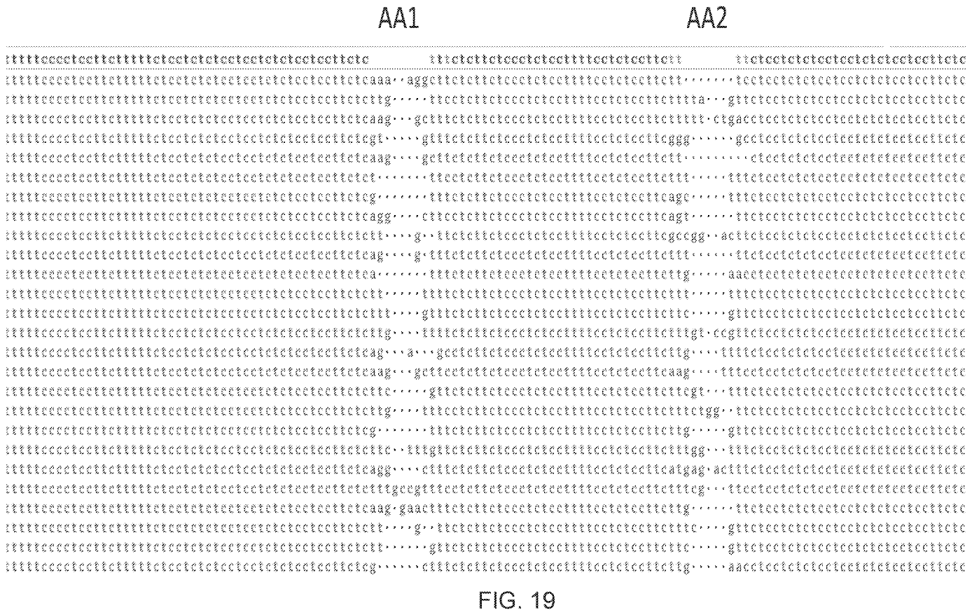

SEQ ID NO: 1 is the nucleotide sequence of low purine content primer LP:
AGCTTTTTGTTTTTGTTGTTTTTTTTGTTTTTTTTGTTTTTGTTTTTGTTTTTGTTTTTGTT
TTTGTTTTTGTGTTTTTGTTTTTGTTTTTGTCTA
SEQ ID NO: 2 is the nucleotide sequence of high purine content primer HP:
AGACAAAAACAAAAACAAAAACACAAAAACAAAAACAAAAACAAAAACAAAAACAAAAACAA
AAAAAACAAAAAAAACAACAAAAACAAAAAGCTA
SEQ ID NO: 3 is the nucleotide sequence of no purine content primer NP:
TCCTTTTTCTTTTTCTTCTTTTTTTTCTTTTTTTTCTTTTTCTTTTTCTTTTTCTTTTTCTTTTTCT
TTTTCTCTTTTTCTTTTTCTTTTTCTCTT
SEQ ID NO: 4 (first part, before linker) and SEQ ID NO: 62 (second part; after linker)
together form the nucleotide sequence of PSS as the X strand. P001:
[DBCO]T[T_{BIO}]TTTTTTTTTTTTTTTTTTT[PCL_{2NP}]
TTTTCCCCTCCTTCTTTTTTTCTCCTCTCTCCTCCT3′
SEQ ID NO: 5 is the nucleotide sequence of AB with a C6 amine. P003:
[P]CTCTCCTCCTTCTCTTT[T_{C6N}]TTTCTCTTCTCCCTCTCC3′
SEQ ID NO: 6 is the nucleotide sequence of AB with a C6 amine. P005:
[P]TTTTCCTCTCCTTCTTT[T_{C6N}]TTTCTCCTCTCTCCTCCT 3′
SEQ ID NO: 7 is the nucleotide sequence of Primer with random UMI sequence. P007:
[P]CTCTCCTCCTTCTCTTVYYYYYVTAGATCGGAAGAGCGTCGT 3′ (where Y can be T or C
and V can be A, C, or G).
SEQ ID NO: 8 is the nucleotide sequence of Nucleotide for splint ligation. P002:
′5 AGAGAAGGAGGAGAGAGGAGGAGAGAGGAG
SEQ ID NO: 9 is the nucleotide sequence of Nucleotide for splint ligation. P004:
′5 AGAAGGAGAGGAAAAGGAGAGGGAGAAGAG
SEQ ID NO: 10 is the nucleotide sequence of AGA P014:
[P]CTCTCCTCCTTCTCTTT[T_{C2N}]TTTCTCTTCTCCCTCTCC 3′
SEQ ID NO: 11 is the nucleotide sequence of P015:
[P] TTTTCCTCTCCTTCTTT[T_{C2N}]TTTCTCCTCTCTCCTCCT 3′
SEQ ID NO: 12 (first part, before linker) and SEQ ID NO: 63 (second part, after linker)
together form the nucleotide sequence of AB with a UniLink amine linker.
[P]CTCTCCTCCTTCTCTTT/[ULAS]TTTCTCTTCTCCCTCTCC 3′
SEQ ID NO: 13 is the nucleotide sequence of AB with an alkyne group.
[P]CTCTCCTCCTTCTCTTT/[T_{DIYNE}]TTTCTCTTCTCCCTCTCC 3′
SEQ ID NO: 14 is the nucleotide sequence of Nucleotide for splint ligation; P030:
′5 GGAGAGAGGAGGAGAGAGGAGAAA
SEQ ID NO: 15 is the nucleotide sequence of Nucleotide for splint ligation; P031:
′5 AGGAAAAGGAGAGGGAGAAGAGA
SEQ ID NO: 16 is the nucleotide sequence of Primer with specific UMI sequence P020:
[P]CTC TCC TCC TTC TCT TATGCGATCGGAAT AGA TCG GAA GAG CGT CGT 3′
SEQ ID NO: 17 is the nucleotide sequence of Primer with specific UMI sequence P021:
[P]CTC TCC TCC TTC TCT TCCGGATTAGACGT AGA TCG GAA GAG CGT CGT 3′
SEQ ID NO: 18 is the nucleotide sequence of Primer with specific UMI sequence. P022:
[P]CTC TCC TCC TTC TCT TCGTCATAGTGGAT AGA TCG GAA GAG CGT CGT 3′
SEQ ID NO: 19 is the nucleotide sequence of Primer with specific UMI sequence. P023:
[P]CTC TCC TCC TTC TCT TGAGATAGGATGTT AGA TCG GAA GAG CGT CGT 3′
SEQ ID NO: 20 is the nucleotide sequence of Primer with specific UMI sequence. P024:
[P]CTC TCC TCC TTC TCT TGGCAGCAATCCAT AGA TCG GAA GAG CGT CGT 3′
SEQ ID NO: 21 is the nucleotide sequence of Primer with specific UMI sequence. P025:
[P]CTC TCC TCC TTC TCT TGCTATAATAGTGT AGA TCG GAA GAG CGT CGT 3′
SEQ ID NO: 22 is the nucleotide sequence of Primer with specific UMI sequence. P026:
[P]CTC TCC TCC TTC TCT TATCAGTCAGAGTT AGA TCG GAA GAG CGT CGT 3′
SEQ ID NO: 23 is the nucleotide sequence of Primer with specific UMI sequence. P027:
[P]CTC TCC TCC TTC TCT TACCGTTACGCCAT AGA TCG GAA GAG CGT CGT 3′
SEQ ID NO: 24 is the nucleotide sequence of Primer with specific UMI sequence. P028:
[P]CTC TCC TCC TTC TCT TCCGAACGGCGCGT AGA TCG GAA GAG CGT CGT 3′
SEQ ID NO: 25 is the nucleotide sequence of Primer with specific UMI sequence. P029:
[P]CTC TCC TCC TTC TCT TATCATCAGAGGAT AGA TCG GAA GAG CGT CGT 3′
SEQ ID NO: 26 is a representative peptide with an azido group at the C-terminal Lysine:
TrpGlySerGlyGlySerGlyGlySerAspAspLys[N3]
SEQ ID NO: 27 is the amino acid sequence: TrpGlyGlySerGlyGlySerAspCys
SEQ ID NO: 28 is the amino acid sequence: GlyGlyGlySerGlyGlySerAspCys
SEQ ID NO: 29 is the amino acid sequence: ArgGlyGlySerGlyGlySerAspCys
SEQ ID NO: 30 is the amino acid sequence: HisGlyGlySerGlyGlySerAspCys
SEQ ID NOs: 31-55 are sequencing reads illustrated in FIG. 19:
SEQ ID NO: 31:
TTTTTCCCCTCCTTCTTTTTCTCTCCTCTCTCCTCCTCTCTCCTCCTTCTCAAAAGGCTTCTC
TTCTCCCTCTCCTTTTCCTCTCCTTCTTTCCTCCTCTCTCCTCCTCT
SEQ ID NO: 32:
TTTTTCCCCTCCTTCTTTTTCTCCTCTCTCCTCCTCTCTCCTCCTTCTCTTGTTCCTCTTCTC
CCTCTCCTTTTCCTCTCCTTCTTTTAGTTCTCCTCTCTCCTCCTCTC
SEQ ID NO: 33:
TTTTTCCCCTCCTTCTTTTTCTCCTCTCTCCTCCTCTCTCCTCCTTCTCAAGGCTTTCTCTTC
TCCCTCTCCTTTTCCTCTCCTTCTTTTTCTGACCTCCTCTCTCCTCC
SEQ ID NO: 34:
TTTTTCCCCTCCTTCTTTTTCTCCTCTCTCCTCCTCTCTCCTCCTTCTCGTGTTTCTCTTCTC
CCTCTCCTTTTCCTCTCCTTCGGGGCCTCCTCTCTCCTCCTCTCTCC
SEQ ID NO: 35:
TTTTTCCCCTCCTTCTTTTTTTCTCCTCTCTCCTCCTCTCTCCTCCTTCTCAAGGCTTCTCTT
CTCCCTCTCCTTTTCCTCTCCTTCTTCTCCTCTCTCCTCCTCTCTCC
SEQ ID NO: 36:
TTTTTCCCCTCCTTCTTTTTCTCCTCTCTCCTCCTCTCTCCTCCTTCTCTTTCCTCTTCTCCCT
CTCCTTTTCCTCTCCTTCTTTTTTCTCCTCTCTCCTCCTCTCTCCT
SEQ ID NO: 37:
TTTTTCCCCTCCTTCTTTTTTCTCCTCTCTCCTCCTCTCTCCTCCTTCTCGTTTCTCTTCTCC
CTCTCCTTTTCCTCTCCTTCAGCTTTCTCCTCTCTCCTCCTCTCTCC
SEQ ID NO: 38:
TTTTTCCCCTCCTTCTTTTTTCTCCTCTCTCCTCCTCTCTCCTCCTTCTCAGGCTTCCTCTTC
TCCCTCTCCTTTTCCTCTCCTTCAGTTTCTCCTCTCTCCTCCTCTCT
SEQ ID NO: 39:
TTTTTCCCCTCCTTCTTTTTTCTCCTCTCTCCTCCTCTCTCCTCCTTCTCTTGTTCTCTTCTCC
CTCTCCTTTTCCTCTCCTTCGCCGGACTTCTCCTCTCTCCTCCTCT
SEQ ID NO: 40:
TTTTTCCCCTCCTTCTTTTTTTTCTCCTCTCTCCTCCTCTCTCCTCCTTCTCAGGTTTCTCTTC
TCCCTCTCCTTTTCCTCTCCTTCTTTTTCTCCTCTCTCCTCCTCTC
SEQ ID NO: 41:
TTTTTCCCCTCCTTCTTTTTTCTCCTCTCTCCTCCTCTCTCCTCCTTCTCATTTCTCTTCTCCC
TCTCCTTTTCCTCTCCTTCTTGAACCTCCTCTCTCCTCCTCTCTCC
SEQ ID NO: 42:
TTTTTCCCCTCCTTCTTTTTTCTCCTCTCTCCTCCTCTCTCCTCCTTCTCTTTTTTCTCTTCTC
CCTCTCCTTTTCCTCTCCTTCTTTTTTCTCCTCTCTCCTCCTCTCT
SEQ ID NO: 43:
TTTTTCCCCTCCTTCTTTTTTTCTCCTCTCTCCTCCTCTCTCCTCCTTCTCTTTGTTTCTCTTC
TCCCTCTCCTTTTCCTCTCCTTCTTCGTTCTCCTCTCTCCTCCTCT
SEQ ID NO: 44:
TTTTTCCCCTCCTTCTTTTTTCTCCTCTCTCCTCCTCTCTCCTCCTTCTCTTGTTTTCTCTTCT
CCCTCTCCTTTTCCTCTCCTTCTTTGTCCGTTCTCCTCTCTCCTCC
SEQ ID NO: 45:
TTTTTCCCCTCCTTCTTTTTCTCCTCTCTCCTCCTCTCTCCTCCTTCTCAGAGCCTCTTCTCC
CTCTCCTTTTCCTCTCCTTCTTGTTTTCTCCTCTCTCCTCCTCTCTC
SEQ ID NO: 46:
TTTTTCCCCTCCTTCTTTTTTCTCCTCTCTCCTCCTCTCTCCTCCTTCTCAAGGCTTCCTCTT
CTCCCTCTCCTTTTCCTCTCCTTCAAGTTTCCTCCTCTCTCCTCCTC
SEQ ID NO: 47:
TTTTTCCCCTCCTTCTTTTTTTCTCCTCTCTCCTCCTCTCTCCTCCTTCTCTTCGTTCTCTTCT
CCCTCTCCTTTTCCTCTCCTTCTTCGTTTTCTCCTCTCTCCTCCTC
SEQ ID NO: 48:
TTTTTCCCCTCCTTCTTTTTTCTCCTCTCTCCTCCTCTCTCCTCCTTCTCTTGTTTCCTCTTCT
CCCTCTCCTTTTCCTCTCCTTCTTTCTGGTTCTCCTCTCTCCTCCT
SEQ ID NO: 49:
TTTTTCCCCTCCTTCTTTTTTTCTCCTCTCTCCTCCTCTCTCCTCCTTCTCGTTTCTCTTCTCC
CTCTCCTTTTCCTCTCCTTCTTGGTTCTCCTCTCTCCTCCTCTCTC
SEQ ID NO: 50:
TTTTTCCCCTCCTTCTTTTTTCTCCTCTCTCCTCCTCTCTCCTCCTTCTCTTCTTTGTTCTCTT
CTCCCTCTCCTTTTCCTCTCCTTCTTTGGTTTCTCCTCTCTCCTCC
SEQ ID NO: 51:
TTTTTCCCCTCCTTCTTTTTCTCCTCTCTCCTCCTCTCTCCTCCTTCTCAGGCTTTCTCTTCT
CCCTCTCCTTTTCCTCTCCTTCATGAGACTTTCTCCTCTCTCCTCCT
SEQ ID NO: 52:
TTTTTCCCCTCCTTCTTTTTCTCCTCTCTCCTCCTCTCTCCTCCTTCTCTTTGCCGTTTCCTC
TTCTCCCTCTCCTTTTCCTCTCCTTCTTTCGTTCCTCCTCTCTCCTC
SEQ ID NO: 53:
TTTTTCCCCTCCTTCTTTTTTTCTCCTCTCTCCTCCTCTCTCCTCCTTCTCAAGGAACTTTCT
CTTCTCCCTCTCCTTTTCCTCTCCTTCTTGTTCTCCTCTCTCCTCCT
SEQ ID NO: 54:
TTTTTCCCCTCCTTCTTTTTTTCTCCTCTCTCCTCCTCTCTCCTCCTTCTCTTGTTCTCTTCTC
CCTCTCCTTTTCCTCTCCTTCTTTCGTTCTCCTCTCTCCTCCTCTC
SEQ ID NO: 55:
TTTTTCCCCTCCTTCTTTTTCTCCTCTCTCCTCCTCTCTCCTCCTTCTCTTCCTCCTTCTCTTT
TTTCCTCTTCTCCCTCTCCTTTTCCTCTCCTTCTTTTTTCTCCTCT
SEQ ID NO: 56 (first part, before linker) and SEQ ID NO: 64 (second part, after linker)
together form the nucleotide sequence:
[DBCO]T[T_{BIO}]TTTTTTTTTTTTTTTTTTT[2-
nitrophenyl_PCL]TTTTCCCCTCCTTCTTTTTTTCTCCTCTCTCCTCCT
SEQ ID NO: 57 (Amine/ITC/ISC/Click Reagent optionally attached at position 18):
[P]CTCTCCTCCTTCTCTTTTTTTCTCTTCTCCCTCTCC
SEQ ID NO: 58 is the amino acid sequence of an exemplary peptide:
ArgGlyGlyGlyArgGlyArgGlyGlyArgGlyGlyGlySerGlyGlyGlySerLys[N3]
SEQ ID NO: 59 (Amine/ITC/ISC/Click Reagent optionally attached at position 18):
[P]TTTTCCTCTCCTTCTTTTTTTCTCCTCTCTCCTCCT
SEQ ID NO: 60 is the amino acid sequence of an exemplary peptide:
GlyGlyArgGlyArgGlyArgGlyGlyArgGlyGlyGlySerGlyGlyGlySer Lys[N3]
SEQ ID NO: 61 is the nucleic acid sequence of a primer with a generic Unique Molecular
Identifier (UMI) (positions 16-24):
[P]CTCTCCTCCTTCTCTTVNNNNNVTAGATCGGAAGAGCGTCGT
SEQ ID NO: 65 is the amino acid sequence of an exemplary peptide:
TrpGlyArgGlyArgGlyArgGlyGlyArgGlyGlyGlySerGlyGlyGlySerLys[N3]
SEQ ID NO: 66 is the amino acid sequence of an exemplary peptide:
LeuGlyArgGlyArgGlyArgGlyGlyArgGlyGlyGlySerGlyGlyGlySerLys[N3]
SEQ ID NO: 67 is the amino acid sequence of an exemplary peptide:
LeuTrpAlaGluPheGlyArgGlyArgGlyArgGlyGlyArgGlyGlyGlySerGlyGlyGlySerLys[N3]
SEQ ID NO: 68 is the nucleotide sequence of oligonucleotide for splint ligation P115:
AGAGAAGGAGGAGAGAGGAGG
SEQ ID NOs: 69 (first part, before linker) and 70 (second part, after linker) together are
the nucleotide sequence of oligonucleotide for with a biotin group and photocleavable
linker P80:
/5PCBio/TTTTCCTCTCCTTCTTT/iSpC3/TTTCTCCTCTCTCCTCCT
SEQ ID NOs: 71 (first part, before linker) and 72 (second part, after linker) together are
the nucleotide sequence of oligonucleotide with barcode for multiplexing C1
[P]TTTTCCTCTCCTTCTTT/iSpC3/TTTGTGTAGTTCCACAGATCGGAAGAGCGTCGT
SEQ ID NOs: 71 (first part, before linker) and 73 (second part, after linker) together are
the nucleotide sequence of oligonucleotide with barcode for multiplexing C2
[P]TTTTCCTCTCCTTCTTT/iSpC3/TTTAGGCTCATAACTAGATCGGAAGAGCGTCGT
SEQ ID NOs: 71 (first part, before linker) and 74 (second part, after linker) together are
the nucleotide sequence of oligonucleotide with barcode for multiplexing C3
[P]TTTTCCTCTCCTTCTTT/iSpC3/TTTGATATGTCTACGAGATCGGAAGAGCGTCGT
SEQ ID NOs: 71 (first part, before linker) and 75 (second part, after linker) together are
the nucleotide sequence of oligonucleotide with barcode for multiplexing C4
[P]TTTTCCTCTCCTTCTTT/iSpC3/TTTTCCTAACTGAGGAGATCGGAAGAGCGTCGT
SEQ ID NOs: 71 (first part, before linker) and 76 (second part, after linker) together are
the nucleotide sequence of oligonucleotide with barcode for multiplexing C5
[P]TTTTCCTCTCCTTCTTT/iSpC3/TTTATCTATGGCATAAGATCGGAAGAGCGTCGT
SEQ ID NOs: 71 (first part, before linker) and 77 (second part, after linker) together are
the nucleotide sequence of oligonucleotide with barcode for multiplexing C6
[P]TTTTCCTCTCCTTCTTT/iSpC3/TTTATACCTGTTGGCAGATCGGAAGAGCGTCGT
SEQ ID NOs: 71 (first part, before linker) and 78 (second part, after linker) together are
the nucleotide sequence of oligonucleotide with barcode for multiplexing C7
[P]TTTTCCTCTCCTTCTTT/iSpC3/TTTTTAAGTCGCGCCAGATCGGAAGAGCGTCGT
SEQ ID NOs: 71 (first part, before linker) and 79 (second part, after linker) together are
the nucleotide sequence of oligonucleotide with barcode for multiplexing C8
[P]TTTTCCTCTCCTTCTTT/iSpC3/TTTCCAGACTAAGACAGATCGGAAGAGCGTCGT
SEQ ID NOs: 71 (first part, before linker) and 80 (second part, after linker) together are
the nucleotide sequence of oligonucleotide with barcode for multiplexing C9
[P]TTTTCCTCTCCTTCTTT/iSpC3/TTTTAAGGTCGCAATAGATCGGAAGAGCGTCGT
SEQ ID NOs: 71 (first part, before linker) and 81 (second part, after linker) together are
the nucleotide sequence of oligonucleotide with barcode for multiplexing C10
[P]TTTTCCTCTCCTTCTTT/iSpC3/TTTCCGTGGATCTAGAGATCGGAAGAGCGTCGT
SEQ ID NOs: 71 (first part, before linker) and 82 (second part, after linker) together are
the nucleotide sequence of oligonucleotide with barcode for multiplexing C11
[P]TTTTCCTCTCCTTCTTT/iSpC3/TTTACAAGATCAATCAGATCGGAAGAGCGTCGT
SEQ ID NOs: 71 (first part, before linker) and 83 (second part, after linker) together are
the nucleotide sequence of oligonucleotide with barcode for multiplexing C12
[P]TTTTCCTCTCCTTCTTT/iSpC3/TTTCCACGATCACGAAGATCGGAAGAGCGTCGT
SEQ ID NO: 84 is the nucleotide sequence of oligonucleotide for splint ligation P145:
CTCCACGACGCTCTTCCGATCT
SEQ ID NO: 85 is the nucleotide sequence of Nucleotide Gblock 1:
GGAGAGGTCTTCGGAGAGACGGAAGAGGTGTAGAAGCCTAGGAACAGGTTAGTATGAGTA
GCTTAAGAATGTAAATTCTGTGATTATAGTGTAGTAATCTCTAATTAACGGTGACGCAAAAT
CAAGCGCAGTGATTTCAACAGATAATGCTGATGGTTTAGGCGTACAATGCGCTGAAGAATA
ATTAAGAAAATAGCACTCCTCGTCGCCTAGAATTACCTACCGGCGTCCACCATACCTTCGA
TTATCGCGCCCACTCTCCCATTAGTCGGCACAGGTGGATGTGTTGCGATAGCCCGCTAAG
ATATTCTAAGGCGTAACGCAGATGAATATTCTACAGAGTTGCCATAGGCGTTGAACGCTTC
ACGGACGATAGGAATTAGCGTATAGAGCGCGTCATCGAAGAGTTATACACTCGTAGTTAAC
ATCTAGCCCGGCTATGCGATCGGAATCGGGTGAGACCTCTCCTTTCCTTCTCCTTTC
SEQ ID NO: 86 is the nucleotide sequence of Nucleotide Gblock 2:
GGAGAGGTCTTCGGAGAGACGGAAGAGGTGTAGAAGCCTAGGAACAGGTTAGTATGAGTA
GCTTAAGAATGTAAATTCTGTGATTATAGTGTAGTAATCTCTAATTAACGGTGACGCAAAAT
CAAGCGCAGTGATTTCAACAGATAATGCTGATGGTTTAGGCGTACAATGCGCTGAAGAATA
ATTAAGAAAATAGCACTCCTCGTCGCCTAGAATTACCTACCGGCGTCCACCATACCTTCGA
TTATCGCGCCCACTCTCCCATTAGTCGGCACAGGTGGATGTGTTGCGATAGCCCGCTAAG
ATATTCTAAGGCGTAACGCAGATGAATATTCTACAGAGTTGCCATAGGCGTTGAACGCTTC
ACGGACGATAGGAATTAGCGTATAGAGCGCGTCATCGAAGAGTTATACACTCGTAGTTAAC
ATCTAGCCCGGCTCCGGATTAGACGTCGGGTGAGACCTCTCCTTTCCTTCTCCTTTC
SEQ ID NO: 87 is the nucleotide sequence of P079:
GGAGGAGAGAGGAGAAAAAAAGAAGGAGAGGAAAAA

### DETAILED DESCRIPTION

The PROSE construct consists of the amino acid side chains from a donor protein or portion of a protein that have been displayed onto a polymer backbone in the same linear sequence and with a larger non-natural spacing interval. The polymer could be a biopolymer such as nucleic acids or amino acids.

There is no need to develop new amino acid specific binders, which are hard to find and validate. Instead, properties of the amino acid side chains are directly detected as an electrical signal as their structures pass through a nanopore.

We have solved the issue of amino acid signal complexity by physically spacing the side chains within a novel construct. Nanopore signal complexity scales exponentially for intact canonical peptides and linearly for the PROSE construct. Therefore, we have greatly simplified the signal complexity problem.

The PROSE construct is compatible with other approaches including sequencing by fluorescence lifetime perturbation and other next generation sequencing methods, such as single molecule real time (SMRT) sequencing or sequencing by synthesis (SBS).

Embodiments of the hybrid polymer-based (PROSE construct) sequencing methods described herein, including certain embodiments where the polymer is polynucleotide, simplify translocation issues that are inherent in nanopore peptide analysis, for instance by allowing a helicase enzyme to slow down the rate and movement step size of the hybrid polymer in the pore.

Likewise, embodiments of the hybrid polymer (PROSE construct) simplify a tertiary structure issue inherent in nanopore peptide analysis by making the protein or peptide more linear or DNA-like—and by spreading apart the individual amino acids for the sequencing analysis, thereby simplifying deconvolution of the nanopore sequencing data.

In provided embodiments, the amino acid sequence as present on a hybrid polymer (PROSE construct) described herein can be identified using single molecule real time (SMRT) sequencing by synthesis methods where the SCD introduces characteristic changes in polymerase kinetics and fluorescence intensity to identify at least a partial protein sequence. Thus, provided herein are hybrid polymers including at least one repeat unit having the structure of any one of Formulae (I)-(X), wherein the symbol “ [symbol] ” represents a single-stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein; [symbol] indicates that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid. In embodiments, L can be attached to a base, sugar, or spacer. In embodiments, each R can independently be a natural or a synthetic moiety; and specifically, embodiments are contemplated where Rs within one example include both natural and synthetic moieties. Single (one) repeat unit refers to formulae structure which is a single amino acid (aa) unit; such single repeat unity can be useful for instance in training cases or assessing cleavage or successive cycles.

Also provided herein are polymers including at least one repeat unit having the structure of any one of Formulae (XI)-(XX), wherein the symbol “ [symbol] ” represents a double stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein. L is the structure resulting from a reactive functionality on the AB or hybrid polymer and a reactive functionality on the NTAA of the peptide or resultant SCD. The structure includes any structures or spacers included in the creation of the reactive functionalities; [symbol] indicates that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid.

Also provided herein are method of making a hybrid polymer, which methods include: modifying a donor polypeptide having a C-terminal amino acid (CTAA) and an N-terminal amino acid (NTAA), such that in the modification, the adjacent amino acids of the donor peptide are separated by segments of a polymeric seed strand (PSS) having a distal end and a proximal end, and wherein in the hybrid polymer the identify and relative position of each amino acid of the donor polypeptide is retained.

Byway of example of such method embodiments, the modifying may include: (a) attaching the distal end of the PSS to a docking linker (DL) that connects the CTAA of the donor polypeptide and the PSS, and wherein the other end of the polymeric seed strand is the proximal end; (b) attaching an assembly block (AB) to the NTAA of the donor polypeptide by reaction between a reactive functionality in the NTAA and a reactive functionality in the AB; (c) ligating the AB to the proximal end of the polymeric seed strand; (d) cleaving the bond between the NTAA that is attached to the AB and the prior amino acid attached to the NTAA to expose a new NTAA; and (e) repeating steps (b) through (d) at least once. Optionally, such methods may include repeating steps (b) through (d) until a plurality of amino acids in the donor peptide have been transferred to the hybrid polymer.

Also provided are hybrid polymers made by any of the described methods.

Yet another embodiment is a method of sequencing a donor polypeptide, including: preparing a hybrid polymer according to any of the described method embodiments; and analyzing the hybrid polymer, wherein the analyzing includes identifying two or more amino acids of the donor polypeptide in order along the hybrid polymer. In examples of such method embodiments, analyzing the hybrid polymer includes passing the hybrid polymer through a nanopore to sequentially identify single amino acids of the donor polypeptide, thereby sequencing the polypeptide. In examples of such method embodiments, spacing of the amino acids along the hybrid polymer is sufficient to reduce, enhance, or otherwise alter (e.g., modify) electrical signal contribution of adjacent amino acids, thereby allowing nanopore-based peptide sequencing at single-amino acid resolution. It is recognized that nanopore signals for amino acids are dependent on the amino acid as well as the context surrounding the amino acid—linkers, spacers nucleobases, and so forth. With the described methods, as long as the context is constant (or determinable) for all the SCDs, the individual amino acid residues can be resolved.»

In examples of such method embodiments, the hybrid polymer is a polynucleotide, and wherein spacing of the amino acids along the hybrid polymer is sufficient to allow a helicase enzyme to slow down the rate and movement step size of the hybrid polymer through the nanopore. By way of example, analyzing the hybrid polymer includes SMRT sequencing.

Additional embodiments are kits for performing a method of any of the described embodiments, which kit includes: one or more polymeric seed strand (PSS), each having a distal end and a proximal end; and one or more assembly blocks, conjugation reagents, ligation reagents, Edman cleavage reagents, wash buffers, solid supports, listings of barcodes, and/or analysis software.

Also provide are methods of sequencing a polypeptide, including: expanding distance between each amino acid of the polypeptide by attaching each amino acid in order to a (non-protein) polymer molecule (which optionally may have a nucleic acid backbone) to produce a hybrid polymer; and analyzing the hybrid polymer, for instance by passing the hybrid polymer through a nanopore to read a single amino acid at a time or by SMRT sequencing. In embodiments of such methods, the polymer molecule has a nucleic acid backbone that is single stranded or double stranded.

Yet another embodiment is a method of sequencing a polypeptide, wherein the polypeptide has a C-terminal amino acid (CTAA) and an N-terminal amino acid (NTAA), the method including: producing a hybrid polymer by: (a) attaching a flexible linker to the CTAA of the polypeptide; (b) attaching an (isothiocyanate (ITC) or an analogue thereof)-DNA conjugate to the NTAA of the polypeptide; (c) aligning the end of the linker proximal to the NTAA and proximal to the end of the (ITC or an analogue thereof)-DNA conjugate though a bridge oligomer; (d) ligating the linker proximal to the NTAA and the end of the (ITC or an analogue thereof)-DNA; (e) cleaving the NTAA; (f) attaching an (ITC or an analogue thereof)-DNA conjugate to the new NTAA of the polypeptide; and (g) repeating steps (c) through (e) until the end of polypeptide is reached and there is no new NTAA left from the original polypeptide for attaching to an (ITC or an analogue thereof)-DNA conjugate; to generate a linearized and expanded polypeptide chain ready for sequencing; and analyzing the hybrid polymer to identify each amino acid of the donor peptide, thereby sequencing the polypeptide.

Aspects of the current disclosure are now described with additional details and options as follows: (I) Definitions; (II) Overview; (Ill) Enzymatic or Chemical Digestion; (IV) Blocking Chemical Reactivity with Protecting Groups; (V) Oligonucleotide Composition; (VI) Oligonucleotide Length; (VII) Thiocarbamate and Thiohydantoin Formation; (VIII) Ligation Schemes; (IX) Barcoding and Unique Molecular Identifiers (UMIs); (X) Docking Linker (DL) Design; (XI) PROSE Construct Assembly from C-Terminal Degradation Scheme; (XII) Analogs of the Isothiocyanato Group; (XIII) Alternative Methods of Amino Acid Cleavage; (XIV) PROSE Synthetic Long Reads and Linked Reads; (XV) Barcode or UMI encoding; (XVI) Alternative Methods to Read the PROSE Construct; (XVII) Single Molecule Real Time (SMRT) Sequencing Technology; (XVIII) Sequencing by Synthesis; (XIX) Spectroscopic Methods; (XX) Other Applications of the PROSE Construct; (XXI) Error Tracking and Monitoring; (XXII) A Polypeptide Backbone for PROSE Construct; (XXIII) Exemplary Linker Structures; (XXIV) Synthesis of Isothiocyanate-Containing Oligonucleotides; (XXV) Synthesis of Multi-Functional Docking Linkers (DLs); (XXVI) Conjugation of Peptide to the Oligonucleotide Seed Strand (X); (XXVII) Conjugation of Oligonucleotide-Isothiocyanate (AB-ITC) to N-Terminal Amino Acid (NTAA); (XXVIII) Ligation of Assembly Block-Isothiocyanate-Peptide (AB-ITC-Peptide) Conjugate to Oligonucleotide Seed Strand (X) or to the Preceding AB or Intervening Monomeric or Polymeric Sequence; (XXIX) Edman Degradation to Remove N-Terminal Amino Acid (NTAA) and Liberate the Assembly Block-Side Chain Decoration (AB-SCD); (XXX) Cycling of this Process to Produce the Amino Acid-Decorated PROSE Construct; (XXXI) Workup of a Representative PROSE Construct; (XXXII) Preparation for Nanopore Sequencing; (XXXIII) Software, Data Processing, and Data Analysis; (XXXIV) Exemplary Embodiments; (XXXV) Experimental Examples; and (XXXVI) Closing Paragraphs. These headings do not limit the interpretation of the disclosure and are provided for organizational purposes only.

### (1) Select Definitions and Acronyms

To facilitate understanding, a number of terms are defined below. Terms used herein (unless otherwise specified) have meanings as commonly understood by a person of ordinary skill in the areas relevant to the present disclosure. The terminology herein is used to describe specific embodiments, but their usage is not intended to be limiting, except as outlined in the claims.

The term “alkyl” refers to a straight or branched hydrocarbon. For example, an alkyl group can have 1 to 6 carbon atoms (i.e., C₁-C₆ alkyl or C₁₋₆ alkyl), 1 to 4 carbon atoms (i.e., C₁-C₄ alkyl or C₁₋₄ alkyl), or 1 to 3 carbon atoms (i.e., C₁-C₃ alkyl or C₁₋₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

The term “amino acid” in general refers to organic compounds that contain at least one amino group (—NH₂), and one carboxyl group (—COOH), where the carboxylic acids are deprotonated at neutral pH, having the basic formula of NH₂CHRCOOH. An amino acid and thus a peptide has an N (amino)-terminal residue region and a C (carboxy)-terminal residue region.

The term “terminal” (referred to as singular terminus and plural termini) refers to the end position of a peptide or protein. The “N terminus” or N terminal amino acid is the one found at the amino den of the peptide, while the “C terminus” or C terminal amino acid is the one found at the carboxy end. The phrase “N-terminal amino acid” refers to an amino acid that has a free amine group and is only linked to one other amino acid by a peptide amide bond in the polypeptide. Optionally, the “N-terminal amino acid” may be an “N-terminal amino acid derivative”. As used herein, an “N-terminal amino acid derivative” refers to an N-terminal amino acid residue that has been chemically modified, for example by an Edman reagent or other chemical in vitro or inside a cell via a natural post-translational modification (e.g. phosphorylation) mechanism.

Amino acids include the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or lie), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

The terms “amino acid sequence”, “peptide”, “peptide sequence”, “polypeptide”, and “polypeptide sequence” are used interchangeably herein to refer to at least two amino acids or amino acid analogs that are covalently linked by a peptide (amide) bond or an analog of a peptide bond. The term peptide includes oligomers and polymers of amino acids or amino acid analogs.

The term peptide also includes molecules that are commonly referred to as peptides, which generally contain from two (2) to twenty (20) amino acids. The term peptide also includes molecules that are commonly referred to as polypeptides, which generally contain from twenty (20) to fifty amino acids (50). The term peptide also includes molecules that are commonly referred to as proteins, which generally contain from fifty (50) to three thousand (3000) amino acids. The amino acids of the peptide may be L-amino acids or D-amino acids. A peptide, polypeptide or protein may be synthetic, recombinant or naturally occurring. A synthetic peptide is a peptide that is produced by artificial means in vitro.

As used herein, the term “barcode” refers to a molecule providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A “nucleic acid barcode” refers to a nucleic acid molecule of 2 to 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases). A “peptide barcode” or “amino acid barcode” refers to a sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids.

A specific peptide barcode can be distinguished from other peptide barcodes by having a different length, sequence, or other physical property (for example, hydrophobicity). A barcode can be an artificial sequence or a naturally occurring sequence. In certain embodiments, each barcode within a population of barcodes is different. In other embodiments, a portion of barcodes in a population of barcodes is different, e.g., at least 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc.

The term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs comprising natural or synthetic modifications of the sugar, nucleobase, and/or backbone structure. A nucleic acid molecule includes DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include xeno nucleic acid (XNA), bridged nucleic acid (BNA), glycol nucleic acid (GNA), peptide nucleic acids (PNAs), yPNAs, morpholino polynucleotides, locked nucleic acids (LNAs), threose nucleic acid (TNA), 2′—O-Methyl polynucleotides, 2′—O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a yPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

The phrase “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Detectable labels include any labels that can be utilized and are compatible with the provided polypeptide analysis assay format and include a bioluminescent label, a biotin/avidin label, a chemiluminescent label, a chromophore, a coenzyme, a dye, an electro-active group, an electro-chemiluminescent label, an enzymatic label, a fluorescent label, a latex particle, a magnetic particle, a metal, a metal chelate, a phosphorescent dye, a protein label, a radioactive element or moiety, and a stable radical. In provided embodiments, fluorescent labels are preferred.

Direct and indirect attachments (e.g., of a detectable label to an oligonucleotide or other substance) can include covalent bonds or non-covalent interactions. Covalent bonds include the sharing of electrons in a chemical bond. Non-covalent interactions include dispersed electromagnetic interactions such as hydrogen bonds (such as occurs between paired strands of nucleic acids), ionic bonds, van der Waals interactions, and hydrophobic bonds.

“Fluorescence” refers to the emission of visible light by a substance that has absorbed light of a different wavelength. In some embodiments, fluorescence provides a non-destructive means of tracking and/or analyzing biological molecules based on the fluorescent emission at a specific wavelength. Proteins (including antibodies), peptides, nucleic acid, oligonucleotides (including single stranded and double stranded primers), and so forth may be “labeled” with any of a variety of extrinsic fluorescent molecules referred to as fluorophores. Isothiocyanate derivatives of fluorescein, such as carboxyfluorescein, are an example of fluorophores that may be conjugated to proteins (such as antibodies for immunohistochemistry) or nucleic acids. In some embodiments, fluorescein may be conjugated to nucleoside triphosphates and incorporated into nucleic acid probes (such as “fluorescent-conjugated primers”) for in situ hybridization.

The terms “individual” or “subject” include birds (e.g., chickens, ducks, geese, turkeys, quail, songbirds, and so forth), other non-mammalian vertebrates (e.g., as fish), and mammals (e.g., mice, rats, rabbits, and other rodents; cats and other felines; dogs and other canines; other domesticated animals; pigs, cows, oxen, sheep, goats, horses, and other livestock animals; monkeys and other non-human primates). In certain embodiments, the individual or subject is a human.

The term “linker” refers to one or more of a nucleotide, a nucleotide analog, an amino acid, a peptide, a polypeptide, a polymer, or a non-nucleotide chemical moiety that is used to join two molecules to each other. A linker may be used to join a nucleic acid (such as a PSS) with an amino acid (such as an amino acid from a donor peptide, DP), and so forth. In certain embodiments, a linker joins two molecules via enzymatic reaction or chemistry reaction (e.g., click chemistry).

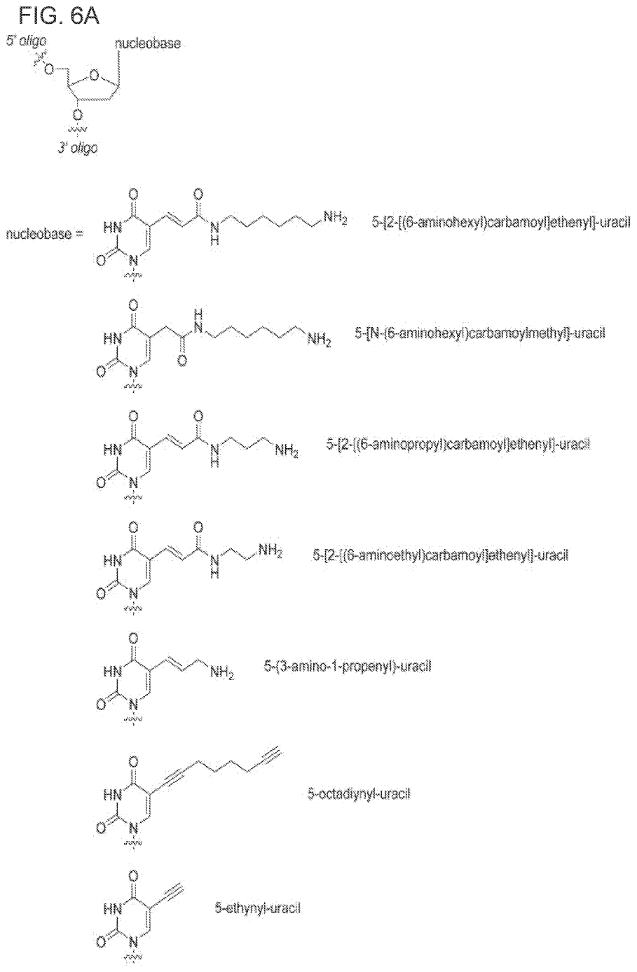

Examples of linkers for attachment amino acids to polymer backbone (such as a nucleic acid backbone) in a PROSE construct/hybrid molecules are shown in FIGS. 6A-6B. The shown amines, alkynes, azides are exemplary of functional groups that can be used, for instance, to link the NTAA to the PROSE construct via an ISOTHIOCYANATE (ITC) moiety. The length of the linker between the polymer and the amino acid can be 0 atoms up to numerous atoms. The longer the linker is, the more degrees of freedom the amino acid will have during translocation through the nanopore, which may increase variability in the recorded signal from the nanopore as the amino acid passing through the “read head” constriction within the pore.

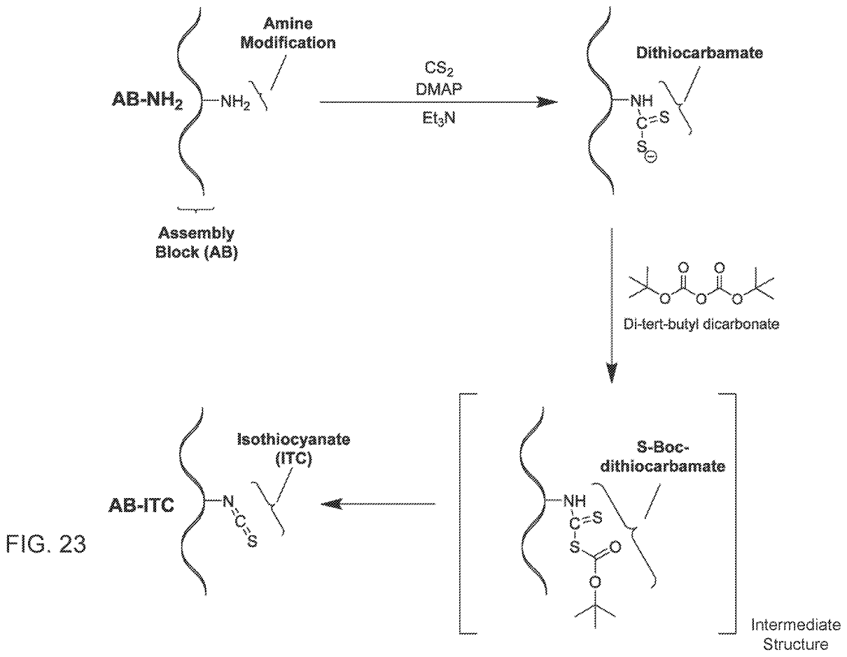

The ITC can be introduced either to the PROSE construct via direct amine conversion (FIG. 23), click chemical reactions, or other coupling reactions.

Alternatively, an ITC-containing molecule containing a secondary reactive group can first be attached to the NTAA and then the secondary reactive group can then be attached to a modification on the PROSE construct via click chemical reaction or other coupling reactions.

The linker is represented in Formulas I-XX herein as “L”. It is a linker group linking the structure that follows it in the formula to: adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer such as C3 spacer, spacer9, spacer18 spacers (e.g., available commercially at Integrated DNA Technologies (Coralville, IA), e.g., idtdna.com/site/catalog/modifications/category/6) or longer similar linkers; or to a non-natural or synthetic nucleobase or backbone structure; or to any structure contained within the definitions of “nucleic acid molecule” or “polynucleotide” herein. The linker can include longer spacers or non-nucleic acid residues between the amino acid SCD and the nearest nucleotide, thus having a generic structure of: NA-unilink-NA and NA-spacer9-unilink-spacer18-NA, for instance, where NA=nucleic acids. L can also be viewed as the structure resulting from a reactive functionality on the AB or hybrid polymer and a reactive functionality on the NTAA (or CTAA) of the donor peptide or resultant SCD. The structure indicated by “L” includes any structures or spacers included in the creation of the reactive functionalities. The linker structure includes any structures that were necessary to create reactive functionalities, such as from a canonical nucleotide to a N-terminal amino acid.

It is contemplated that linkers may be a chemical moiety that joins the single-stranded natural or synthetic biopolymer to the structure that follows the symbol “L” as illustrated herein, such as —(C₁-C₁₂ alkyl)-, as well as:

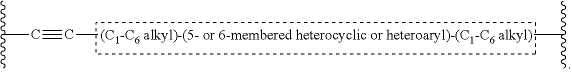

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (see e.g., Service, Science 311:1544-1546, 2006).

As used herein, “single molecule sequencing” or “third generation sequencing” refers to next-generation sequencing methods wherein reads from single molecule sequencing instruments are generated by sequencing of a single molecule, generally a molecule of DNA. Unlike next generation sequencing methods that rely on amplification to clone many DNA molecules in parallel for sequencing in a phased approach, single molecule sequencing interrogates single molecules (e.g., of DNA) and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

The term “sample” refers to anything which may contain an analyte (e.g., a protein or peptide) for which an analyte assay (e.g., detecting, quantifying, and/or sequencing) is desired. The term “sample” can include a solution, a suspension, liquid, powder, a paste, any of which may be aqueous or non-aqueous, or any combination thereof. The sample may be a biological sample, such as a biological fluid or a biological tissue, or individual cell(s). Examples of biological fluids include urine, blood, plasma, serum, saliva, semen, stool, sputum, cerebral spinal fluid, tears, mucus, amniotic fluid, and the like. Biological tissues are aggregate of cells, usually of a particular kind (or a mixture of two or more kinds) together with their intercellular substance that form one of the structural materials of a human, animal, plant, bacterial, fungal, or viral structure, including connective tissue, epithelium, muscle tissue, and nerve tissues. Examples of biological tissues also include organs, tumors, lymph nodes, and arteries. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include acylation, acetylation, alkylation (including methylation), biotinylation, butyrylation, carbamylation, carbonylation, C-terminal amidation, deamidation, deiminiation, diphthamide formation, disulfide bridge formation, eliminylation, farnesylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, and ubiquitination. A post-translational modification includes modifications of the amino terminus and/or the carboxyl terminus of a peptide. Modifications of the terminal amino group include des-amino, N-lower alkyl, N-di-lower alkyl, and N-acyl modifications. Modifications of the terminal carboxy group include amide, lower alkyl amide, dialkyl amide, and lower alkyl ester modifications (e.g., wherein lower alkyl is C₁-C₄ alkyl). A post-translational modification also includes modifications, such as those described above, of amino acids falling between the amino and carboxy termini. The term post-translational modification can also include peptide modifications that include one or more detectable labels.

The term “proteome” includes the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof.

Proteomics is the study of a proteome. Thus, the term “proteomics” encompasses quantitative analysis of the proteome within cells, tissues, and bodily fluids, and the corresponding spatial distribution of the proteome within the cell and within tissues. Additionally, proteomics studies include the dynamic state of the proteome, which is continually changing in time as a function of biology and defined biological or chemical stimuli.

In embodiments provided herein, the term “sample” includes any material that contains one or more polypeptides. The sample may be a biological sample, such as animal or plant tissue, biopsy, organ, cell(s), membrane vesicles, plasma membranes, organelles, cell extracts, secretions, urine or mucous or other secretion, tissue extracts or other biological specimens both natural or synthetic in origin. The term sample also includes single cells, organelles or intracellular materials isolated from a biological specimen, or viruses, prions, bacteria, fungus or isolates therefrom. The sample may also be an environmental sample, such as a water sample or soil sample, or a sample of any artificial or natural material, that contains one or more polypeptides.

The term “side chains” or “R” (or R group) refers to unique structures attached to the alpha carbon (attaching the amine and carboxylic acid groups of the amino acid) that render uniqueness to each type of amino acid. R groups have a variety of shapes, sizes, charges, and reactivities, such as Charged Polar side chains, either positively or negatively charged, such as lysine (+), arginine (+), Histidine (+), aspartate (−) and glutamate (−), amino acids can also be basic, such as lysine, or acidic, such as glutamic acid; Uncharged Polar side chains have Hydroxyl, Amide, or Thiol Groups, such as Cysteine having a chemically reactive side chain, i.e. a thiol group that can form bonds with another Cysteine, Serine (Ser) and Threonine (Thr), that have hydroxylic R side chains of different sizes; Asparagine (Asn), Glutamine (Gln), and Tyrosine (Tyr); Non-polar hydrophobic amino acid side chains include the amino acid Glycine; Alanine, Valine, Leucine, and Isoleucine having aliphatic hydrocarbon side chains ranging in size from a methyl group for alanine to isomeric butyl groups for Leucine and Isoleucine. Methionine (Met) has a thiol ether side chain, Proline (Pro) has a cyclic pyrrolidine side group. Phenylalanine (with its phenyl moiety) (Phe) and Tryptophan (Trp) (with its indole group) contain aromatic side groups, which are characterized by bulk as well as non-polarity.

Signposts: Modification(s) within the PROSE construct that augment the measured signal readout that can include modifications within the assembly blocks (ABs), changes in the polymer backbone, and post-PROSE modifications of the TC carboxyl groups.

As used herein, the terms “solid support”, “solid surface”, or “solid substrate”, or “sequencing substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof.

For example, when solid surface is a bead, the bead can include a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from 0.2 micron to 200 microns, or from 0.5 micron to 5 micron. In some embodiments, beads can be 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from 1 nm to 500 nm in diameter, for example, between 1 nm and 20 nm, between 1 nm and 50 nm, between 1 nm and 100 nm, between 10 nm and 50 nm, between 10 nm and 100 nm, between 10 nm and 200 nm, between 50 nm and 100 nm, between 50 nm and 150, between 50 nm and 200 nm, between 100 nm and 200 nm, or between 200 nm and 500 nm in diameter. In some embodiments, the nanoparticles can be 10 nm, 50 nm, 100 nm, 150 nm, 200 nm, 300 nm, or 500 nm in diameter. In some embodiments, the nanoparticles are less than 200 nm in diameter.

The most widely used reaction for the sequential analysis of N-terminal residue of peptides is the Edman degradation method (Edman et aL., Acta Chem. Scand. 4: 283-293, 1950). Edman degradation is a method of sequencing amino acids in a peptide (or protein) wherein the amino-terminal residue is labeled and cleaved from the peptide without disrupting the peptide bonds between other amino acid residues). In the Edman procedure, phenyl isothiocyanate (PITC) reacts quantitatively with the free amino group of a peptide to yield the corresponding phenylthiocarbamoyl peptide. On treatment with anhydrous acid, the N-terminal residue is split off as a phenylthiocarbamoyl amino acid; this leaves the remainder of the peptide chain intact. One aspect of the Edman degradation method is that the rest of the peptide chain (after removal of the N-terminal amino acid) is left intact for further cycles of this procedure; thus, the Edman method can be used in a sequential, iterative manner to identify a plurality of consecutive amino acid residues starting from the N-terminal end the peptide being analyzed.

PROSE: acronym for PROtein Sequencing by Expansion

PROSE construct (aka hybrid polymer): a polymeric structure with encoded amino acid side chain decorations (SCDs) with increased spacing relative to un-expanded proteins or peptides; can be thought of as a poly(assembly block(AB)-SCD) structure, or when intervening monomeric or polymeric sequences (IS) are used, a poly((AB-SCD)-IS) structure where AB may or may not carry an SCD.

AB: assembly block of the PROSE construct; typically contains one or more, but sometimes zero, modifications for conversion to an isothiocyanate (ITC) or isoselenocyanate group (ISC), for conversion to a click linker for subsequent addition of an ITC or ISC group, or conversion to a click linker for conjugation to an ITC or ISC that is already conjugated to the donor peptide or protein

IS: intervening monomeric or polymeric spacer sequence that, in some embodiments, is positioned between some or all adjacent assembly blocks (ABs)

χ: the polymeric seed strand (PSS) for assembly of the PROSE construct; this is the base or first assembly block (AB) to which additional ABs or intervening monomeric or polymeric sequences are added; this seed strand is the polymeric structure that can be connected to the donor peptide or protein and, in some embodiments, to a solid or semi-solid support via a multifunctional linker or other linking mechanism such as streptavidin-biotin binding; may include additional features such as a photocleavable linker for release of a completed PROSE construct, a unique molecular identifier (UMI) or barcode for reference or strand swapped, and others

PSS: It starts of as the definition of X (above). The AB blocks are added to the PSS during PROSE process. The PSS with AB block(s) (PSS+AB) may still be referred to as PSS because it is the proximal end to the next AB block

ITC: isothiocyanate chemical group

ISC: isoselenocyanate chemical group, an isothiocyanate analog

TU: the chemical group thiourea that is formed by the reaction of isothiocyanate with an amine

DP: donor peptide or protein; the peptide or protein from which SCDs are transferred to the PROSE construct

AA: amino acid

NTAA: N-terminal amino acid from or contained within a peptide or protein

CTAA: C-terminal amino acid from or contained within a peptide or protein

ICC: intermediate cyclical structure formed upon ligation of an assembly block (AB) to the PROSE construct under assembly; this structure is opened upon cleavage of the amino acid conjugated to the AB

SCD: amino acid side chain decoration borne by an assembly block (AB) of the PROSE construct

AB-AA: an assembly block (AB) of the PROSE construct containing a side chain decoration (SCD) in any form

AB-SCD: an assembly block (AB) of the PROSE construct containing a side chain decoration (SCD) in any form

AB-TZ: an assembly block (AB) of the PROSE construct containing a side chain decoration (SCD) in the thiazolinone form

AB-TC: an assembly block (AB) of the PROSE construct containing a side chain decoration (SCD) in the thiocarbamate form

AB-TH: an assembly block (AB) of the PROSE construct containing a side chain decoration (SCD) in the thiohydantoin form

TC: side chain decoration (SCD) in the thiocarbamate form

TH: side chain decoration (SCD) in the thiohydantoin form

AB-ATH: an assembly block (AB) of the PROSE construct containing a side chain decoration (SCD) in the alkylated thiohydantoin form, which is produced via the C-terminal degradation approach

ATH: side chain decoration (SCD) in the thiohydantoin form, which is produced via the C-terminal degradation approach

DP-TH: donor peptide or protein-thiohydantoin conjugate, which is produced via the C-terminal degradation approach

UMI: unique molecular identifier; an encoded identifier that can be readout by the detection scheme that that enables cataloging or indexing of each PROSE construct or each assembly block (AB) or intervening monomeric or polymeric sequence within the construct

PTM: post-translational modification

DL: docking linker; a multifunctional linker molecule that is used to link the donor peptide or protein (DP), the seed strand (X), and, in some embodiments, a solid or semi-solid support into an assembly

“N”, “N-1”, “N-2”, . . . position: amino acid (AA) positions within a peptide or protein relative to the N-terminus; the “N” position is occupied by the AA at the terminal position “N-1” is occupied by the adjacent AA located medial to the terminal position and so on

“C”, “C-1”, “C-2”, . . . position: amino acid (AA) positions within a peptide or protein relative to the C-terminus; the “C” position is occupied by the AA at the terminal position “C-1” is occupied by the adjacent AA located medial to the terminal position and so on

### (II) Overview

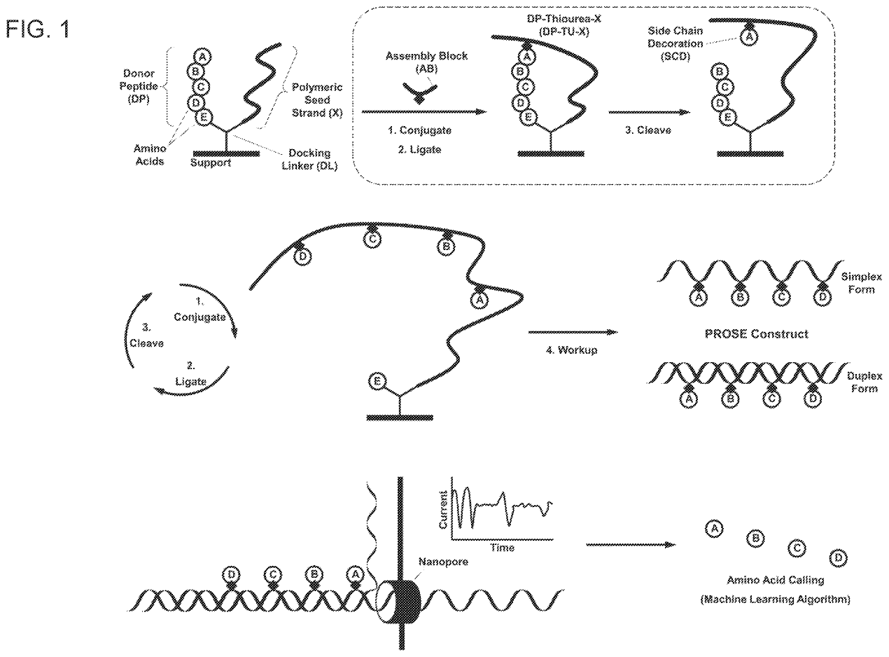

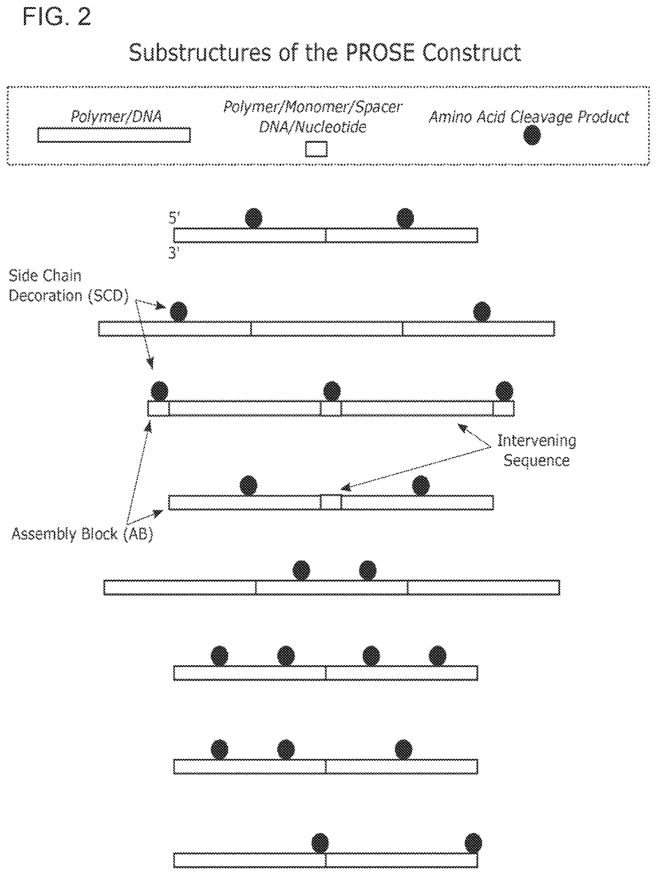

Provided herein are novel biochemical constructs and methods of de novo peptide sequencing via translocation through a nanopore (FIG. 1). Herein, the method is generally referred to as PROtein Sequencing by Expansion (“PROSE”) and the biochemical construct is generally referred to as the “PROSE construct”. The PROSE construct is a novel polymer within which the amino sequence of a peptide of arbitrary sequence is encoded (FIG. 2). The identity and relative position of each amino acid side chain from the donor peptide is retained, but the physical spacing between each consecutive side chain is increased to a user-defined value ranging from less than 1 nm to greater than 100 nm. This represents a substantial increase compared to the adjacent amino acid spacing of approximately 0.4 nm within a natural, unexpanded polypeptide chain. In the context of nanopore-based single molecule peptide sequencing, this expansion of the length scale between sequential amino acid sidechains drastically simplifies the occupancy problem by reducing or eliminating the signal contribution of adjacent amino acids.

For unexpanded peptides, it is nearly impossible to resolve the signal contribution of a single amino acid side chain as several amino acids flanking a given amino acid both N- and C-terminally will contribute substantially to the signal readout from that amino acid. However, once encoded within the PROSE construct, the signal from this amino acid is fully resolved from its neighboring residues. The flanking regions of the PROSE construct's polymeric backbone contribute to the signal readout from a given amino acid, but the exact structure of these flanking is user-defined and can be kept constant for each amino acid encoded within the construct such that it constitutes an expected and known baseline signal that can be subtracted. This simplifies the occupancy problem from N^{k} permutations for unexpanded peptides to just N¹ permutations for the PROSE construct, where N is the number of possible different amino acid side chains (including post-translational modifications) and k is the number of amino acids that simultaneously occupy the read head of the nanopore and contribute to the signal readout. Therefore, use of the PROSE construct for nanopore-based peptide sequencing simplifies the problem of exponential permutation scaling to one of linear scaling, which ultimately enables nanopore-based de novo peptide sequencing at single-amino acid resolution.

### (III) Enzymatic or Chemical Digestion

A protease, including proteolytic enzymes and peptidases, or a chemical agent will be used to digest proteins into component peptides. Skilled persons can select a protease from a database based on desired properties of the protease, including specificity to a particular amino acid or sequence of amino acids, known as the protease substrate. Curated proteolytic databases known in the art may include the MEROPS database (accessible at: ebi.ac.uk/merops/), the PANTHER database (accessible at: pantherdb.org), the BRENDA database (accessible at: brenda-enzymes.org), the TopFIND database (accessible at: topfind.clip.msl.ubc.ca), and the UniProt database (accessible at: uniprot.org). If a protease exhibits sufficiently high specificity toward a particular amino acid, a particular class or attribute of the side chain (e.g. aromatic, aliphatic, basic, acidic, size, functional group, etc.), a polypeptide sequence, or combination therein, then a protein will be cleaved by the protease in a predictable manner such that the specific identity or the class or attribute identity of certain amino acids within the component peptides will be known with a high level of confidence.

For example, if a person skilled in the art used the Homo sapiens serine protease, matriptase-2 (UniProt accession: Q81U80, MEROPS database ID: SO1.308), which cleaves the polypeptide backbone directly C-terminal to an arginine residue with near 100% specificity, then it would be known that the C-terminal residue in each component peptide formed through enzymatic digestion with matriptase-2 would be arginine. In a second example, if a person skilled in the art used the Homo sapiens metalloprotease PHEX peptidase (UniProt accession: P78562, MEROPS database ID: M13.091), which cleaves the polypeptide backbone directly N-terminal to an acidic, carboxyl-containing amino acid with near 100% specificity, then it would be known that the N-terminal amino acid in each component peptide would be either be a glutamic acid or aspartic acid residue. In a third example, a person skilled in the art can use cyanogen bromide (CAS registry number: 506-68-3, IUPAC: carbononitridic bromide), which cleaves the polypeptide backbone directly C-terminal to methionine residues with high specificity (Gross, “The cyanogen bromide reaction,” in Methods in Enzymology, vol. 11: Academic Press, 1967, pp. 238-255. doi.org/10.1016/S0076-6879(67)11029-X). Therefore, it would be known that the C-terminal residue in each component peptide would be methionine.

The choice of a selective enzyme or chemical agent for protein digestion also grants the benefit of providing an internal reference within the generated component peptides that can inform whether a PROSE construct that is shorter than expected (according to the number of workflow cycles performed) is due to incomplete reactions within the workflow or simply due to reaching the final residue of the donor peptide (DP). Choosing a one or more selective digestion agent results in knowledge of the identity of certain C-terminal amino acids, N-terminal amino, or a combination therein prior to performing the sequencing workflow, such that the identification of the corresponding side chain decoration (SCD) or sequence of SCDs is indicative of complete encoding of a given donor peptide into the PROSE construct. This is valuable information because digestion produces a broad mixture of peptides lengths. Therefore, without an internal reference indicating complete transferal of SCDs from a DP to the PROSE construct it would otherwise be difficult to ascertain why a construct is shorter than the expected length.

(IV) Blocking Chemical Reactivity with Protecting Groups

Before or after enzymatic or chemical digestion, reactive side chains reactive chemical groups of the protein or peptides, respectively, can be blocked with protecting groups to prevent side reactions and degradative processes from occurring in the subsequent steps of the PROSE workflow. These reactive and/or sensitive groups include amino-, carboxyl-, sulfhydryl-, hydroxyl-, amido-, and guanidino-containing side chains as well as other potentially reactive groups from canonical, non-canonical, synthetically modified, and post-translationally modified side chains.

Protecting groups and their reactions are well-understood and will be chosen based on compatibility with downstream chemical reactions (Isidro-Llobet et al., Chem Rev 109(6):2455-504, 2009. doi.org/10.1021/cr800323s; Kocienski, Protecting Groups, 3rd ed. Georg Thieme Verlag, 2005. ISBN 9781588903761). Protecting groups can be added before enzymatic cleavage, which might require sacrificing the N- and C-terminal component peptides as the N-terminal amine and C-terminal carboxylic acid of the protein might also be protected, which would render them inert to the downstream reactions of sequencing protocol. Protecting groups can be added after enzymatic digestion, but this requires use of specific protecting agents that would not protect the N- or C-terminal functional groups of the component peptides. In certain cases, a protecting group that protects the epsilon amine of lysine would also protect the alpha amine of the N-terminus. If the protecting group was an ITC derivative, then subsequent Edman cleavage conditions would revert the N-terminal back to a free amine. The lysine side chain would remain protected/blocked.

In addition, protecting groups can be used to modulate the nanopore readout of different amino acid side chains. Protecting group schemes involving the selective addition of modifications including small, large, aromatic, charged, metallic, trans-metallic, oligomeric, polymeric, particulate, fluorophore, chromophore, other chemical moieties, or combinations therein to specific side chains can be used strategically to alter or enhance the dynamic range of detection. For example, these modifications will alter characteristic electrical readouts (the “squiggles”) of different amino acid side chain passing through the read head(s) of a nanopore, which may be advantageous for amino acid identification by enhancing the signal differential between certain side chains. Peptides can be identified by just these modified side chains if the read length is sufficiently long to generate a pattern that can be aligned to a peptide database. In a PROSE construct, the distance between the modified sidechains can also be determined by knowing the translocation speed or incorporating “signposts” which can include detectable modification(s) within the PROSE construct that augment the measured signal readout and/or background signal that can include modifications within the assembly blocks (ABs), changes in the polymer backbone, and post-PROSE modifications of the TC carboxyl groups.

Should certain SCDs be difficult to resolve from the background signal, then these post-PROSE modifications could enhance the signal contributed by a given SCD passing through the nanopore. In this case, such a modification would ensure that all amino acids are detectable by the nanopore, and a subset of these amino acids would then be identifiable based on the modifications. The larger modifications could also enable the use of larger diameter biological pores or synthetic solid state nanopores due to the increased signal as the modifications pass through the pore.

### (V) Oligonucleotide Composition

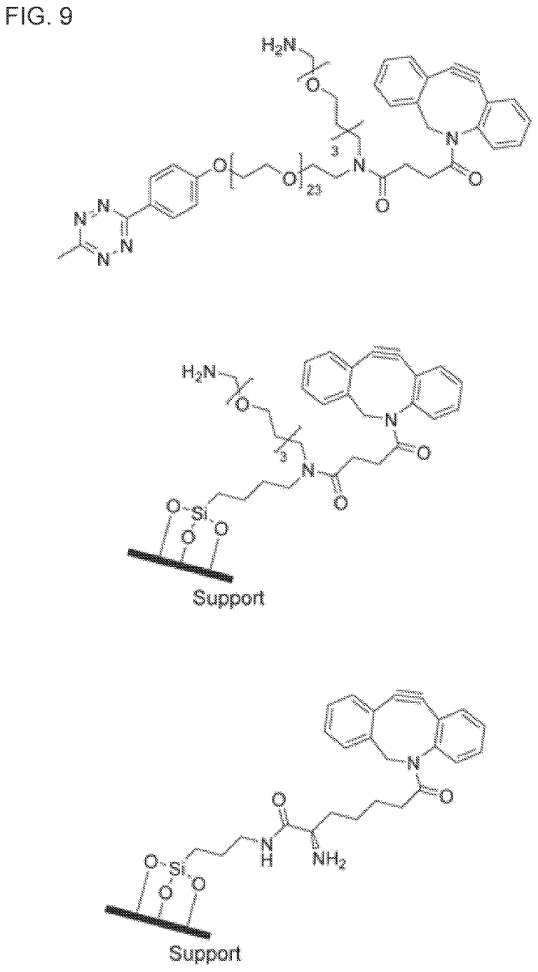

The chemical conditions of the workflow are factored into the design of oligonucleotides to minimize degradation processes including nucleobase removal (“debasing”) and disruption of the polymeric backbone of the oligonucleotides (“strand scission”). Purine nucleobases within oligonucleotides, such as the canonical adenine and guanine nucleobases, are particularly susceptible to debasing (“depurination”) when exposed to harsh chemical conditions (FIG. 10). These conditions include highly acidic or basic environments. Pyrimidine nucleobases contained within oligonucleotides; such as the canonical thymine, cytosine, and uracil nucleobases (collectively referred to with the degenerate pyrimidine code, “Y”); are less susceptible to debasing (“depyrimidination”) than purine nucleobases, but this process can occur with harsher conditions. Once debasing occurs, the abasic sites within the oligonucleotides are susceptible to strand scission, which results in a break in the oligonucleotide backbone directly 3′ to the formed abasic site. Variables including pH, salt content, acid or base concentration, exposure time, temperature, redox environment, and solvent composition can influence these processes (FIG. 10). Under conditions where debasing might occur, the inclusion of reducing agents can inhibit subsequent strand scission from occurring.

In one example of the PROSE workflow, the oligonucleotides present during the highly acidic Edman degradation steps are designed to contain only pyrimidine nucleobases. This design minimizes the degradative processes of debasing and strand scission (TABLE 1).

TABLE 1
Example set of oligonucleotides used in the PROSE workflow

| | Name | | |
|---|---|---|---|
| ID# | SEQ ID | Description and Features | Sequence |
| P001 | X | Seed strand for PROSE | 5′ [DBCO]T[T_{BIO}] T TTT TTT TTT TTT |
| | SEQ ID | construct assembly; 5′ | TTT TTT [PCL_{2NB}] TTT TCC CCT CCT |
| | NOs: 4 | dibenzocyclooctyne [DBCO], | TCT TTT TTT CTC CTC TCT CCT |
| | and 62 | internal biotin modified | CCT 3′ |
| | | thymine [T_{BIO}], internal 2- | |
| | | nitrobenzyl photocleavable | |
| | | linker [PCL_{2NB}] | |
| P003 | AB₁ | Assembly block 1; 5′ | 5′ [P] CTC TCC TCC TTC TCT |
| | SEQ ID | phosphorylation [P], internal | TT[T_{C6N}] TTT CTC TTC TCC CTC |
| | NO: 5 | hexyl-amine modified thymine | TCC 3′ |
| | | [T_{C6N}] | |
| P005 | AB₂ | Assembly block 2; 5′ | 5′ [P] TTT TCC TCT CCT TCT |
| | SEQ ID | phosphorylation [P], internal | TT[T_{C6N}] TTT CTC CTC TCT CCT |
| | NO: 6 | hexyl-amine modified thymine | CCT 3′ |
| | | [T_{C6N}] | |
| P007 | P | Priming and UMI sequence; 5′ | 5′ [P] CTC TCC TCC TTC TCT TVN |
| | SEQ ID | phosphorylation [P], internal | NNN NVT AGA TCG GAA GAG CGT |
| | NO: 7 | UMI code with degenerate | CGT 3′ |
| | | bases V (A, C, G) and N | |
| | | (A, C, G, T) | |
| P002 | Spl(X-AB₁) | Splint for enzymatic ligation of | 3′ GAG GAG AGA GGA GGA GAG |
| | Spl(AB₂-P) | X with AB₁ and AB₂ with P | AGG AGG AAG AGA 5′ |
| | SEQ ID | | |
| | NO: 8 | | |
| P004 | Spl(AB₁- | Splint for enzymatic ligation of | 3′ GAG AAG AGG GAG AGG AAA |
| | AB₂) | AB₁ with AB₂ | AGG AGA GGA AGA 5′ |
| | SEQ ID | | |
| | NO: 9 | | |
| P014 | AB_{1(V2)} | Assembly block 1, version 2; | 5′ [P] CTC TCC TCC TTC TCT |
| | SEQ ID | 5′ phosphorylation [P], | TT[T_{C2N}] TTT CTC TTC TCC CTC |
| | NO: 10 | internal ethyl-amine modified | TCC 3′ |
| | | thymine [T_{C2A}] | |
| P015 | AB_{2(V2)} | Assembly block 2, version 2; | 5′ [P] TTT TCC TCT CCT TCT |
| | SEQ ID | 5′ phosphorylation, internal | TT[T_{C2N}] TTT CTC CTC TCT CCT |
| | NO: 11 | ethyl-amine modified thymine | CCT 3′ |
| | | [T_{C2N}] | |
| P030 | Spl(X-AB₁) | Alternative splint for | 3′ AAA GAG GAG AGA GGA GGA |
| | Spl(AB₂-P) | enzymatic ligation of X with | GAG AGG 5′ |
| | SEQ ID | AB₁ and AB₂ with P | |
| | NO: 14 | | |
| P031 | Spl(AB₁- | Alternative splint for | 3′ A GAG AAG AGG GAG AGG AAA |
| | AB₂) | enzymatic ligation of AB₁ with | AGG A 5′ |
| | SEQ ID | AB₂ | |
| | NO: 15 | | |
| P020 | Primer-1 | Priming with specific UMI | 5′ [P] CTC TCC TCC TTC TCT |
| | SEQ ID | sequence; 5′ phosphorylation | TATGCGATCGGAAT AGA TCG GAA |
| | NO: 16 | [P] | GAG CGT CGT 3′ |
| P021 | Primer-2 | Priming with specific UMI | 5′ [P] CTC TCC TCC TTC TCT |
| | SEQ ID | sequence; 5′ phosphorylation | TCCGGATTAGACGT AGA TCG GAA |
| | NO: 17 | [P] | GAG CGT CGT 3′ |
| P022 | Primer-3 | Priming with specific UMI | 5′ [P] CTC TCC TCC TTC TCT |
| | SEQ ID | sequence; 5′ phosphorylation | TCGTCATAGTGGAT AGA TCG GAA |
| | NO: 18 | [P] | GAG CGT CGT 3′ |
| P023 | Primer-4 | Priming with specific UMI | 5′ [P] CTC TCC TCC TTC TCT |
| | SEQ ID | sequence; 5′ phosphorylation | TGAGATAGGATGTT AGA TCG GAA |
| | NO: 19 | [P] | GAG CGT CGT 3′ |
| P024 | Primer-5 | Priming with specific UMI | 5′ [P] CTC TCC TCC TTC TCT |
| | SEQ ID | sequence; 5′ phosphorylation | TGGCAGCAATCCAT AGA TCG GAA |
| | NO: 20 | [P] | GAG CGT CGT 3′ |
| P025 | Primer-6 | Priming with specific UMI | 5′ [P] CTC TCC TCC TTC TCT |
| | SEQ ID | sequence; 5′ phosphorylation | TGCTATAATAGTGT AGA TCG GAA |
| | NO: 21 | [P] | GAG CGT CGT 3′ |
| P026 | Primer-7 | Priming with specific UMI | 5′ [P] CTC TCC TCC TTC TCT |
| | SEQ ID | sequence; 5′ phosphorylation | TATCAGTCAGAGTT AGA TCG GAA |
| | NO: 22 | [P] | GAG CGT CGT 3′ |
| P027 | Primer-8 | Priming with specific UMI | 5′ [P] CTC TCC TCC TTC TCT |
| | SEQ ID | sequence; 5′ phosphorylation | TACCGTTACGCCAT AGA TCG GAA |
| | NO: 23 | [P] | GAG CGT CGT 3′ |
| P028 | Primer-9 | Priming with specific UMI | 5′ [P] CTC TCC TCC TTC TCT |
| | SEQ ID | sequence; 5′ phosphorylation | TCCGAACGGCGCGT AGA TCG |
| | NO: 24 | [P] | GAA GAG CGT CGT 3′ |
| P029 | Primer-10 | Priming with specific UMI | 5′ [P] CTC TCC TCC TTC TCT |
| | SEQ ID | sequence; 5′ phosphorylation | TATCATCAGAGGAT AGA TCG GAA |
| | NO: 25 | [P] | GAG CGT CGT 3′ |

In a second example of the PROSE workflow, the oligonucleotides present during the highly acidic Edman degradation steps are designed to contain only thymine nucleobases. This design again minimizes the degradative processes of debasing and strand scission.

In other examples, the oligonucleotides could contain various combinations of the five canonical nucleobases and non-canonical, synthetic, and/or unnatural nucleobases with a canonical, partially modified, or fully modified backbone. The inclusion of purine nucleobases within oligonucleotides present during Edman degradation may require variations to the protocol. These variations may include partial or full substitution of strong Bronsted-Lowry acids for weak Bronsted-Lowry acids, partial or full substitution of strong Bronsted-Lowry acids for Lewis acids (Matsunaga et al., Anal Chem 68(17):2850-6, 1996. doi.org/10.1021/ac951253r), lower concentrations of acid, varying solvent conditions, inclusion of stabilizing synthetic modifications to the poly-nucleotide chain, inclusion of stabilizing cations (divalent cations, polycations, etc.), shorter reaction times, and lower temperatures. PROSE constructs that experience debasing events without strand scission are still functional for sequencing peptides. Sugar modifications that inhibit strand cleavage, such as LNA, may be advantageous.

These variations in reaction conditions can also be applied to the examples of oligonucleotides containing only pyrimidine nucleobases. Using milder conditions with these relatively stable oligonucleotides would enhance the safety factor of the experimental system against unintended oligonucleotide degradation. By further reducing stochastic depyrimidination, the fidelity of the PROSE construct after several cycles of the PROSE workflow can be enhanced.

As mentioned, the designed oligonucleotides can also contain modifications to the backbone, to the nucleobases, or a combination therein to improve hybridization stability (including locked nucleic acids (LNA)); increase flexibility (including fleximer nucleobases; Bardon et al., J Phys Chem A 109(1):262-72, 2005. doi.org/10.1021/jp046957w); provide universalizability for annealing (Romesberg et al., Curr Protoc Nucleic Acid Chem Chapter 1 Unit 15, 2002. doi.org/10.1002/0471142700.nc0105s10); and/or provide a means of chemical conjugation to a peptide, ligation to oligonucleotides, or attachment to various substrates. In one example, unnatural nucleobases containing an amino acid moiety can also be used.

In one example, one or more amine modifications, such as those shown in FIGS. 6A-6B, can be included within the assembly block (AB) oligonucleotides that are ultimately ligated together or with intervening monomeric or polymeric sequences (ISs) to form the amino acid side chain decorated (SCD) PROSE construct (FIG. 2). To enable the Edman degradation reaction, these amine modifications can be directly or indirectly converted to isothiocyanato (ITC) groups (Edman, Acta Chemica Scandinavica 4(7):283-293, 1950). The direct conversion involves reaction with carbon disulfide to yield the AB-ITC. The indirect conversion first converts the amine to a click reagent for subsequent addition of an ITC group via a bifunctional molecule consisting of the click complement and an ITC. Alternatively, this bifunctional molecule can first be reacted with the donor peptide or protein and the AB can be clicked to this pre-conjugated ITC.

### (VI) Oligonucleotide Length

Designed oligonucleotides must also be of adequate size for all processes. First, ligation of DNA can be performed blunt-ended or with an overlap of 1 or more bases, but longer sections of overlap can increase the efficiency of the ligation for commonly used ligases, such as T4 DNA ligase (Horspool et al., BMC Res Notes 3291, 2010. doi.org/10.1186/1756-0500-3-291). Furthermore, commonly used ligases often require at least 4 or more bases flanking either end of the ligation site for ligation to occur, and longer flanking regions of 5, 6, or more bases can substantially increase ligation efficiency. Therefore, four (4) bases is the lower length limit for the assembly block oligonucleotide (“AB”) when enzymatic ligation is used. Chemical ligation techniques could further reduce the required length of the oligonucleotide.

Use of shorter oligonucleotides can reduce the spacing between adjacent amino acid side decorations (“SCDs”) on the PROSE construct to increase the sequencing throughput, but as the side chain spacing is reduced to approximately the length of the nanopore read head, then the signal of each side chain will no longer be fully resolved from the neighboring residues. Full signal resolution of each side chain is not strictly required as machine learning methods can still assign sequences if the set of possible sequence permutations remains low. Limiting the strong, simultaneous signal contributions to fewer than three amino acids ensures a greatly simplified occupancy problem and, thus, easier single-amino acid resolution sequence assignment.

For example, if for a given nanopore the majority of the signal is contributed by a three (3) nucleotide k-mer (one source indicates that Oxford Nanopore Technologies claims this for their six (6) nucleotide read head occupancy R9 nanopore (Rang et al., Genome Biol 19(1):90, 2018. doi.org/10.1186/s13059-018-1462-9), then amino acid decorations (SCDs) within the PROSE construct should have at least one nucleic acid monomer, or a chemical linker consisting of a structure with a contour length comparable to that of one DNA nucleotide, located between it and the next amino acid amino acid decoration (SCD) to achieve 441 possible permutations, assuming these two decorations are canonical side chains. If two nucleic acid monomers, or a chemical linker consisting of a structure with a contour length comparable to that of two DNA nucleotides, were present between each subsequent amino acid decoration (SCD), then 21 possible permutations of canonical side chains would be possible.

The assembly block oligonucleotides (AB) used for sequencing, should also not be excessively such that they interfere with downstream processes, including ligation and nanopore sequencing. The upper limit of length is primarily a practical limit, rather than a technical limit. If longer oligonucleotides are used, then the information density of amino acid side chains encoded into the PROSE construct is reduced. This means that a greater length of the PROSE backbone must pass through the nanopore before each subsequent amino acid modification is read during sequencing, which represents a reduction of peptide sequencing throughput. Therefore, shorter oligonucleotides allow for increased amino acid side chain density on the construct, which effectively increases peptide sequencing throughput.

If longer ABs are used, then the overall length of the final PROSE construct is also increased in proportion to the number of amino acid decoration (SCD) and ligation cycles performed. With each cycle performed, increasingly long portions of the partially-assembled construct must be subsequently splinted, which may increase the stochasticity of annealing and ligation. Ultimately, while longer oligonucleotides, even those as long as 100—to 200-mers can be used, it is practical to use shorter (e.g., no more than 95, no more than 90, no more than 85, no more than 80, no more than 75, no more than 70, no more than 65, no more than 60, no more than 55, no more than 50, no more than 45, no more than 40, no more than 35, no more than 30, no more than 25, no more than 20, or shorter than 20) oligonucleotides so as to increase the amino acid information density of the PROSE construct.

### (VII) Thiocarbamate and Thiohydantoin Formation

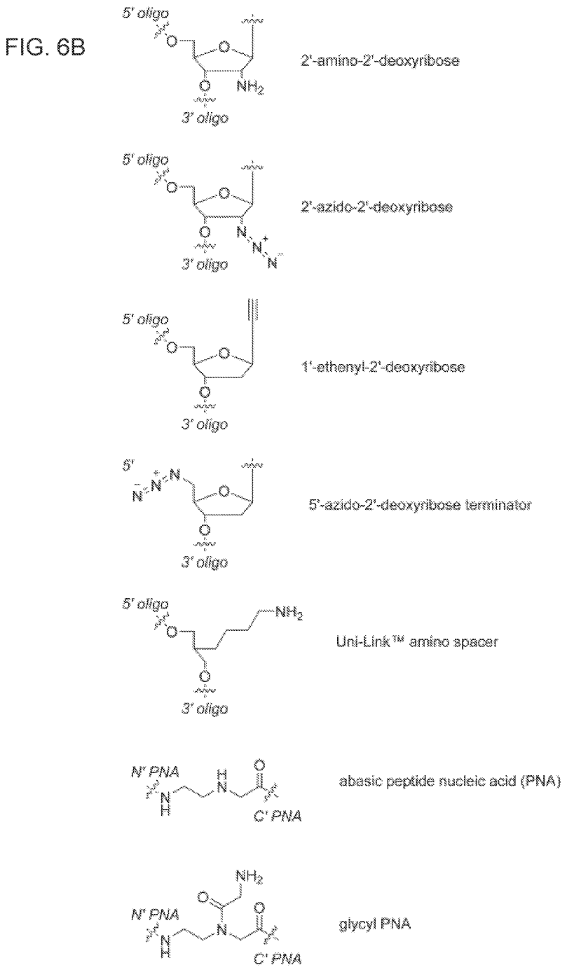

Edman degradation of the AB-TU-Peptide conjugate (where TU refers to thiourea) can yield three main oligonucleotide-amino acid (“AB-AA” or “AB-SCD”) products following cleavage of the N-terminal amino acid (NTAA): oligonucleotide-thiazolinone (“AB-TZ”), oligonucleotide-thiocarbamate (“AB-TC”), or oligonucleotide-thiohydantoin (“AB-TH”) (FIG. 7). The AB-TZ product is unstable under certain conditions and, typically, under those conditions is rapidly hydrolyzed into the AB-TC product. The AB-TZ product is thus prone to hydrolysis, and may hydrolyze to TC. Under aqueous conditions TZ can readily hydrolyze to TC, but under anhydrous conditions within non-nucleophilic solvent, one can isolate and characterize TZ. The AB-TH product is more thermodynamically stable than the AB-TC product and is formed via a cyclization reaction of the AB-TC thiocarbamate group. This cyclization is typically driven by applied heat under acidic conditions (Bhown et al., in Advanced Methods in Protein Microsequence Analysis, B. Wittmann-Liebold, J. Salnikow, and V. A. Erdmann Eds. Berlin, Heidelberg: Springer Berlin Heidelberg, 1986, pp. 208-218. ISBN 9783642715341. doi.org/10.1007/978-3-642-71534-1_17; Edman, Nature 177(4510):667-668, 1956. doi.org/10.1038/177667b0; Richards et al., Methods Enzymol 25314-26, 1972. doi.org/10.1016/S0076-6879(72)25027-3). Furthermore, these three structures can be interconverted as needed using known conditions (Farnsworth et al., Anal Biochem 215(2):200-10, 1993. doi.org/10.1006/abio.1993.1576).

Within each of these AB-AA products, the original side chain (or a protected derivative) of the NTAA is retained, which encodes the identity of the NTAA into the oligonucleotide sequence. After cleavage of the original “N” position amino acid NTAA from the peptide, the “N-1” position amino acid constitutes a newly-formed N-terminus and can then be referred to as the NTAA.

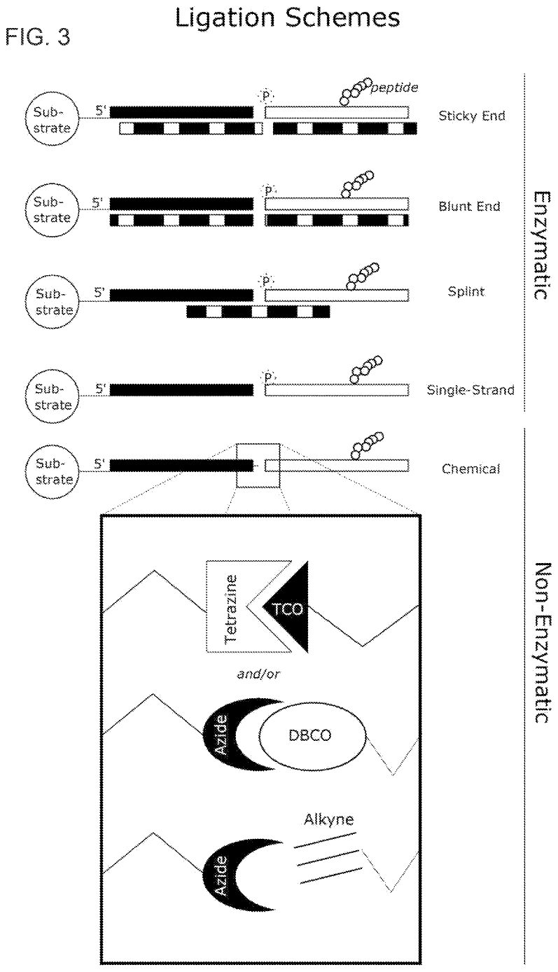

Each subsequent cycle of the PROSE workflow also encodes the identify of each subsequent amino acid within the polypeptide chain by retention of the respective side chain. The relative position of each amino acid within the original polypeptide chain is also encoded within the poly-nucleotide PROSE construct. This original positional information is retained because each subsequently-formed AB-TU-peptide conjugate is concatenated to the already formed portion of the PROSE construct via chemical or enzymatic ligation (FIG. 3). The assembled PROSE construct resembles a string of colored light bulbs for which, in this analogy, the wires between the light bulbs represent the oligonucleotide and the color of the light bulbs represent the identity of the amino acid side chains encoded in thiocarbamate or thiohydantoin form and the relative position of each light bulb in the string corresponds directly to the relative positioning of each amino acid within the original polypeptide structure.

Since this method uses the predictable chemistry of the Edman degradation to cleave the backbone of the peptide C-terminal to the NTAA, amino acid side chains of arbitrary identity can be encoded into the oligonucleotide sequence of the PROSE construct. Furthermore, the workflow is agnostic to the chemical identity of the side chains that will be encoded. That is, canonical, post-translationally modified, synthetically-modified, protected, unnatural, and non-canonical side chains can all be encoded within the PROSE construct without requiring prior knowledge of the chemical identity of each side chain. To avoid unpredictable side reactions, standard protecting groups can first be applied to potentially reactive side chains of the peptide or parent protein. Still, no prior knowledge of the sequence is required because regardless of the presence of the target functional group(s) of the protecting group reactions in the polypeptide or protein, the respective protecting group reactions can still be performed. This can also be referred to as an unbiased approach.

While it can be favorable to have all PROSE construct side chain decorations (SCDs) in a single form, either the thiocarbamate (TC) or thiohydantoin (TH) form, it is not strictly necessary as each form will have a characteristic readout when it passes through the nanopore. However, the disadvantage of a TC and TH mixture is that it doubles the set of possible SCDs. For example, the 21 canonical amino acid side chains would have a set of 42 TC and TH forms.

### (VIII) Ligation Schemes

PROSE constructs can be assembled by sequential ligation of DNA/polymer blocks with amino acid cleavage products (AB-TH, AB-TC). Ligation of these blocks can proceed through various enzymatic or chemical ligation schemes (FIG. 3): Sticky End Ligation, where strands are hybridized with base overhangs (1-10 bases, including T/A ligation); blunt end ligation where strands are hybridized leaving a blunt end between blocks; Splint ligation, where a single hybrid strand is used to join both polymer blocks; single-strand ligation, where a single-strand capable ligase is used to covalently ligate single-strand DNA; and non-enzymatic chemical ligation, where small molecules are used to covalently or non-covalently link the single-strand polymers using click chemistry compliments or other known reactions. Click chemistry is a reaction between an azide and an alkyne yield to yield a disubstituted 1,2,3-triazole. The chemistry is based on copper catalysis. Click chemistry reactions are well known (e.g., Scinto et al., Nat Rev Methods Primers 1:30, 2021, doi.org/10.1038/s43586-021-00028-z; Hein et al., Pharm Res 25, 2216-2230, 2008, doi.org/10.1007/s11095-008-9616-1; Seo et al., J Org Chem 68(2):609-612, 2003, DOI: 10.1021/jo026615r).

Regardless of the chemistry used for strand ligation, the final construct may contain various repeating elements (FIG. 2). These may repeat contiguously or with substantial spacing and may contain one or more amino acid cleavage products. One primary example has been produced in the current workflow, where all amino acid blocks are ligated sequentially to the preceding block and contain a unique or identical DNA sequence to that preceding block. These block sequences, whether used for peptide conjugation or not, may be used at any position in the workflow, which may be trivially dependent on complementation to the reverse compliment strands or chemicals used during the ligation.

Other various repeating units of the final construct can be created (FIG. 2). One example is using a spacing strand without peptide conjugation capabilities to increase the length of the construct, increase spacing of amino acid cleavage products, insert unique-molecular identifiers, insert barcodes, or increase the complexity of the oligo sequence. Another example of a repeating unit would be to use short blocks for conjugation (as small as a single molecule or nucleotide in any of its forms) and spacing polymers to adequately expand the amino acid cleavage products for sequencing. Another example would be to use the inverse of the latter to increase chemical ligation efficiency or insert complimentary or contrasting signals during sequencing. Another design could include multiple peptide attachment sites with one or more with protecting groups to ensure two or more amino acid cleavage products per block. This embodiment could be separated by spacing blocks to ensure a unique multi-amino acid signature during sequencing, ligated sequentially to generate dense and complex sequencing signals, or mixed with single amino acid cleavage blocks. With all of these block styles, the amino acid cleavage sites can reside at any place on the block. One example of an advantageous placement may be at the extremes of each block to increase efficiency in conjugation and cleavage. It is also possible to use any of these repeating units while mixing the placement of amino acid cleavage products on both main and complementing strands with the use of a unique molecular identifier (UMI) encoded in the strands.

### (IX) Barcoding and Unique Molecular Identifiers (UMIs)

To ensure peptide and protein counting capabilities and demultiplexing of reads, a unique molecular identifier (UMI) can be inserted in the final PROSE construct at the tail ends, or if useful, in between blocks. This can be combined with barcoding, which unlike a UMI, is a known sequence to separate information into grouped categories, such as for the purpose of multiplexing samples. This can be advantageous for spatial proteomics, single-cell proteomics, and other forms of selective grouping or analysis. Both UMI and barcoded regions can consist of canonical or non-canonical bases, or other synthetic modifications that may produce a specific electrical, optical, or enzymatic readout in downstream processing, such as sequencing.

Both UMI and barcoding strategies allow for sequencing error analysis and correction as well as missing block prediction. In one example, a cycle-specific barcode is ligated between blocks. In the event of a missing barcode, the peptide may be reconstructed by inference or omitted from in analysis after sequencing. Alternatively, the cycle barcode code be embedded in the PROSE assembly blocks (ABs).

Individual PROSE constructs may be ligated or concatenated together to form longer complexes for sequencing efficiency. An UMI/barcode in the X linker or end of the PROSE construct could delineate the start and/or end of each PROSE construct. A barcode added after concatenation could identify each sample or condition. This barcoding could be part of the PROSE process or as part of the library prep process.

### (X) Docking Linker (DL) Design

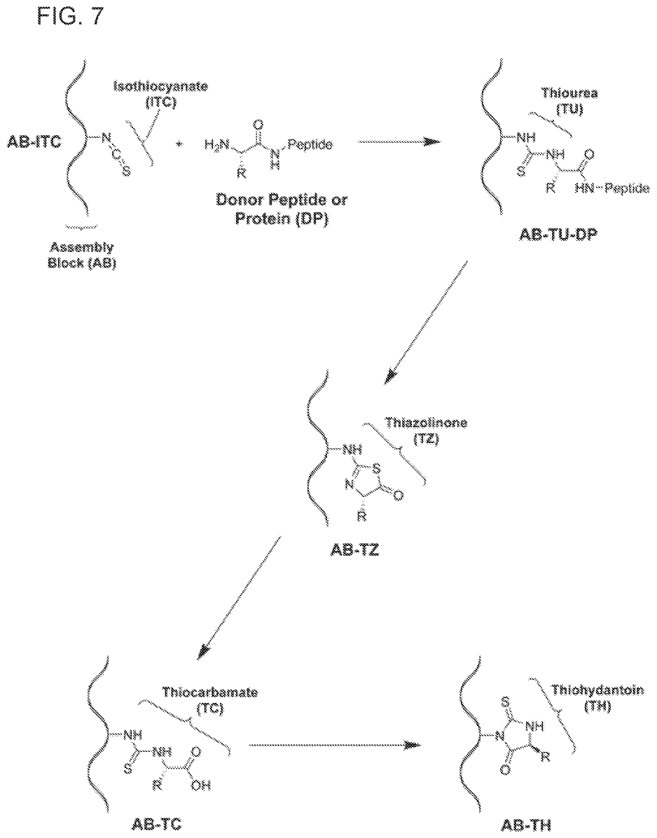

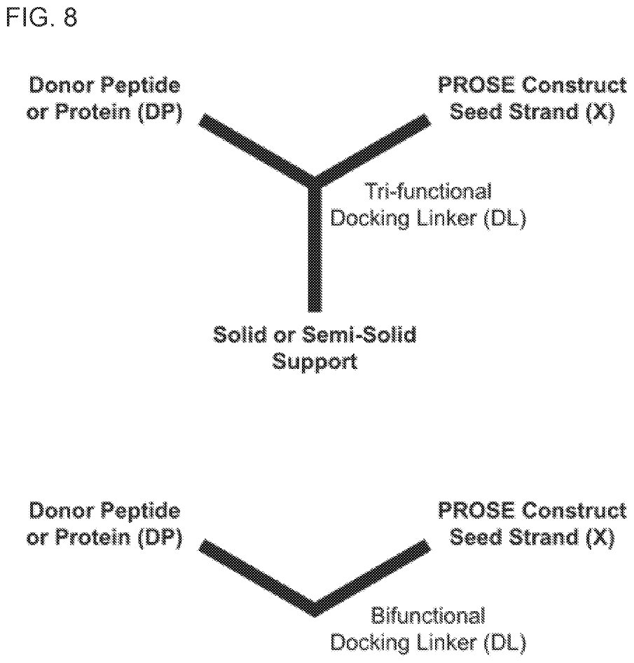

In some embodiments of the PROSE workflow, a multi-functional docking linker (DL) is used to attach molecules of interest, such as a peptide and a seed strand (X) for the PROSE construct, to a solid or semi-support (FIG. 8). Examples of these DLs are shown in FIG. 9. Whereas tri-functional DLs are useful for linking the following components into a single assembly: (1) a peptide, (2) a seed strand (PSS) for the PROSE construct, and (3) a solid or semi-solid support; bifunctional versions of these DLs can be used in variations of the PROSE workflow that do not include a solid or semi-solid support. The support could be 2D planar or 3D bead substrates, that could have different porosities, and that are comprised of glass, silica, silicon, hydrogel, or other polymer material. Attachment to the substrate could be covalent or noncovalent. Examples of covalent interactions, include silane, click, and acrylate based chemistries. Examples of noncovalent interactions include streptavidin/biotin, polyHis/NTA, Digoxigenin/anti-dig (ligand/antibody). Noncovalent, reversible, and/or cleavable schemes may be advantageous because some reactions may be faster and more efficient in solution, whereas washing steps are more efficient and simpler on a support.

These bifunctional DLs as well as other bifunctional DLs are useful for linking the following components into a single assembly: linking the following components into a single assembly: (1) a peptide and (2) a seed strand (X) for the PROSE construct. Examples of synthetic scheme for DL are described in “(XXV) Synthesis of Multi-Functional Docking Linkers (DLs)”

(XI) PROSE Construct Assembly from C-Terminal Degradation Scheme

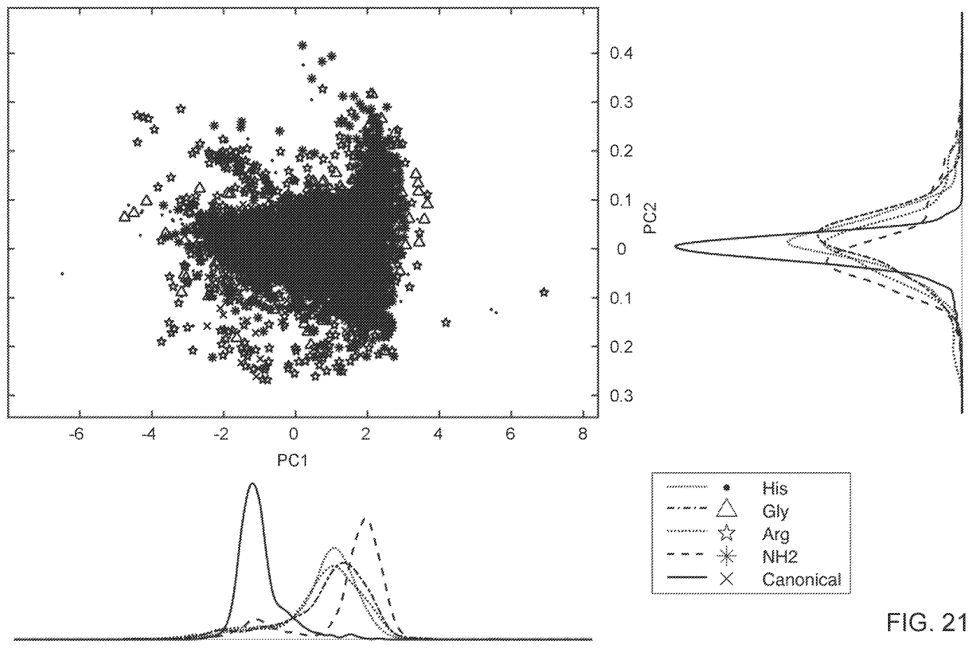

In the PROSE workflow, we describe the block-by-block assembly of the PROSE construct via (1) conjugation of an oligonucleotide to the N-terminal amino acid (NTAA) to a donor peptide (DP) of arbitrary sequence; (2) ligation of this NTAA-conjugated oligonucleotide to a seed strand (X), the preceding side chain-decorated block, or to an intervening monomeric or polymeric block that does not bear a side chain; (3) cleavage of the NTAA to liberate the AB-AA block; and (4) cyclic repetition of steps (2) and (3) until the desired number of amino acid side chains from the donor peptide are encoded within the PROSE construct in expanded form. However, there are analogous approaches whereby the PROSE construct can be assembled using methods of C-terminal degradation. That is, instead of sequentially encoding the “N”, “N-1”, “N-2”, . . . position amino acid side chains within the PROSE construct, the “C”, “C-1”, “C-2”, . . . position amino acids sequentially encoded within the PROSE construct (FIG. 21). As with the N-terminal degradation approach, the C-terminal degradation approach also encodes the identify of each subsequent amino acid within the polypeptide chain by retention of the respective side chain. Similarly, this C-terminal degradation approach also encodes within the PROSE construct the relative positioning of each amino acid from the DP with expanded spacing between consecutive side chains.

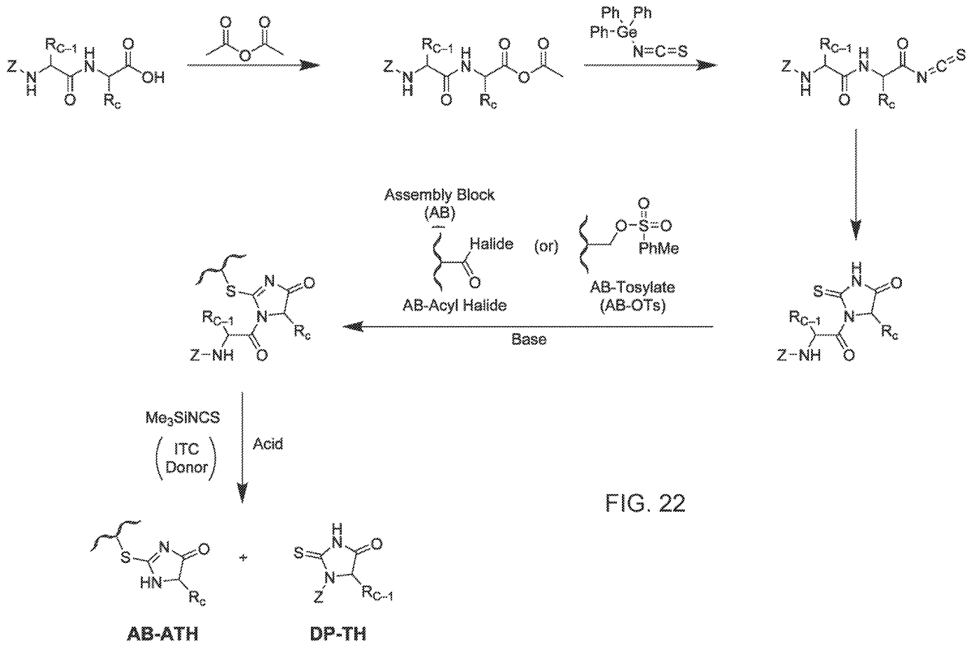

In one example of PROSE construct assembly via C-terminal degradation, poly-nucleotide assembly blocks (AB) bearing a leaving group, including but not limited to acyl halides and tosylates, are used to sequentially expand donor peptides of arbitrary sequence via C-terminal Schlack-Kumpf degradation (FIG. 22) (Li et al., Anal Biochem 302(1):108-13, 2002. doi.org/10.1006/abio.2001.5505). In this method, the C-terminal carboxylic acid is converted to an anhydride group by combining with acetic anhydride for 5 min at 50 to 80° C. before addition of 0.5 M triphenylgermanyl isothiocyanate (Ph₃Ge-ITC), or a similar, highly substituted analog, in acetonitrile. This solution is incubated at 50 to 80° C. for 30 to 60 min with mixing to form a peptide-thiohydantoin. The peptide-thiohydantoin solution is alkalized with reagents that can include triethylamine, sodium bicarbonate, and sodium borate and then combined with oligonucleotide modified with a leaving group, such as acyl chlorine or others. This results in conjugation to the oligonucleotide via the thiolate moiety (Boyd et al., Anal Biochem 206(2):344-52, 1992. doi.org/10.1016/0003-2697(92)90376-i). As with the N-terminal degradation workflow, of the C-terminal amino acid (CTAA)-conjugated AB is ligated to a seed strand (X), to the preceding side chain-decorated block, or to an intervening monomeric or polymeric block that does not bear a side chain. Subsequent addition of isothiocyanate anions, which can be generated from donors including (trimethylsilyl)isothiocyanate, under acidic conditions cleaves the CTAA, thus liberating the AB-alkylated thiohydantoin (AB-ATH) conjugate. This cleavage reaction reforms the peptide-thiohydantoin (DP-TH) at the C-terminus of the DP. After cleavage of the original “C” position amino acid CTAA from the peptide, the “C-1” position amino acid constitutes a newly-formed C-terminus and can then be referred to as the CTAA. As with the N-terminal degradation approach, this workflow can be repeated until the desired number of amino acids have been transferred from the DP and encoded, in expanded form, into the PROSE construct as SCDs in ATH form (FIG. 21).

The C-terminal degradation workflow has certain drawbacks, including stalling of degradation at proline residues owing to the increased substitution of its α-amino group (Boyd et al., Anal Biochem 206(2):344-52, 1992. doi.org/10.1016/0003-2697(92)90376-i). However, such drawbacks can be overcome via workflow adjustments, such as the strategic selection of the proteases used to generate the peptides. In this case, a person skilled in the art could select a protease or mixture of proteases that can selectively cleave the peptide bond N-terminal to prolyl residues, such that the component donor peptides generated from a protein will contain prolines at or near the N-terminus in positions “N”, “N-1”, “N-2”, or “N-3”. This would maximize the number of amino acids from each donor peptide that can be encoded within the PROSE construct prior to stalling at a C-terminal proline. Examples of such proteases can be found in references databases as described in “Enzymatic or Chemical Digestion”.

### (XII) Analogs of the Isothiocyanato Group

In all embodiments of the PROSE workflow, including those using N-terminal or C-terminal degradation approaches, isothiocyanate analogs can instead be used. These analogs include, but are not limited to, isoselenocyanates (ISCs) (Maeda et al., Heterocycles 82(2):2010. doi.org/10.3987/com-10-s(e)116; Iskierko et al. (in pol), Ann Univ Mariae Curie Sklodowska Med 3169-76, 1976). It can be attractive to use such analogs as they exhibit varying levels of reactivity and, thus, can enable design variations in the different reaction conditions used throughout the PROSE workflow. This facilitates the use of milder conditions, which can include shorter reaction times, lower temperatures, and less extreme pH. As described in “(V) Oligonucleotide Composition,” milder conditions can enhance the relative stability of the oligonucleotide or other polymeric assembly blocks (ABs) of the PROSE construct, which can improve fidelity of the fully-assembled PROSE construct and of the sequencing data produced from the construct.

### (XIII) Alternative Methods of Amino Acid Cleavage

In all embodiments of the PROSE workflow, including those using N-terminal or C-terminal approaches, enzymatic degradation can instead be used. In one example, enzymes, including proteases, peptidases, aminopeptidases, and Edmanases (US 2022/0227889A1), can be used to selectively remove either the NTAA or CTAA. This NTAA or CTAA can be pre-conjugated to an AB of the PROSE and this AB can be pre-ligated to the seed strand (X), the preceding AB, or an intervening monomeric or polymeric structure, such that enzymatic cleavage producing an amino acid side chain decorated block within the PROSE construct. As with the non-enzymatic approaches, this can be cycled repeatedly until the desired number of amino acids have been transferred from the DP and encoded, in expanded form and with retention of side chain identity and relative sequential positioning, into the PROSE construct.

### (XIV) PROSE Synthetic Long Reads and Linked Reads

As in NGS DNA sequencing, long reads are necessary to establish the true sequence of the protein, especially if there are repetitive regions. Once known, partial or short read sequencing is sufficient to identify the protein.

First approach is linked reads where Prose is employed for X number of cycles, where X could be 4 to 50 cycles. After X cycles, a number of amino acids are removed by Edman cleavage using PITC so it cannot ligate to the PSS or Prose construct, or aminopeptidases, or endoproteases, or chemical cleavage such as CNBr. After a specified amount of time or cleavage cycles, Prose can continue for an additional X number of cycles. A spacer block is added to delineate the continuous from the discontinuous reads This can be repeated N times, where N is at least 2. After sequencing, overlapping alignment of the fragments will be appreciated and can be used to produce a continuous sequence of the protein or peptide. For proteins at very low abundance, there may not be enough reads to derive a complete consensus sequence. In those cases, a second approach is used.

Synthetic long reads use multiple PSS strands that are encoded so that the order of PROSE sequences could be digitally reconstructed. Standard PROSE is performed for X number of cycles. A hairpin AB containing a UMI is ligated to the proximal AB on the prose strand. This hairpin has a UMI, cleavage motif that opens the hairpin. And a UMI complement that contains a reactive functionality that can react with the NTAA. After conjugation and then ligation, the hairpin is cleaved, and the PSS is cleaved from the docking linker. The first prose construct contains a barcode (from PSS) and a UMI (UMI-2). A new PSS strand is linked to the DL. This linkage is covalent so that the PSS-2 doesn't detach from the DL. This crosslinking can be T-dimer formation or others as described herein or known to those of skill in the art. PSS-2 can ligate to UMI-2′-AB to begin prose cycling again without an amino acid gap. After X prose cycles and after the final Edman cleavage, another UMI hairpin is conjugated to the NTAA, ligated to proximal end of Prose construct, hairpin opening, cleavage of PSS from DL. The 2^{nd} Prose construct contains the new UMI (UMI-2) and the previous UMI′. Multiple PROSE constructs can be aligned together because of the paired UMIs. Reconstruction would look like this:

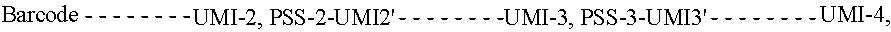

The long read method can help with overall efficiency because each prose construct's size could be controlled to maintain good kinetics and the PSS and AB blocks are exposed to less cycles of harsh chemicals. This method could also be combined with intermediate digestion steps as used in linked reads approach.

(XV) Barcode or UMI encoding.

In addition to standard AGCT-based barcodes, PROSE can use modified bases, such as methyl-C, or modified T, or alternate backbone such as PNA, morpholino, phosphothioate because nanopores are sensitive to more chemical changes than DNA sequencing.

### (XVI) Alternative Methods to Read the PROSE Construct

The PROSE construct is a versatile molecular framework that, in addition to nanopore-based sequencing, can be read and sequenced using other methodologies. For example, other single-molecule nucleic acid sequencing methods and spectroscopic methods can be used to ascertain both the identity and positional arrangement of amino acid side chain decorations (SCDs) on the PROSE construct at single-amino acid resolution. These methods are discussed herein.

### (XVII) Single Molecule Real Time (SMRT) Sequencing Technology

Pacific Biosciences instruments use a single molecule real time (SMRT) sequencing technology that monitors fluorescently-labeled nucleotides being added to the growing DNA strand that is complementary to the sequence of the analyte (Eid et al., Science 323(5910):133-8, 2009. doi.org/10.1126/science.1162986). Realtime monitoring captures the kinetics of the DNA polymerase incorporating nucleotides as well as processivity along the backbone of the analyte, which are both shown to be affected by the presence of modifications carried by the analyte DNA, such as methylation (Flusberg et al., Nat Methods 7(6):461-5, 2010. doi.org/10.1038/nmeth.1459). Therefore, the perturbation of kinetic parameters at or near the SCD of the PROSE construct is expected to be a signature of the amino acid, and hence can be used to sequence the donor peptide of the PROSE construct. Sidechain modifications could enhance perturbations of polymerase kinetics. Similarly, sidechain modifications may be designed to generate greater effects, such as interacting with the dye labeled nucleotides or with exposed domains of the polymerase enzyme. Additionally, SMRT sequences the forward and reverse strand because it is a circularized template that is read using the rolling circle method. If the SCD of the PROSE strand causes characteristic polymerase incorporation errors in the complementary strand during template and library generation, then this information can also be used to inform on the identity of the amino acid. Since the circular template is read 10-100 times, a consensus error profile could be obtained for additional confidence. Only a few amino acids need to be confidently identified to enable peptide alignment or mapping. With modifications to the SMRT technology, this could also be integrated with other spectral readouts such as fluorescence lifetime, which would be perturbed based on the local chemical environment of the amino acid side chain decorations (SCDs) on the PROSE construct.

### (XVIII) Sequencing by Synthesis

Illumina sequencing by synthesis (SBS) or Pacific Biosciences sequencing by binding (SBB) can be adapted to read PROSE constructs, which we refer to as PROtein Sequencing by Expansion and Amplification (“PROSE-A”). By barcoding the PROSE constructs with a suitable multimer of DNA and performing forward amplification, we can generate thousands of copies of the complementary stands. While this approach allows for amplification, we expect the SCD regions to introduce polymerase incorporation errors in the complementary strand, which can be quantified accurately by consensus sequencing of the thousands copies produced (Bentley et al., Nature 456(7218):53-9, 2008. doi.org/10.1038/nature07517). The error rate is expected to be a characteristic signature of the respective amino acid side chain decoration (SCD) borne by the PROSE construct. Therefore, this phenomenon could be used to identify the sequence of amino acid side chains encoded within the construct. Furthermore, this represents a novel method of indirectly amplifying the signal contributed by each amino acid contained within the donor peptide or protein, which could be particularly beneficial for amplifying the signal from low-abundance peptides or proteins or low-abundance modifications contained within, such as post-translational modifications (PTMs). Polymerases with different fidelities, natural or engineered, may produce different error fingerprints for different SCDs. Combinations of polymerases may result in higher accuracy amino acid calling and, thus, may expand the number of identifiable amino acids.

Another embodiment uses DNA sequencing for peptide identification. Preferably, certain SCDs on the PROSE construct are modified to include a DNA sequence region that encodes the identity of the amino acid and contains another region that is complementary to the PROSE strand. Various processes can be used to transcode the encoded SCD information into DNA sequence information which can then be sequenced using NGS technology.

Some amino acid sidechains, such as lysine, cysteine, must be blocked as they can form side reactions during ITC conjugation. Blocking chemistries can be designed to incorporate an orthogonal functional group that will withstand acidic conditions. A limited set of amino acid specific chemistries, for example 2 to 4, will be sufficient to enable peptide identification.

Aptamers that recognize the side chain specific blocking groups can also be used to both identify the amino acid and to provide primer sequences for primer extension or ligation as described. For bulkier sidechains, such as phenylalanine, an aptamer could bind directly to the residue (Cheung et al., ACS Sens 4(12):3308-3317, 2019. doi.org/10.1021/acssensors.9b01963). Since the sidechain groups are blocked or modified to mitigate side reactions, these groups could be selected to enhance or promote the binding of aptamers. The aptamer sequence itself could be used as the identifier of the cognate amino acid.

The modified SCD contains at least an amino acid DNA barcode sequence and a primer sequence complementary to the PROSE strand. The primer can hybridize and then be extended by a polymerase enzyme. If the polymerase is strand-displacing, then it will displace downstream primers and extended primers. The 3′ end of this complement strand will contain a UMI or barcode sequence and a reverse primer sequence that was incorporated into the assembly block (AB) or the seed strand (X). The reverse primer will generate a PROSE sense strand that incorporates the PROSE construct UMI/barcode identifier at the 3′ end and the amino acid barcode at the 5′ end. Only a single amino acid is encoded within each strand. The number of intervening Abs will be known by sequence analysis. The reverse complements are sequenced and aligned by UMI/barcode. Resulting overlapping alignments will identify the amino acid and relative position in the PROSE construct, such as xKxxxExxx+, where x is an AB containing the corresponding amino acid “x”, “K” is a lysine SCD, E is a glutamic acid SCD, and + is the UMI/barcode. This information can be used to map the sparse sequence information to protein databases.

If the polymerase is non-strand displacing and non-nick translating, then the primer will be extended until it reaches the 5′ end of another primer or the end of the PROSE construct.

In this embodiment, the primer design includes an upstream region from the barcode that can hybridize to the PROSE strand. The barcode sequence in the middle of this oligo is looped out. Sealing of the upstream nick by ligation, enzymatic or chemical, creates a reverse complement strand that contains multiple targeted SCDs. This RC strand is reverse primed to form a PROSE sense strand where SCDs have been replaced by amino acid barcode sequences.

Sequencing results can be aligned by UMI/barcode and will produce overlapping alignments that convey position and identity of a subset of amino acids. This information can be used to map the sparse sequence information to protein databases.

### (XIX) Spectroscopic Methods

The PROSE construct can also be read using other single-molecule techniques, including single-molecule spectroscopy. Intrinsic characteristics of fluorophores; such as fluorescence lifetime, fluorescence intensity, spectral positioning (and spectral shift); have been shown to be modulated by the local chemical environment surrounding the fluorophore (Jones Brunette et al., Biochemistry 53(40):6290-301, 2014. doi.org/10.1021/bi500493r). Therefore, perturbations to this local chemical environment, such as the fluorophore being positioned in close proximity to different chemical structures, can produce a characteristic modulation of one or more of these properties.

In one example, each assembly block (AB) of the PROSE construct consists of a unique poly-nucleotide sequence such that probes consisting of a poly-nucleotide sequence that is partially- or fully-complementary to a given AB and a fluorophore. These DNA-fluorophore probes are designed so that upon hybridization to the AB, the fluorophore is brought into immediate proximity of an amino acid side chain decoration (SCD) contained within that AB of the PROSE construct. Therefore, upon hybridization of the probe, the perturbation of the fluorescence lifetime (or other characteristic signals) of the fluorophore can be detected. In previous work, we have shown that the different fluorophores can exhibit different amounts of fluorescence lifetime perturbation according to the unique interactions between the fluorophore and the amino acid side chain, including weak, ionic, and aromatic attractions and/or interactions.

This workflow, called PROtein Sequencing by Expansion and Fluorescence Lifetime IMaging (“PROSE-FLIM”), allows for repeated interrogation of each amino acid side chain decoration (“SCD”) contained within the PROSE construct by probes bearing different fluorophores or fluorophores contained in different positions relative to the side chain.

Furthermore, the inclusion of synthetically modified nucleotides, including locked nucleic acids (LNAs), or backbone modification within the complementary region of the probe can alter the geometry of the formed duplex and thus can be used as an additional way to position the attached fluorophore relative to the SCD being interrogated. This is then repeated for each SCD contained within each AB of the PROSE construct to yield a matrix of fluorescence lifetime data. Since each set of probes will yield a characteristic fingerprint of fluorescence lifetime data according to the identity of the SCD, then using machine learning techniques the identity of each SCD can be determined. This PROSE-FLIM workflow is versatile in that it is agnostic to the identity of the SCD and, thus, can be used to detect canonical, non-canonical, synthetic, modified, or post-translationally modified amino acid side chains with no required alteration to the workflow.

### (XX) Other Applications of the PROSE Construct

It is contemplated that the PROSE constructs described herein will be useful outside of de novo peptide or protein sequencing. One salient feature of the PROSE construct is that it artificially expands the repertoire of chemical functional groups carried by an oligonucleotide polymer to any functional group present in canonical, post-translationally modified, non-canonical, unnatural, or synthetic amino acids. In one example, this expanded range of chemical functionality could make the PROSE construct useful in the development of more-effective aptamers. This would include aptamers containing ribonucleotides, deoxyribonucleotides, synthetic nucleotides, and backbone modifications. Using unmodified oligonucleotides, aptamers can only be selected to a limited set of targets and may have suboptimal binding characteristics inherent the limited chemical repertoire and highly negatively charged backbone of canonical oligonucleotides (Gold et al., PLOS ONE 5(12):e15004, 2010. doi.org/10.1371/journal.pone.0015004; Mayer et al., Nucleic Acid Aptamers: Selection, Characterization, and Application, 2nd ed. Springer US, 2022. ISBN 9781071626955). Therefore, the PROSE construct could expand the set of potential targets that aptamers can target and could also improve the binding properties of aptamers.

In one example, a candidate aptamer identified through selection can be modified with one or more site-specific side chain modifications by applying the PROSE workflow. In this example, the PROSE construct is designed to have the sequence of the candidate aptamer and SCDs can be designed at one or more specific sites within the construct to enhance the binding kinetics of the aptamer.

Combination of the PROSE construct with site-specific attachment to antibodies or creation of antibody hybrids may alter the specificity of therapeutics. Binding affinity is largely influenced by variable loop interactions with protein epitopes and the PROSE construct provides a hybrid approach to amino acid side chain interactions and DNA aptamer function which may result in reduced or augmented capabilities. The PROSE construct may also be used to create artificial epitopes of proteins that are difficult to produce recombinantly—further facilitating affinity based therapeutic development using display and directed evolution.

The PROSE construct could also be used as a random mutagenesis platform in vitro or in cellulo. The addition of an amino acid side chain may increase base incorporation defects or ligation defects when used in an enzymatic assay. Furthermore, certain amino acids may lead to patterned in-dels by enzymes.

### (XXI) Error Tracking and Monitoring

Step wise cycle efficiency is not 100% so monitoring for potential errors will help improve accuracy during aa calling or alignment.

PROSE consists of three steps: 1. Conjugation of AB block to NTAA; 2. Ligation of NTAA-AB to docking linker; 3. Cleavage of NTAA-AB-docking linker to form new NTAA. Failure can occur at any step, but ligation failures have the greatest consequence because they cause a deletion error where the amino acid is lost. If the position of this error is known, then gap alignment could be used to identify the missing amino acid if the peptide is already known. Other failures in conjugation or cleavage affect the sequence throughput for PROSE since those cycles don't produce a new NTAA. Possible failure modes include:
1. If conjugation fails, then ligation and cleavage steps will not occur. No sequence error introduced. The peptide will be lagging by one cycle.
2. If conjugation occurs, but ligation fails, and cleavage fails, then no sequence error introduced.
3. If conjugation occurs but ligation fails and cleavage occurs, then a deletion error is introduced. The NTAA-AB is lost. The cycle/position can be tracked if AB blocks are unique and alternating in each cycle then expect A-B-A-B-A-B. Where A-B-A-A-B would indicate cycle number and position in PROSE construct where this error occurred. In alignment process, gap alignment could be employed to identify missing amino acid.
4. If conjugation occurs and ligation occurs but cleavage fails, then no sequence error introduced.
5. If conjugation occurs and ligation occurs and cleavage occurs, then no sequence error introduced. This is an ideal cycle.

The above assumes adequate washes to remove reactants and no side reaction. In less than perfect conditions where unwanted side reactions occur.
6. If conjugation fails but ligation occurs and cleavage fails, then no sequence error. An AB insertion error will be detected by a non-SCD nanopore signal.
7. If conjugation fails and ligation fails and cleavage occurs (not necessarily at NTAA).

Sequence error introduced but difficult to determine breakpoint. This is a side reaction of all peptide sequencing using acidic conditions.

### (XXII) A Polypeptide Backbone for PROSE Construct

Expansion of peptides into PROSE construct can accomplished with other polymers besides DNA. DNA backbone advantageous because many DNA sequencers available. Non-DNA would be limited to nanopore or other direct readout techniques. Native polypeptide nanopore sequencing hampered by secondary and tertiary structure issues as well as other physicochemical properties such as hydrophobicity and variable mass-to-charge ratios.

Optimized peptide AB blocks can overcome these issues. The peptide AB blocks can be designed to have hydrophilic amino acids—natural, unnatural, synthetic—with a regular charge density, and which doesn't have any folding tendencies. Linking of peptide AB blocks can be accomplished by enzymatic methods, such as peptiligase (Toplak et al., Adv. Synth & Cataly. 358(13):2140-2147, 2016, doi.org/10.1002/adsc.201600017). If termini have DNA cohesive ends, then ligase enzyme could be used. Chemical approaches, including native chemical ligation (Agouridas et al., Chem Rev 119(12):7328-7443, 2019. doi.org/10.1021/acs.chemrev.8b00712), are often used to join peptides together. The reaction is fast and efficient but relies on cysteines which should be blocked after each conjugation reaction. Click chemistry can also be used where the click reactions at either end are orthogonal to prevent intra-molecular reactions. Peptide AB blocks with azide-peptideA-tet and tco-peptideB-alkyne can react to form linear constructs but not intramolecular circles. The peptideAB block for SCD could be azide-peptideA-tet and contain an ITC moiety for conjugation to NTAA. This could be ligated to the docking strand or the previous bridging block. The bridging block containing tco-peptide-alkyne is used to present tco on the growing PROSE construct.

An example of a growing PROSE strand: TCO-peptide-Alkyne: azide-peptide-TET (bridge): TCO-peptide-Alkyne: azide-docking strand

Step wise cycling as follows:
Conjugation of TCO-peptide-Alkyne to NTAA peptide
Chemical click of alkyne group to terminal azide group bridge (copper catalyzed)
cleavage [note that cleavage can also be after the bridge ligation]
Chemical click azide-peptide-TET (bridge) to terminal TCO on PROSE strand repeat

Bridging block adds an additional step to each cycle but also allows additional design or configuration options. For example, the TCO-peptide-Alkyne block can be shorter to facilitate conjugation kinetics or efficiency. The bridging block can be longer to provide desired SCD interval spacing but can also be designed to have specific charge density and structural parameters that help translocation through the nanopores. The peptide composition in either block could consist of natural & unnatural amino acids, peptide nucleic acids (PNA), synthetic spacer motifs (PEG, poly anions), modifications that are detectable by nanopores which can act as positional signposts.

In nanopores, detection sensitivity can be improved with slower transit time through the pore detection zone. Analyte interactions with the interior pore wall surface can affect residence time in or transit time through the pore. The PROSE construct could be modified with specific modifications that could slow down the translocation of the PROSE strand through the pore, leading to improved sensitivity. Modifications could be directly or indirectly attached to AB or bridging blocks.

### (XXIII) Exemplary Linker Structures

Examples showing the structure of a “C-modified T-C” trinucleotide representing an example of the repeat unit of Formula (1):

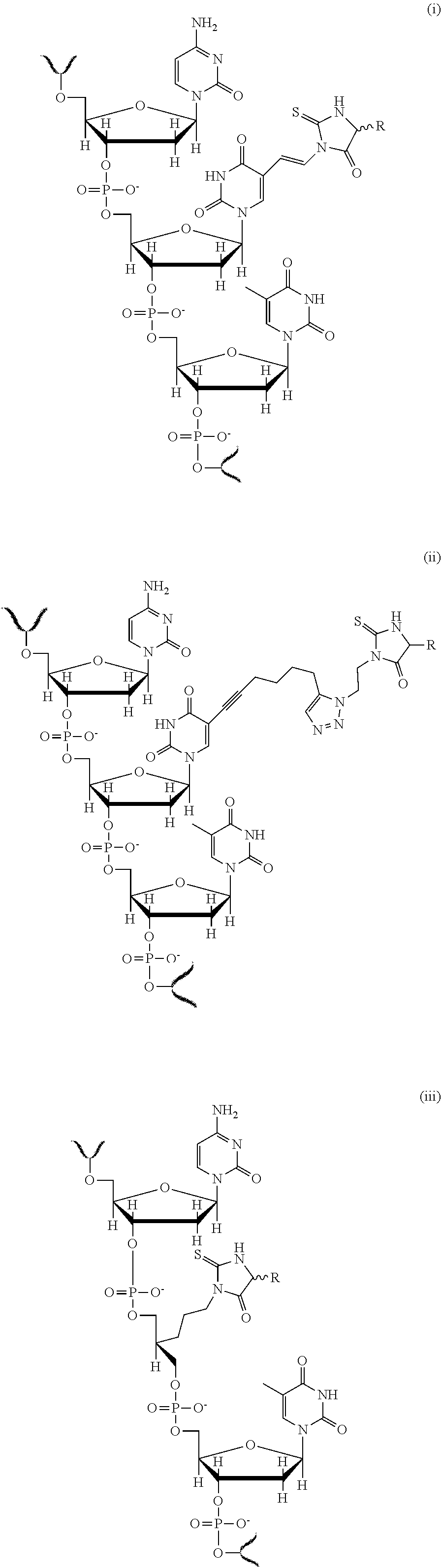

Examples of AB: purines—TTT(*)TTT—purines, where (*) is one of:
(i) a C6 amino linker dT (5-[2-[(6-aminohexyl)carbamoyl]ethenyl]-dU):

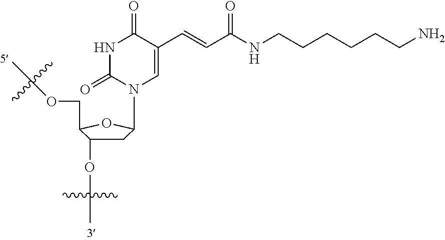 [SMILES: *OCC1OC(n2cc(/C=C/C(=O)NCCCCCCN)c(=O)[nH]c2=O)CC1O*]

(ii) a C2 amino linker dT: (5-[2-[(6-aminoethyl)carbamoyl]ethenyl]-dU):

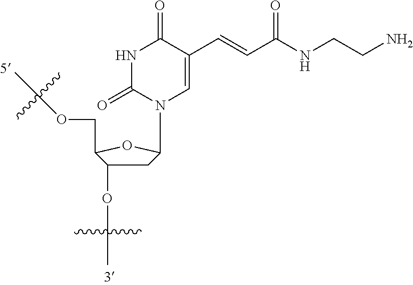 [SMILES: *OCC1OC(n2cc(/C=C/C(=O)NCCN)c(=O)[nH]c2=O)CC1O*]

(iii) a Uni-Link™ amino spacer::

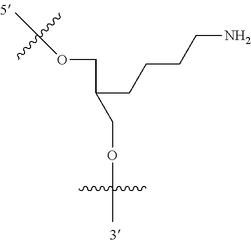 [SMILES: *OCC(CCCCN)CO*]

(iv) an alkyne modified dT (5-octadiynyl-dUan alkyne dU)

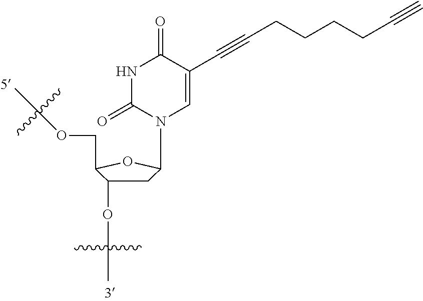 [SMILES: *OCC1OC(n2cc(C#CCCCCC#C)c(=O)[nH]c2=O)CC1O*]

(v) a C6 amino linker dC (5-[N-(6-aminohexyl)carbamoylmethyl]-dC):

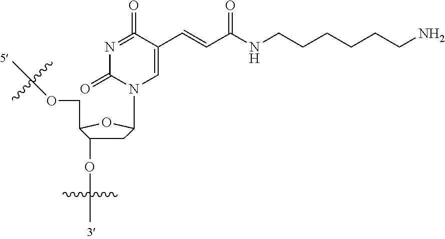

(vi) an alkyne spacer (1′-ethenyl-2′-deoxyribose):

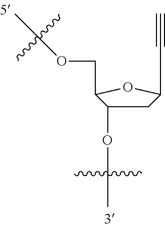 [SMILES: *OCC1OC(C#C)CC1O*]

### (XXIV) Synthesis of Isothiocyanate-Containing Oligonucleotides

To introduce an isocyanato functional group into an oligonucleotide, first a single stranded oligonucleotide containing at least one amino-modified nucleotide within the poly-nucleotide chain (can be carried as an internal modification of nucleotides within or at the ends of the oligonucleotide) is either synthesized or purchased commercially. Within the poly-nucleotide chain, the amino modification can be carried by one or more elements within a natural or synthetic sugar-phosphate backbone or by one or more nucleobases of the poly-nucleotide chain. Examples of nucleobases containing reactive functional group modifications include: 5-[2-[(6-aminohexyl)carbamoyl]ethenyl]-uracil, 5-[N-(6-aminohexyl)carbamoylmethyl]-uracil, 5-[2-[(6-aminopropyl)carbamoyl]ethenyl]-uracil, 5-[2-[(6-aminoethyl)carbamoyl]ethenyl]-uracil, 5-(3-amino-1-propenyl)-uracil, 5-[N-(6-aminohexyl)carbamoylmethyl]-cytosine, 5-octadiynyl-uracil, and 5-ethynyl-uracil (FIG. 6A). Examples of reactive group modified backbone include nucleosides with a 2′-amino-2′-deoxyribose, 2′-azido-2′-deoxyribose, or 1′-ethenyl-2′-deoxyribose; sugar spacers, such as Uni-Link™ amino spacer; terminators, such as 5′-azido-2′-deoxyribose, 5′-amino-2′-deoxyribose, 3′-azido-2′-deoxyribose, or 3′-amino-2′-deoxyribose; or peptide nucleic acids (PNA), such as abasic PNA or glycyl PNA (FIG. 6B). Fundamentally, any form of natural or synthetic nucleotide or nucleotide analogue including a reactive amino group can be used. This includes amino modifications to a natural, synthetic, or unnatural nucleic acid backbone (peptide nucleic acids (PNA), locked nucleic acids (LNA), alkyl phosphoramidites, phosphorothioates, etc.) and amino modifications to nucleobases or analogues. Additionally, any ssDNA sequence containing an amino modification can be used. The natural primary amine of nucleobases adenine, cytosine, and guanine can potentially be converted to the isocyanato group using this reaction scheme, but differential reactivity of the primary amines can be leveraged to reduce these undesirable side products. Furthermore, natural purine nucleotides have lower thermal and pH stability than natural pyrimidine nucleotides, which can result in depurination and potential scission of the backbone. Therefore, downstream processes of the workflow may guide design of the oligonucleotide. Inclusion of nucleotides with synthetic modifications to the backbone elements and/or to the nucleobases can increase the chemical stability of poly-nucleotides beyond that of canonical poly-nucleotides. Additionally, click chemical reactions, such as reactions involving azide, DBCO, alkynes, TCO, tetrazine, and others, can be used to directly add an ITC moiety via small molecule conjugation (e.g., reaction with a small molecule containing the complementary click reagent and an ITC).

In one example, a single stranded DNA (ssDNA) oligonucleotide containing either one 5-[2-[(6-aminohexyl)carbamoyl]ethenyl]-uracil or one 5-[2-[(6-aminoethyl)carbamoyl]ethenyl]-uracil amino-modified nucleobase in the poly-nucleotide chain is used. The amino-modified ssDNA oligonucleotide assembly block (“AB-NH₂”) is dissolved in 1:2 v/v ultrapure water in dimethyl sulfoxide (DMSO), mixed via pipette. For the synthetic step, to this AB-NH₂ solution, 0.6 molar equivalents of triethylamine, 0.03 molar equivalents of 4-dimethylaminopyridine, and 1000 molar equivalents of carbon disulfide (5 M in tetrahydrofuran) are added. The final pH of the solution is typically pH 8 to 9. This was mixed at room temperature with for 15 min to 2 h. The reaction flask is placed in a 0° C. ice bath and 1.1 molar equivalents of di-tert-butyl dicarbonate or tosyl chloride are added. After reacting for 2 to 12 hours, the mixture is then lyophilized at approximately −105° C. until completion. The synthetic and lyophilization steps can be cycled several times, if needed, until a sufficiently high-purity isothiocyanato-modified oligonucleotide assembly block is obtained (AB-ITC), as determined by various analytical methods (liquid chromatography-mass spectrometry (LC-MS), gel electrophoresis, UV-vis spectroscopy, Fourier transform-infrared spectroscopy (FTIR), nuclear magnetic resonance spectroscopy (NMR), etc.). Typically, 1 to 5 cycles are performed.

In another scheme, the amino-modified ssDNA oligonucleotide assembly block (“AB-NH₂”) is dissolved in 1:2 v/v ultrapure water in dimethyl sulfoxide (DMSO), mixed via pipette to achieve a 100 μM concentration of DNA. 3% volume of pure triethylamine is added to the solution, bringing the pH to around 9. By means of a needle, 11.5% volume of pure CS₂ (carbon disulfide) prechilled to 4° C. is added to the bottom of the flask, where it forms a layer separated from the rest of the mix. The vial cap is tightly sealed with a Teflon-lined lid. The solution reacts at 4° C. for 48 hours on an orbital vortex mixer (vortexer) in vibrational mode. The reaction is monitored for a color change in the upper aqueous phase, which will turn yellow upon successful modification. Once the 48 hours have passed, the solution is mixed with a pipette and the layers temporarily combined in a single phase. CS₂ and Et₃N (triethylamine) are removed by rotary evaporator at 50° C. The product is then purified via HPLC and lyophilized at approximately −105° C. High-purity isothiocyanato-modified oligonucleotide assembly block (AB-ITC) is obtained, as determined by various analytical methods (liquid chromatography-mass spectrometry (LC-MS), gel electrophoresis, UV-vis spectroscopy, Fourier transform-infrared spectroscopy (FTIR), nuclear magnetic resonance spectroscopy (NMR), etc.). An example reaction: 50 μL 1 mM P40—NH₂, 450 μL Optima LCMS water, 1 mL DMSO, 45 μL Et₃N, 173 μL CS₂.

In an alternative scheme, the isothiocyanato group is introduced into the oligonucleotide via a click reaction. In one example of this, an oligonucleotide containing a primary amine modification, including those shown in FIG. 6, can be reacted with a bifunctional linker molecule containing N-succinimidyl (NHS) ester and DBCO moieties (NHS-DBCO) to introduce a DBCO group. Amine-reactive groups other than NHS ester can also be used. After reaction completion, the remaining free NHS-DBCO in solution is removed via liquid chromatography, desalting, membrane filters, and/or single-use columns. The DBCO-modified oligonucleotide is reacted with a bifunctional azide-isothiocyanate molecule for several hours in aqueous or anhydrous solvents that do not contain any primary amines, thiols, or other isothiocyanato-reactive functional groups. The resulting oligonucleotide is purified via commercially available membrane filters or single-use columns.

In another alternative scheme, the isothiocyanato group of a bifunctional linker molecule is first attached to the N-terminus of a peptide and then a modified oligonucleotide is reacted to the second linker moiety to conjugate the peptide and oligonucleotide. For example, the isothiocyanato group of a bifunctional azide-isothiocyanate molecule is conjugated to the N-terminal amino acid (NTAA) of the peptide. Next, an oligonucleotide containing a primary amine modification, including those shown in FIG. 6, can be reacted with a bifunctional linker molecule containing N-succinimidyl (NHS) ester and DBCO moieties (NHS-DBCO) to introduce a DBCO group. Amine-reactive groups other than NHS ester can also be used. After reaction completion, the remaining free NHS-DBCO in solution is removed via liquid chromatography, desalting, membrane filters, and/or single-use columns. This DBCO-modified oligonucleotide can then be efficiently reacted with the azido group attached to the N-terminus of the peptide. This approach offers advantages including improved reaction efficiency, long-term stability of the modified oligonucleotide, and less reagent waste.

In an alternative embodiment, a molecule or set of molecules including an oligonucleotide, nucleoside, or abasic nucleoside of canonical, non-canonical, partially-synthetic, or fully synthetic structure is further modified for non-enzymatic, for example chemical, ligation reactions. These molecules can be designed to bear two natural or synthetic modifications that facilitate selective conversion to a linker molecule, such as a click reagent. In one example, these modifications can be borne by the 5′ and 3′ ends of the molecule (or, alternatively, the 5′ and 2′ ends) and can be designed with or converted to linker groups, such as the following click groups: dibenzocyclooctyne (DBCO), azide, trans-cyclooctene (TCO), and/or tetrazine (FIG. 3).

In another embodiment, oligonucleotide or nucleoside that is trifunctional modified to the 3′ and 5′ carbons and nucleobase can be used as analog for PROSE construct. 3′ and 5′ carbons are functionalized with click linker such as DBCO, azide, TCO, tetrazine. Nucleobase is functionalized with primary amine which later is converted to ITC. In this embodiment (scheme) the PROSE construct is built without enzymatic reaction (no ligase), but by chemical reaction that can be done in either 100% organic solvent or combination of organic and aqueous solvent (water). In this embodiment, there are at least two interchangeable analogs. At least one or more analogs can have functionalized ITC. The analog that does not have ITC only has two chemical linkers on 3′ and 5′ carbon, and act as a spacer. The spacer is used to add space between AAs, the spacer is used as sequencing backbone during nanopore sequencing.

One or more nucleobases, sugars, backbone sites, or abasic sites within these molecules can be functionalized with a reactive group or protected reactive group, such as a primary amine, tert-butyloxycarbonyl (Boc)-protected amine, or fluorenylmethyloxycarbonyl (Fmoc)-protected amine, which can subsequently be converted to an isothiocyanato group or to a click linker for subsequent attachment to an isothiocyanato-bearing linker or to a peptide-isothiocyanato conjugate.

### (XXV) Synthesis of Multi-Functional Docking Linkers (DLs)

As discussed in the Section entitled “(X) Docking Linker (DL) Design,” in some embodiments of the PROSE workflow, a multi-functional docking linker (DL) is used to attach molecules of interest, such as a peptide and a seed strand (X) for the PROSE construct, to, in some embodiments, a solid or semi-support. Examples of these DLs are shown in FIG. 9.

In one example, a tri-functional docking linker (DL) is produced via an N-alkylation reaction of a preformed secondary amide bearing methyltetrazine and dibenzocyclooctyne (DBCO) groups:
1. An aliquot of methyltetrazine-PEG24-DBCO is dissolved in acetonitrile.
2. To the solution, 1 molar equivalent of Bromo-PEG3-phthalimide is added to the solution.
3. To the reaction mixture, 2 molar equivalents of potassium phosphate and 2 molar equivalents of tetrabutylammonium bromide are added.
4. The reaction is stirred at 250 rpm and 50° C. for 48 h under reflux. Acetonitrile is occasionally added to replenish any volume lost to evaporation.
5. The solution is filtered with a 0.22 μm syringe filter to remove insoluble matter.
6. The product is confirmed by liquid chromatography-mass spectrometry (LC-MS) and purified via reverse phase high-performance liquid chromatography (RP-HPLC).
7. To the HPLC-purified fraction, 10 molar equivalents of hydrazine are added. This solution is stirred for 4 h at room temperature.
8. The product is confirmed by mass spectrometry and purified via RP-HPLC.
9. The final product is lyophilized overnight and stored at −20° C. The structure is confirmed via ¹H and ¹³C NMR, liquid chromatography-mass spectrometry (LC-MS), and Fourier-transform infrared spectroscopy (FTIR).

To test the functionality of this tri-functional docking linker (DL), freshly synthesized DL was attached to the surface of glass beads via the one of the three reactive group. The two remaining functional groups were conjugated to fluorophores bearing complimentary reactive groups and then washed. The complete assembly was deposited on a glass coverslip in imaging buffer and investigated using total internal reflection fluorescence microscopy (TIRFM).

Colocalized emission from NHS-AF547 and TCO-AF488 confirmed the presence of the newly formed primary amine and pre-existing methyltetrazine, respectively.

In a second example, a tri-functional docking linker (DL) is produced via an N-alkylation reaction of a preformed secondary amide bearing trimethoxysilane and dibenzocyclooctyne (DBCO) groups:
1. An aliquot of DBCO-trimethoxysilane is dissolved in acetonitrile.
2. To the solution, 1 molar equivalent of bromo-PEG3-phthalimide is added.
3. To the reaction mixture, 2 molar equivalents of potassium phosphate and 2 molar equivalents of tetrabutylammonium bromide are added to the reaction mixture.
4. The reaction is stirred at 250 rpm and 50° C. for 48 h under reflux. Acetonitrile is occasionally added to replenish any volume lost to evaporation.
5. The solution is filtered with a 0.22 μm syringe filter to remove insoluble matter.
6. The product is confirmed by liquid chromatography-mass spectrometry (LC-MS) and purified via reverse phase high-performance liquid chromatography (HPLC)
7. To the HPLC-purified fraction, 10 molar equivalents of hydrazine are added. This solution is stirred for 4 h at room temperature.
8. The product is confirmed by mass spectrometry and purified via RP-HPLC.
9. The final product is lyophilized overnight and stored at −20° C. The structure is confirmed via ¹H and ¹³C NMR, liquid chromatography-mass spectrometry (LC-MS), and Fourier-transform infrared spectroscopy (FTIR).

In a third example, a tri-functional docking linker is produced from a protected amino acid starting material.
1. An aliquot of a lysine monomer with a tert-butyloxycarbonyl (Boc) protecting group on the amino side chain (NE-Boc-L-lysine CAS: 2418-95-3) is dissolved in a mixture of 5% dimethyl sulfoxide v/v in dimethylformamide. The pH of the solution is adjusted to 8.5 by addition of triethylamine.
2. To this solution, 1.5 molar equivalents of a bifunctional linker, such as dibenzocyclooctyne-hexyl-N-succinimidyl (NHS) ester (DBCO-C6—NHS, CAS: 1384870-47-6), is added to the solution and reacted for 2 h. Whereas NE-Boc-L-lysine is not fully soluble in this reaction mixture, the final product is, so the reaction progress is tracked by the gradual clarification of the mixture.
3. The reaction is complete when a clear solution is obtained. The product is lyophilized and then isolated using a CombiFlash flash chromatography purification system.
4. The product is resuspended in 0.01% v/v triethylamine in dimethylformamide and 1.1 molar equivalents of a carboxyl activating agent, such as N,N′-dicyclohexylcarbodiimide (DCC, CAS: 538-75-0), and 1.1 molar equivalents of (3-aminopropyl)triethoxysilane or (3-aminopropyl)trimethoxysilane are added.
5. The reaction is stirred overnight at room temperature and the final product is isolation using a CombiFlash flash chromatography purification system.

In one example of the workflow, this product is conjugated activated solid support of activated glass or silicon. Unreacted material is removed with multiple methanol or ethanol washes. The Boc protecting group is removed from the lysine side by washing with 1% v/v aqueous TFA followed by 15 to 60 min incubation in the same at room temperature. The solid support with the bound tri-functional docking linker (DL) is then cleaned with multiple methanol or ethanol washes.

### (XXVI) Conjugation of Peptide to the Oligonucleotide Seed Strand (X)

The conjugation of a peptide or protein to the oligonucleotide seed strand (X) can be performed either in solution or on a solid or semi-solid support. Use of a solid or semi-solid support allows for more-convenient removal of unreacted components and for exchange of solvent or buffer conditions. Furthermore, a solid or semi-solid support can aid in maintaining spatial separation between peptide-X conjugates to minimize potential intermolecular reactions between different conjugates in the proceeding steps of the workflow. Solid or semi-solid supports can include surfaces, such as glass, plastic, ceramic, and/or metal; particles, such as nanometer-, micrometer-, or millimeter-sized particles composed of materials including polystyrene, iron oxide, tentagel, glass, ceramics, and/or plastic; and other shapes and forms of matter.

In one example, an internally-biotinylated DNA seed strand (X) with a 5′ dibenzocyclooctyne (DBCO) group is incubated with magnetic beads bearing covalently-bound streptavidin in phosphate buffered saline supplemented with 0.01% Tween 20 (PBS-T). The reaction proceeds at room temperature on a shaker for 5 to 15 min. The mixture is washed on a magnetic rack and resuspended in PBS-T. A 1 Ox molar excess of peptide containing a C-terminal azido modification is added to the streptavidin-bead solution and incubated at room temperature for at least 30 min to yield peptide-X-bead conjugates. After 1 to 3 centrifugal washes in 1× PBS-T to remove excess reagents, the peptide-X-bead conjugates can be used directly or stored at 4° C.

Surface Attachment of Peptide-Oligonucleotide Seed Strand Conjugate
POC approach with SA-iron oxide particles, peptide-azide, modified DNA
Bifunctional/Trifunctional
Surface attachment such as: surface, bead, gel, or other material
Vs solution approach
Typical attachment protocols and variations

### (XXVII) Conjugation of Oligonucleotide-Isothiocyanate (AB-ITC) to N-Terminal Amino Acid (NTAA)

An isothiocyanto-modified assembly block (“AB-ITC”) is reacted with the primary amine of the N-terminus of the peptide or peptide-oligonucleotide seed strand conjugate in basic conditions, such as pH 8 to 9.5. In some examples, this conjugation reaction is performed in an organic solvent (including methanol, ethane-1,2-diol, 1,4-dioxane, dimethylformamide, acetonitrile, dimethyl sulfoxide, ethanol, etc.), in water, in standard biological buffers, with salts present (including NaCl and/or MgCl₂), with an alkylated quaternary amine compound, with a reducing agent present (dimethylphenylsilane, tris(2-carboxyethyl)phosphine (TCEP), etc.), or in various combinations of these conditions. Salts or alkylated quaternary amines can help solubilize oligonucleotides under various conditions. Reducing agents can prevent or reduce the formation of undesirable side products. Anhydrous organic solvents or mixtures of these solvents with water can prevent or reduce the formation of undesirable side products.

The reaction is alkalized with reagents that can include triethylamine, sodium bicarbonate, and sodium borate. The requirement for an alkalizing reagent is that it must exhibit low or absent nucleophilicity toward the isocyanato group. This precludes the use of chemicals containing reactive functional groups including primary or secondary amines and thiols. The reaction is performed at room temperature or with heating, including but not limited to temperatures in the range of 40 to 70° C., for 15 min to 3 h.

In one example, the reaction is performed in 1× phosphate-buffered saline (PBS) solution supplemented with 0.2% v/v triethylamine (final pH 8 to 9) using an approximate 2:1 molar ratio AB-ITC to peptide-oligonucleotide seed strand conjugate (peptide-X).

The reaction is typically incubated with shaking at 40 to 60° C. for 30 min to 1 h to yield the AB-TU-peptide-X conjugate, for which the AB-ITC and peptide-X are linked by a thiourea (TU) functional group. Higher molar ratios of AB-ITC to peptide-X can be used to expedite the conjugation and to potentially increase the yield of AB-TU-peptide-X. Similarly, lower molar ratios of AB-ITC to peptide-X can be used to reduce reagent waste, but this can be at the cost of higher reaction times and reduced AB-TU-peptide-X yield.

This reaction can be performed either in solution or on a solid support. Use of a solid support allows for more-convenient removal of unreacted components and for exchange of solvent or buffer conditions. Furthermore, a solid support can aid in maintaining spatial separation between AB-TU-peptide-X conjugates to minimize potential intermolecular reactions between different conjugates in the proceeding steps of the workflow. Solid supports can include surfaces, such as glass, plastic, ceramic, and/or metal; particles, such as nanometer-, micrometer-, or millimeter-sized particles composed of materials including polystyrene, iron oxide, tentagel, glass, ceramics, and/or plastic; and other shapes and forms of matter.

In one example, the ITC group of a bifunctional linker is first reacted with the N-terminus of the donor peptide or protein (DP) and the second reactive group of the bifunctional linker is subsequently conjugated to a modification borne by the AB. As described, this second reactive group and the AB modification can include chemical groups that undergo highly efficient click reactions.

In another example, AB-ITC can be conjugated to the N-terminus of surface-immobilized peptides using pyridine and DiPEA as solvents. By way of illustrative example, immobilized peptides are washed 3× with 10% v/v pyridine in nuclease free water and subsequently washed 2× with neat pyridine to remove residual water and condition the substrate. A 10× molar excess over peptide of AB-ITC in pure molecular grade water is premixed into a solution consisting of 22:1 v:v pure pyridine and DiPEA, respectively, and 25 mM Strontium chloride. The reaction proceeds for 90 minutes with shaking at 50° C. before being washed with 10% v/v aqueous pyridine. The final PROSE construct is generated as described, and sequenced on the Oxford Nanopore platform. This conjugation method yielded 4.19 fmol of total PROSE product compared to 0.36 fmol for the negative control.

### (XXVIII) Ligation of Assembly Block-Isothiocyanate-Peptide (AB-ITC-Peptide) Conjugate to Oligonucleotide Seed Strand (X) or to the Preceding AB or Intervening Monomeric or Polymeric Sequence

AB-ITC attached to the peptide by the primary amine of the NTAA (the AB-TU-peptide-X conjugate) is ligated to the seed strand (X) (for the first cycle of the workflow) or to the preceding AB or intervening monomeric or polymeric sequence (IS, all subsequent cycles) using a proximity ligation approach. A single splinting hybrid strand (can alternatively be multiple reverse complement strands) with partial homology to both the seed stand (X) and AB-ITC strands is annealed in an appropriate buffer (Neb 2.0 or other buffers including MgCl₂ and/or NaCl) by heating to 60 to 90° C. (depending on secondary structure, melting temperature (Tm), and other factor) followed by stepwise cooling to 4° C. In one example, cooling is performed at a controlled rate of 2° C. per second. The buffer is exchanged to an appropriate buffer for ligation, such as Neb Quick Ligase Buffer or comparable buffer, and ligated at room temperature for 5 to 30 minutes by either chemical (Kollaschinski et al., Bioconjug Chem 31(3):507-512, 2020. doi.org/10.1021/acs.bioconjchem.9b00805; Fantoni et al., Chem Rev 121(12):7122-7154, 2021. doi.org/10.1021/acs.chemrev.0c00928) or enzymatic ligation. In one example, the bacteriophage-derived T4 DNA ligase is used.

In other examples, chemical ligation is performed via standard click reactions including, but not limited to, the following click linker groups: dibenzocyclooctyne (DBCO), azide, trans-cyclooctene (TCO), and/or tetrazine.

Upon ligation of the AB-TU-Peptide-X to X (for the first cycle of the workflow) or to X-AB-SCD, X-(AB-SCD)-IS, X-poly(AB-SCD), or X-poly((AB-SCD)-IS) (for subsequent cycles), an intermediate cyclical construct (ICC) is formed.

### (XXIX) Edman Degradation to Remove N-Terminal Amino Acid (NTAA) and Liberate the Assembly Block-Side Chain Decoration (AB-SCD)

Edman degradation is performed to remove the N-terminal amino acid (NTAA) and to expose the N-terminal amine of the next amino acid in the polypeptide chain (the “N-1” position amino acid. Upon cleavage of the NTAA, the AB-SCD is liberated, which opens the intermediate cyclical construct (ICC), and completes the addition of an AB-SCD block to the PROSE construct that is being assembled.

In one example, aqueous trifluoroacetic acid at a concentration of 0.5 to 1% v/v (pH 1 to 2) is added to the ICC and incubated for 30 to 60 min at 40 to 70° C. In other examples, different Bronsted-Lowry acids, including acetic acid, trichloroacetic acid, phosphoric acid, phosphorous acid, methanesulfonic acid, hydrochloric acid, hydrofluoric acid, or hydroiodic acid or Lewis acids, including scandium trifluoromethanesulfonate, boron trifluoride (neat, etherate, tetrahydrofuran, or acetic acid), silver trifluoromethanesulfonate, or yttrium trifluoromethanesulfonate can be used in anhydrous solvents, including dimethylformamide, acetonitrile, dimethyl sulfoxide, 1,2-ethanediol, methanol, ethanol, tetrahydrofuran, or 1,4-dioxane, aqueous solvents, or water with or without reducing agents, including dimethylphenylsilane, tris(2-carboxyethyl)phosphine (TCEP), or phosphorous acid at temperatures ranging from 20 to 80° C. for 10 to 120 min. Furthermore, use of isothiocyanate analogs, especially the more reactive isoselenocyanate (ISC) further expands the set of possible reaction conditions as the reactions can be performed under milder chemical conditions, at lower temperatures, and in less time.

While it can be favorable to have all PROSE construct side chain decorations (SCDs) in a single form, either the thiocarbamate (TC) or thiohydantoin (TH) form, it is not strictly necessary as each form will have a characteristic readout when it passes through the nanopore. However, the disadvantage of a TC and TH mixture is that it doubles the set of possible SCDs. For example, the 21 canonical amino acid side chains would have a set of 42 TC and TH forms.

As discussed in “(XIII) Alternative Methods of Amino Acid Cleavage,” a C-terminal chemical degradation approach or a C- or N-terminal enzymatic degradation approach can be used as an alternative to this N-terminal chemical degradation approach.

(XXX) Cycling of this Process to Produce the Amino Acid-Decorated PROSE Construct

The conjugation, ligation, and degradation steps described in “(XXVII) Conjugation of Oligonucleotide-Isothiocyanate (AB-ITC) to N-Terminal Amino Acid (NTAA),” “(XXVIII) Ligation of Assembly Block-Isothiocyanate-Peptide (AB-ITC-Peptide) Conjugate to Oligonucleotide Seed Strand,” and “(XXIX) Edman Degradation to Remove N-Terminal Amino Acid (NTAA) and Liberate the Assembly Block-Side Chain Decoration (AB-SCD)” are then repeated until all or a desired number of amino acid side chain have been transferred from the donor peptide or protein (DP) and encoded into the PROSE construct as side chain decorations (SCDs) in expanded form.

### (XXXI) Workup of a Representative PROSE Construct

The single-stranded PROSE construct can either be converted into a double-stranded form or used in its single-stranded form, depending on the intended application. Second strand synthesis of the oligonucleotide expanded peptide (the single-stranded PROSE construct) is typically performed using a polymerase that is processive over large nucleotide modifications.

In one example, a hybrid strand for the 3′ end of a side chain decorated, support-bound PROSE construct is annealed and extended by the exonuclease null small Klenow fragment of DNA Polymerase I in NEB 2.0 buffer with 10 μM of each canonical deoxynucleotide triphosphate (dNTP) for 15 to 60 minutes at 37° C. The resulting support-bound duplex side chain decorated hybrid (the double-stranded PROSE construct) is then washed and immersed in ultrapure molecular grade water. The double-stranded PROSE construct is then liberated from the support by exposure to ultraviolet light for 15 to 60 minutes at room temperature. Duplex DNA hybrids can be formed by many other polymerases, including phi29, DNA Pol I, T4, T7, Bst 2.0, phusion, and kappa.

In a second example, the support-bound oligonucleotide expanded peptide is directly liberated by exposure to ultraviolet light for 15 to 60 minutes at room temperature to yield the single-stranded form of the PROSE construct.

In a third example, the oligonucleotide expanded peptide is produced without attachment to a solid support. This single-stranded form of the PROSE construct can be converted to the double-stranded form as described in the first example.

In a fourth example, the oligonucleotide expanded peptide is produced without attachment to a solid support. This single-stranded form of the PROSE construct can be used directly.

### (XXXII) Preparation for Nanopore Sequencing

The PROSE construct is compatible for use with a broad variety of nanopore-based sequencing technologies, both existing and in development. Based on the requirements of the nanopore technology used, the PROSE construct can be prepared in either single-stranded or double-stranded forms and then directly used in technology-specific protocols and workflows.

These protocols and workflows often require the addition of structural elements to inputted poly-nucleic acid material including, but not limited to, adapters, primers, supports, and/or modifications. The PROSE construct is seamlessly compatible with these requirements.

In one example, the double-stranded PROSE construct is prepared for sequencing on either MinION, GridlON, or PromethlON devices using the standard, manufacturer-provided protocols for the proprietary ligation sequencing kit LSK-110 or LSK-114 for use with proprietary flow cells MIN106D or MIN114, respectively. The PROSE construct has been demonstrated to be fully compatible with these protocols. Furthermore, the PROSE construct has been successfully sequenced on the MinION, GridlON, and PromethlON devices to yield data corresponding to the nucleic acid sequence and to each amino acid side chain encoded within the construct.

### (XXXIII) Software, Data Processing, and Data Analysis

The sequencing runs of the PROSE constructs on MinION, GridlON, and PromethlON nanopore sequencers generated raw data containing current versus time data (.FAST5 or pod5 file format) and sequence data (.FASTQ file format). These data were further analyzed using custom python scripts.

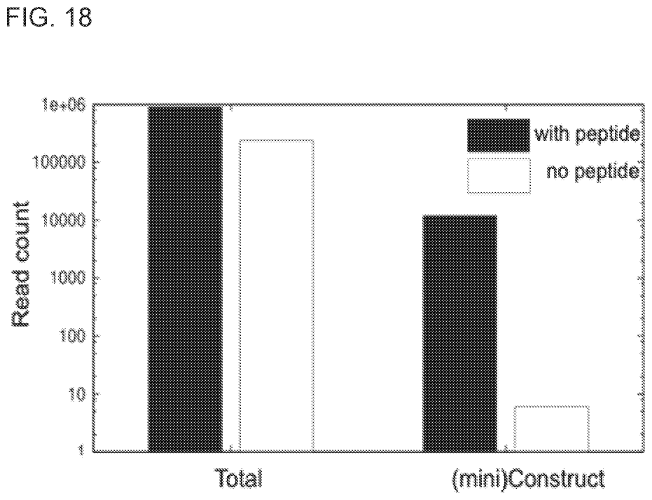

Each sequencing run of the PROSE Constructs, which were generated by the procedure described in Examples 2 and 8, generated millions of reads on the nanopore sequencer and were either classified as pass or fail based on the quality of the reads. The FASTQ files were first demultiplexed using standard NGS procedures. Then, demultiplexed reads were aligned against specific non-variable or non-modified regions of the construct in the specific order used to create them. Results were summarized as percentage of number of correct reads over total reads produced for a specific condition. FIG. 18 shows the number of aligned reads of the Construct are significantly enhanced in the experiment with the peptide compared to the mock experiment (without peptide), highlighting the Constructs indeed carry the amino acid chain decorations. As the DNA with the SCD impacts the signal when it travels through the pore, those perturbations appear as insertions often base called as a random string containing purines in an otherwise pyrimidine only region of the sequence as shown in FIG. 19.

To extract the raw signal associated with the modified groups, we extracted the FAST5 files containing the filtered reads. The FAST5 files were processed with Tombo or other open access software for re-squiggling and assignment of the bases to specific regions of the raw signal, which allows extraction of the signal associated with specific bases for further analysis.

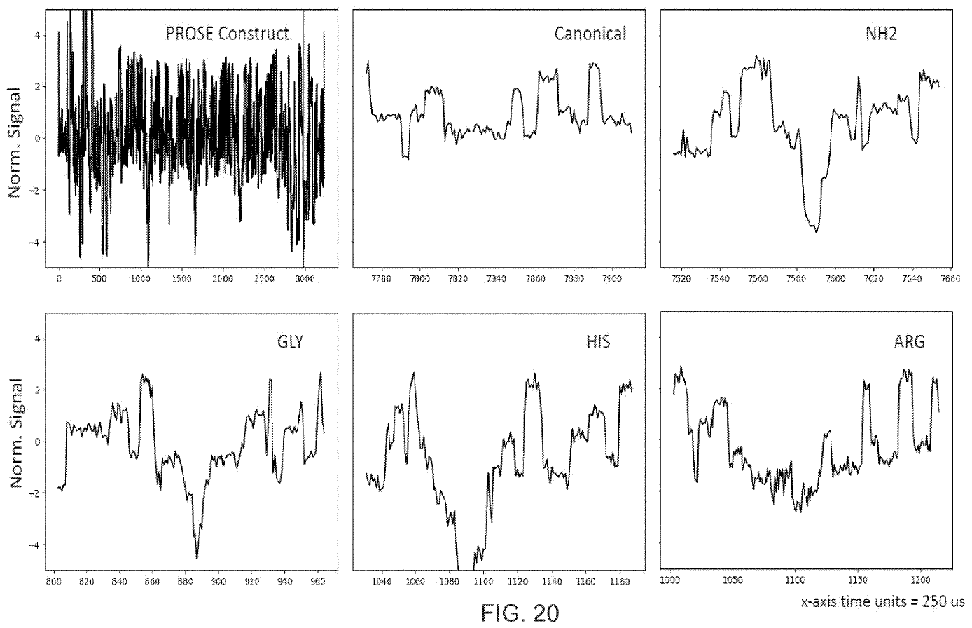

For the runs performed according to Example 9, the raw signal thus extracted for the whole Construct and specific regions of interest, such as TTT-X-TTT, where X corresponds to the SCD, are shown in FIG. 20. We further analyzed the raw signal for characterization the changes associated with different modifications (SCD) of the DNA. Specifically, we quantified the mean and variance of the mean absolute deviation (MAD) normalized signal, and performed principal component analysis (PCA) for clustering purposes (see FIG. 21). Each read was clustered with the amino acid known with positional accuracy and fit to a cumulative distribution to determine the Kolmogorov-Smirnov statistical interpretation of the maximum distance between curves (D-max) (FIG. 20). P-values for each pair were calculated using the Peacock method (Peacock, Monthly Notices of the Royal Astronomical Society 202(3):615-627, 1983. doi.org/10.1093/mnras/202.3.615), which explicitly relies on D-max of the pair.

To ascertain the ability of the provided method to enable single molecule sequencing, a training set was created by conjugating specific amino acids to the DNA using the Edman chemistry. The modified DNA thus produced was sequenced using the R10 flow cells. The raw signal (squiggles) obtained from those experiments were processed using in-house built python scripts and open source libraries. Specifically, time-series feature extraction and supervised classification (Multilayer Percepteron; MLP) were performed on the training dataset. The table below shows the confusion matrix resulting from the analysis, showing 84% average accuracy in amino acid identification in a 2-way classification experiment between Glycine and Arginine.

Predicted

| True | Gly | Arg |
|---|---|---|
| Gly | 83 | 17 |
| Arg | 15 | 85 |

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (XXXIV) Exemplary Embodiments

1. A hybrid polymer including at least one repeat unit having the structure of any one of Formulae (1)-(X):

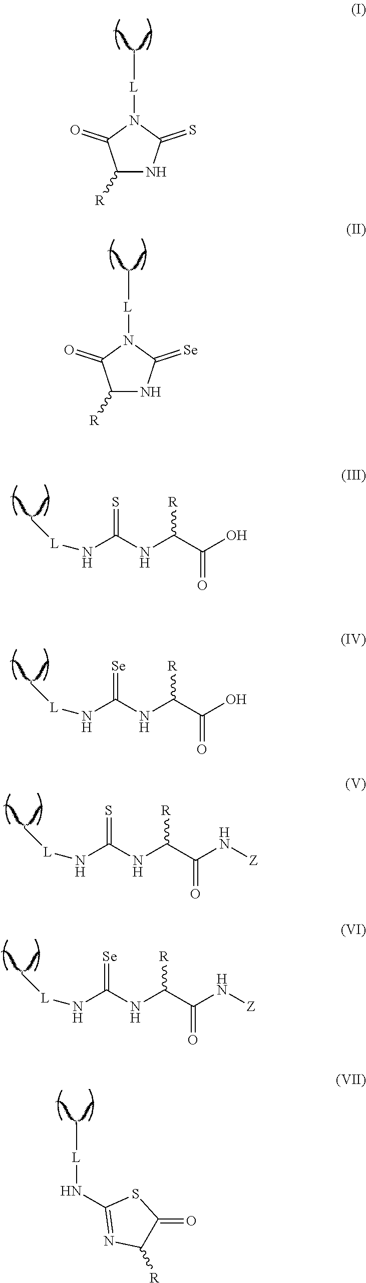

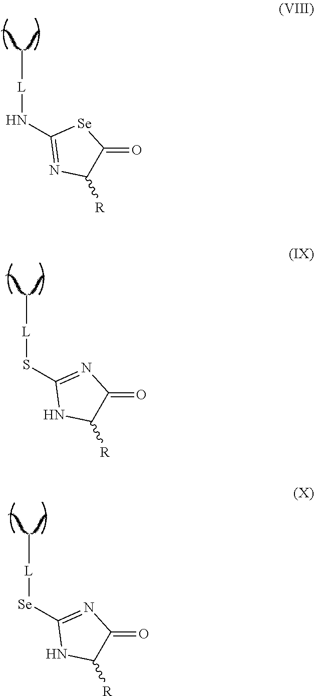

wherein the symbol “ [symbol] ” represents a single-stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein; [symbol] indicates that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid.
2. The hybrid polymer of embodiment 1, wherein the protection or the modification of the side chain is to minimize or prevent unwanted side reactions or to render the side chain more detectable than the unprotected or unmodified side chain.
3. The hybrid polymer of embodiment 1 or 2, wherein the natural or synthetic biopolymer includes repeat units of nucleic acids and/or amino acids.
4. The hybrid polymer of embodiment 3, wherein the natural biopolymer includes a polynucleotide.
5. The hybrid polymer of embodiment 4, wherein the polynucleotide comprises a natural or synthetic double-stranded DNA, a natural or synthetic double-stranded RNA, or a natural or synthetic polynucleotide comprising both DNA and RNA.
6. The hybrid polymer of any of embodiments 1-5, wherein the repeat units include at least two selected from the group of Formula (1), Formula (II), Formula (III), Formula (IV), Formula (IX), and Formula (X).
7. The hybrid polymer of embodiment 6, wherein the repeat units include at least two selected from the group of Formula (1), Formula (III), and Formula (IX).
8. A polymer including at least one repeat unit having the structure of any one of Formulae (XI)-(XX):

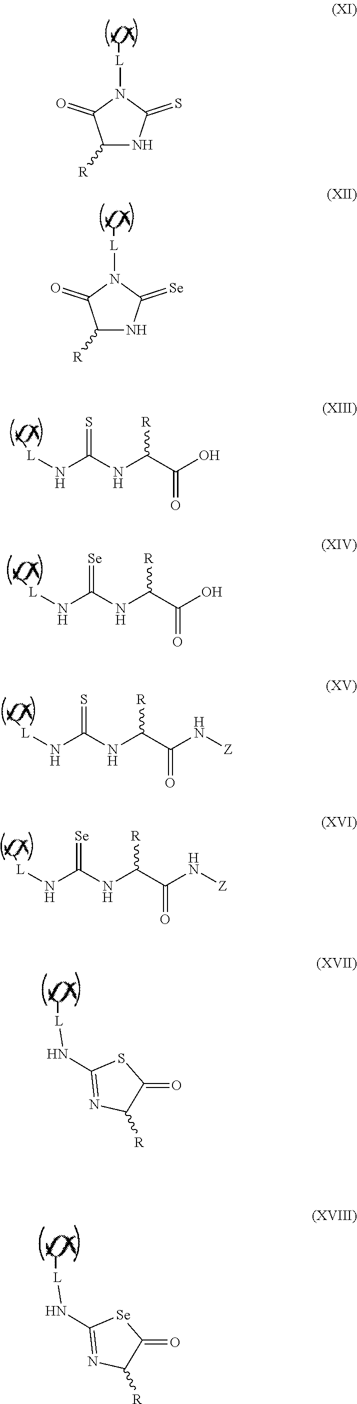

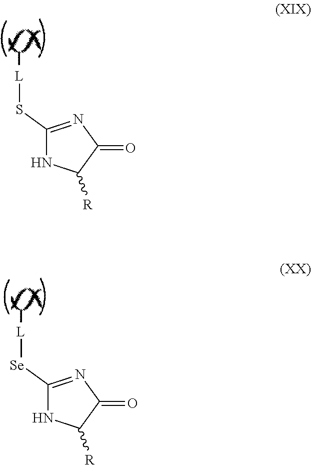

wherein the symbol “ [symbol] ” represents a double stranded natural or synthetic biopolymer; each R independently is the side chain, protected side chain or modified side chain of a (D)- or (L)-amino acid; L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein. L is the structure resulting from a reactive functionality on the AB or hybrid polymer and a reactive functionality on the NTAA of the peptide or resultant SCD. The structure includes any structures or spacers included in the creation of the reactive functionalities; [symbol] that the amino acid could be a (D)- or an (L)-amino acid; and each “Z” independently is hydrogen, a single amino acid, a peptide of 2 to 200 amino acids, or a protein of up to 2000 amino acids, wherein the amino acid is a natural, unnatural, or synthetic amino acid.
9. The hybrid polymer of embodiment 8, wherein the protection or the modification of the side chain is to minimize or prevent unwanted side reactions or to render the side chain more detectable than the unprotected or unmodified side chain.
10. The hybrid polymer of embodiment 8 or 9, wherein the natural of synthetic biopolymer includes repeat units of nucleic acids and/or amino acids.
11. The hybrid polymer of embodiment 10, wherein the natural biopolymer includes a polynucleotide.
12. The hybrid polymer of embodiment 11, wherein the polynucleotide comprises a natural or synthetic double-stranded DNA, a natural or synthetic double-stranded RNA, or a natural or synthetic polynucleotide comprising both DNA and RNA.
13. The hybrid polymer of any of embodiments 8-12, wherein the repeat units include at least two selected from the group of Formula (XI), Formula (XII), Formula (XIII), Formula (XVI), Formula (XIX), and Formula (XX).
14. The hybrid polymer of embodiment 13, wherein the repeat units include at least two selected from the group of Formula (XI), Formula (XII) and Formula (XIX).
15. A method of making a hybrid polymer, including: modifying a donor polypeptide having a C-terminal amino acid (CTAA) and an N-terminal amino acid (NTAA), such that in the modification, the adjacent amino acids of the donor peptide are separated by segments of a polymeric seed strand (PSS) having a distal end and a proximal end, and wherein in the hybrid polymer the identify and relative position of each amino acid of the donor polypeptide is retained.
16. The method of embodiment 15, wherein the modifying includes: (a) attaching the distal end of the PSS to a docking linker (DL) that connects the CTAA of the donor polypeptide and the PSS, and wherein the other end of the polymeric seed strand is the proximal end; (b) attaching an assembly block (AB) to the NTAA of the donor polypeptide by reaction between a reactive functionality in the NTAA and a reactive functionality in the AB; (c) ligating the AB to the proximal end of the polymeric seed strand; (d) cleaving the bond between the NTAA that is attached to the AB and the prior amino acid attached to the NTAA to expose a new NTAA; and optionally (e) repeating steps (b) through (d) at least once.
17. The method of embodiment 16, including repeating steps (b) through (d) until a plurality of amino acids in the donor peptide have been transferred to the hybrid polymer.
18. The method of any of embodiments 15-17, wherein the polymeric seed strand (PSS) includes a natural or synthetic biopolymer.
19. The method of embodiment 18, wherein the natural biopolymer include a polynucleotide.
20. The method of embodiment 19, wherein the polynucleotide includes a single stranded DNA (ssDNA) molecule.
21. The method of any of embodiments 16-20, wherein the docking linker (DL) includes a multifunctional DL, such as a bifunctional DL or a trifunctional DL.
22. The method of embodiment 21, wherein the bifunctional DL includes dibenzocyclooctyne-hexyl-N-succinimidyl (NHS) ester (DBCO-C6—NHS) having the structure:

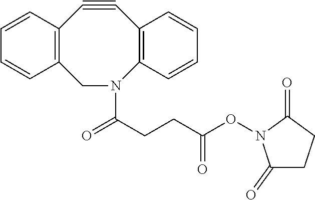 [SMILES: O=C(CCC(=O)N1Cc2ccccc2C#Cc2ccccc21)ON1C(=O)CCC1=O]

23. The method of embodiment 21, wherein the trifunctional DL includes amino, DBCO, and tetra C₁-C₄ alkoxysilane groups wherein the tetra C₁-C₄ alkoxysilane group is conjugated to a solid or semisolid support.
24. The method of embodiment 23, wherein the trifunctional DL has the structure:

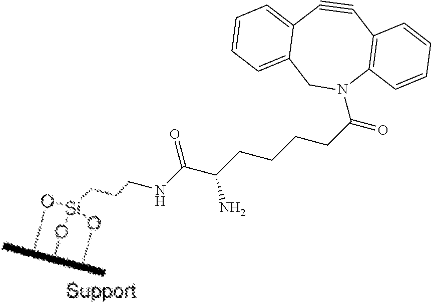 [SMILES: CNC(=O)[C@@H](N)CCCCC(=O)N1Cc2ccccc2C#Cc2ccccc21]

wherein the symbol “ [symbol] ” indicates a solid or semisolid support.
25. The method of any of embodiments 16-24, wherein the assembly block (AB) includes a polynucleotide having an isothiocyanate (—N═C═S) (ITC) or isoselenocyanate (—N—C═Se) (ISC) reactive functionality for reacting with the amino group of NTAA of the polypeptide.
26. The method of any of embodiments 16-25, wherein ligating the AB to the proximal end of the PSS includes an enzymatic or a non-enzymatic chemical ligation.
27. The method of embodiment 26, wherein enzymatic ligation includes, stick end ligation, blunt end ligation, splint ligation, or single-strand ligation.
28. The method of embodiment 26, wherein the non-enzymatic chemical ligation includes click chemistry.
29. The method of any of embodiments 16-28, wherein cleaving the bond between the NTAA that is attached to the AB and the prior amino acid attached to the NTAA to expose a new NTAA takes includes Edman degradation, Edman degradation enzyme reaction, or a similar process.
30. A hybrid polymer made by the method of any of embodiments 15-29.
31. A method of sequencing a donor polypeptide, including: preparing a hybrid polymer according to the method of any of embodiments 15-29; and analyzing the hybrid polymer, for instance wherein the analyzing includes identifying two or more amino acids of the donor polypeptide in order along the hybrid polymer.
32. The method of embodiment 31, wherein analyzing the hybrid polymer includes passing the hybrid polymer through a nanopore to sequentially identify single amino acids of the donor polypeptide, thereby sequencing the polypeptide.
33. The method of embodiment 32, wherein spacing of the amino acids along the hybrid polymer is sufficient to reduce, enhance, or otherwise alter (more generally, modify) electrical signal contribution of adjacent amino acids, thereby allowing nanopore-based peptide sequencing at single-amino acid resolution.
34. The method of embodiment 32 or embodiment 33, wherein the hybrid polymer is a polynucleotide, and wherein spacing of the amino acids along the hybrid polymer is sufficient to allow a helicase enzyme to slow down the rate and movement step size of the hybrid polymer through the nanopore.
35. The method of embodiment 31, wherein analyzing the hybrid polymer includes SMRT sequencing.
36. A kit for performing the method of any of embodiments 15-29 or 31-36, including: one or more polymeric seed strand (PSS), each having a distal end and a proximal end; and one or more assembly blocks, conjugation reagents, ligation reagents, Edman cleavage reagents, wash buffers, solid supports, listings of barcodes, and/or analysis software.
37. A method of sequencing a polypeptide, including: expanding distance between each amino acid of the polypeptide by attaching each amino acid in order to a (non-protein) polymer molecule to produce a hybrid polymer; and analyzing the hybrid polymer, for instance by passing the hybrid polymer through a nanopore to read a single amino acid at a time or by SMRT sequencing.
38. The method of embodiment 37, wherein the polymer molecule includes a nucleic acid backbone.
39. The method of embodiment 38, wherein the nucleic acid backbone is single stranded or double stranded.
40. The method of embodiment 39, wherein the amino acids of the polypeptide are attached to the polymer molecule: from the N-terminal end to the C-terminal end of the polypeptide; or from the C-terminal end to the N-terminal end of the polypeptide.
41. A method of sequencing a polypeptide, wherein the polypeptide has a C-terminal amino acid (CTAA) and an N-terminal amino acid (NTAA), the method including: producing a hybrid polymer by: (a) attaching a flexible linker to the CTAA of the polypeptide; (b) attaching an (isothiocyanate (ITC) or an analogue thereof)-DNA conjugate to the NTAA of the polypeptide; (c) aligning the end of the linker proximal to the NTAA and proximal to the end of the (ITC or an analogue thereof)-DNA conjugate though a bridge oligomer; (d) ligating the linker proximal to the NTAA and the end of the (ITC or an analogue thereof)-DNA; (e) cleaving the NTAA; (f) attaching an (ITC or an analogue thereof)-DNA conjugate to the new NTAA of the polypeptide; and (g) repeating steps (c) through (e) until the end of polypeptide is reached and there is no new NTAA left from the original polypeptide for attaching to an (ITC or an analogue thereof)-DNA conjugate; to generate a linearized and expanded polypeptide chain ready for sequencing; and analyzing the hybrid polymer to identify each amino acid of the donor peptide, thereby sequencing the polypeptide.

### (XXXV) EXPERIMENTAL EXAMPLES

### Example 1: Stability of DNA Under Acidic Conditions with Heating

The stability was tested of three oligonucleotide 96-mers differing by purine content, low purine (LP; SEQ ID NO: 1), high purine (HP; SEQ ID NO: 2), and no purine (NP; SEQ ID NO: 3), incubated in 1% v/v aqueous trifluoroacetic acid (TFA) for 24 h at 50° C. or 95° C. on a ProFlex PCR machine with heated lid. The controls were incubated at room temperature (21° C.) for 24 h without TFA also on the PCR machine with heated lid. Triethylamine was added to adjust each sample to a pH of approximately 8 to 8.5 and the samples were loaded onto a hand-cast, 24 cm long, 1 mm thick, 15% polyacrylamide tris-borate-ethylenediaminetetraacetic acid (EDTA)-urea denaturing sequencing gel. The gel (15% of 19:1 acrylamide:bis-acrylamide with APS and TEMED catalysis, 1× tris-borate EDTA, and 8 M urea) was run on a for a Hoefer SE660 apparatus at a constant 300 V for 12.5 h. The oligonucleotides were post-stained with 3× SYBR Gold and visualized on an Invitrogen iBright gel documentation system. Complete strand scission of LP and HP was observed at both 50° C. and 95° C. (no observed oligonucleotide or oligonucleotide fragments of approximately 5 or more bases). Complete strand scission of NP was observed at 95° C. (no observed oligonucleotide or oligonucleotide fragments of approximately 5 or more bases), but little to no strand scission was observed for NP in 1% TFA at 50° C. Results are shown in FIG. 10. These results indicate that DNA designs for the PSS and AB should limit the use of canonical purine bases (A, G) which are more likely to undergo depurination and strand scission compared to pyrimidine bases (C, T).

### Example 2: Making and Sequencing Hybrid Polymer/PROSE Constructs

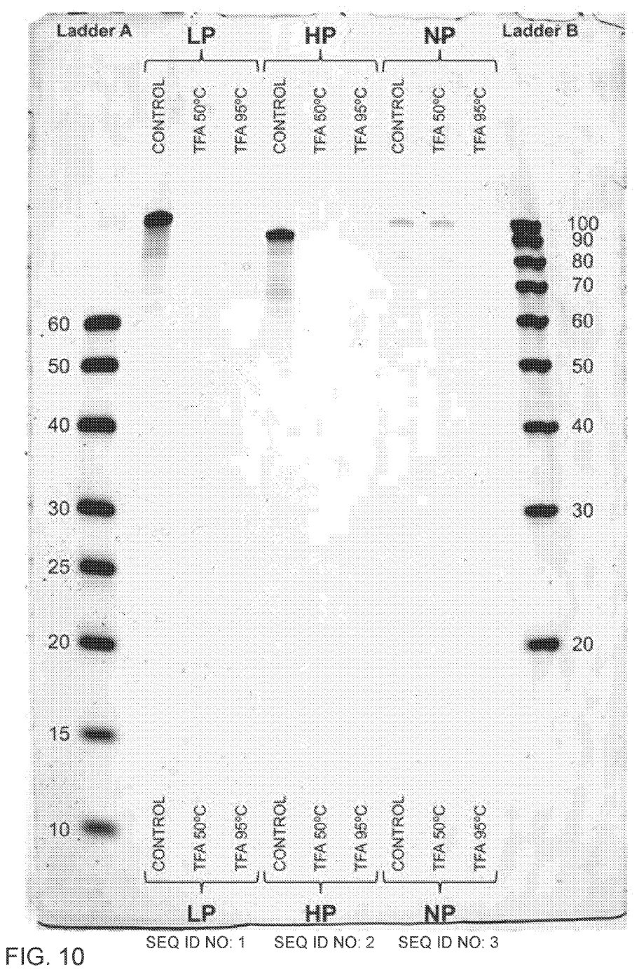

An example of the PROSE workflow was performed with samples of partially-assembled PROSE constructs taken at four different intervals (FIG. 11, left gel panel): (AB₁, SEQ ID NO: 5) supernatant of the Edman degradation reaction (E.D. Sup.) after ligation of the first assembly block (AB) in the workflow; (AB₂, SEQ ID NO: 6) supernatant of the Edman degradation reaction after ligation of the second conjugated oligo, AB₂, in the workflow; and (PSS-AB₁, SEQ ID NOs: 4-5) substrate cleavage after the first assembly cycle to form PSS-AB₁, (PSS-AB₁-AB₂, SEQ ID NOs: 4-5-6) substrate cleavage after the second assembly cycle to form PSS-AB₁-AB₂. These fractions were run on a 15% tris-borate-urea gel to validate proximity ligation of the AB₁-peptide (AB₁-TU-DP) conjugates. Both ligated products (extensions) and impurities were retained in the PSS-AB₁ and PSS-AB₁-AB₂ samples, and the final construct yielded sequencing results, as depicted in FIG. 18 and FIG. 19.

To confirm peptide conjugation occurred in the absence of AB-AB dimers, a mock reaction was performed in suspension. The mock substrate-bound seed strand depicts a different length profile than AB₁-ITC. When AB₁-ITC is exposed to peptides in solution at pH 8.5 (0.1% v/v triethylamine in water), an oligo-peptide conjugate band is present above the 40 base ladder mark (AB₁-Peptide). To control for potential dimer formation between ABs, the same reaction was performed without peptides. Some dimers may have formed but are obfuscated by impurities in the oligonucleotide solution (AB₁-dimer).

### Example 3: Cleavage of NTAA from AB-Peptide Conjugates

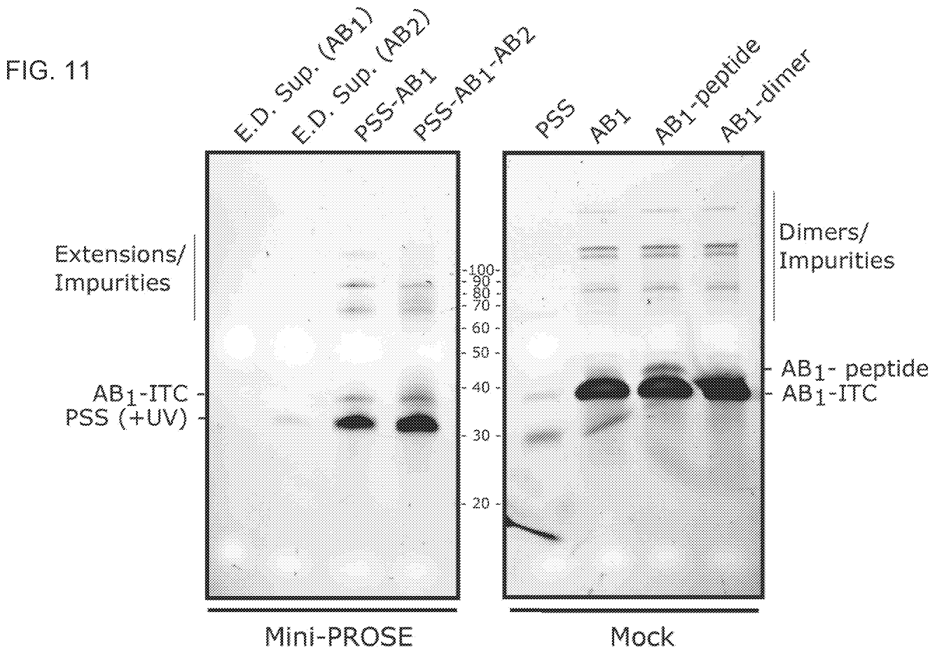

AB-peptide conjugates (Mock) were created as described herein. Briefly, 1 nmol of AB-ITC (SEQ ID NO: 5 or SEQ ID NO: 6) was mixed with an equivalent amount of peptide in the presence of 0.1%% v/v triethylamine in pure molecular grade water. Reactions were run for 1 hour at 50 degrees Celsius. Samples were buffer exchanged into pure water and 0.5, 1 and 2% v/v trifluoroacetic acid (TFA) or trichloroacetic acid (TCA) and run for 30 minutes or overnight. The final products were run on a 15% tris-borate urea denaturing polyacrylamide gel and stained with SYBR Gold for visualization to determine the conjugation yield and subsequent Edman degradation efficiency. FIG. 12 illustrates the gel electrophoresis analysis of the assembly block-thiourea-donor peptide (AB-TU-DP) conjugates under various Edman degradation conditions. Control band (Cntl) portrays the AB-ITC without peptide present. Gel results indicate that AB-peptide conjugates can be cleaved to form AB-SCD with both TFA and TCA under the tested conditions.

### Example 4: Quantitation of Cleavage Efficiency

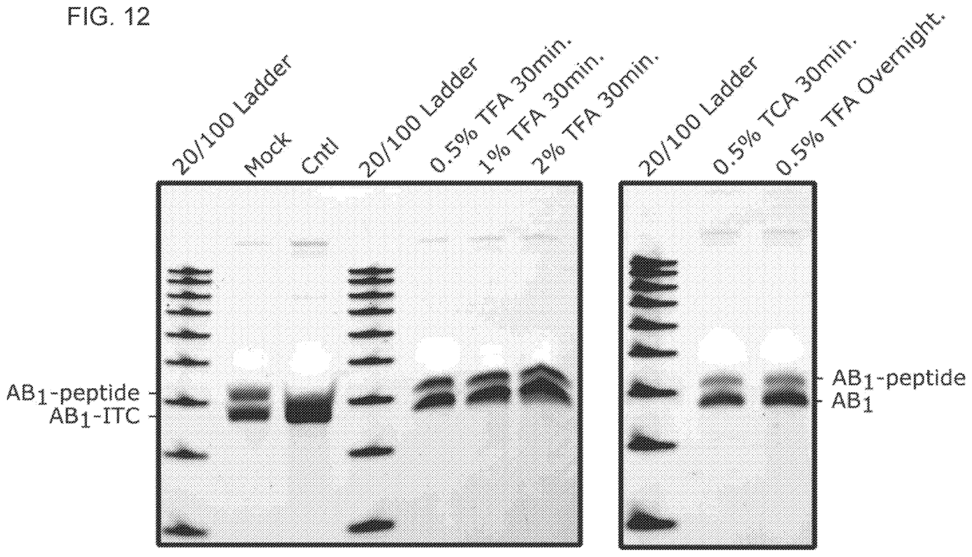

AB-peptide conjugates, cleavage reactions, and gel electrophoresis were performed as described in Example 3. herein. Densitometry of relative depletion of assembly block (AB)-TU-peptide conjugates after Edman degradation were calculated in FIJI, ImageJ to assess the fluorescence density of each of the bands. Relative depletion results are indicative of the successful cleavage of the N-terminal amino acid (NTAA), although with low efficiency. Cleavage was further improved with higher percentages of TFA and longer reaction times. The results are illustrated in FIG. 13.

### Example 5: Exemplary Methods to Conjugate AB-ITC to a Peptide

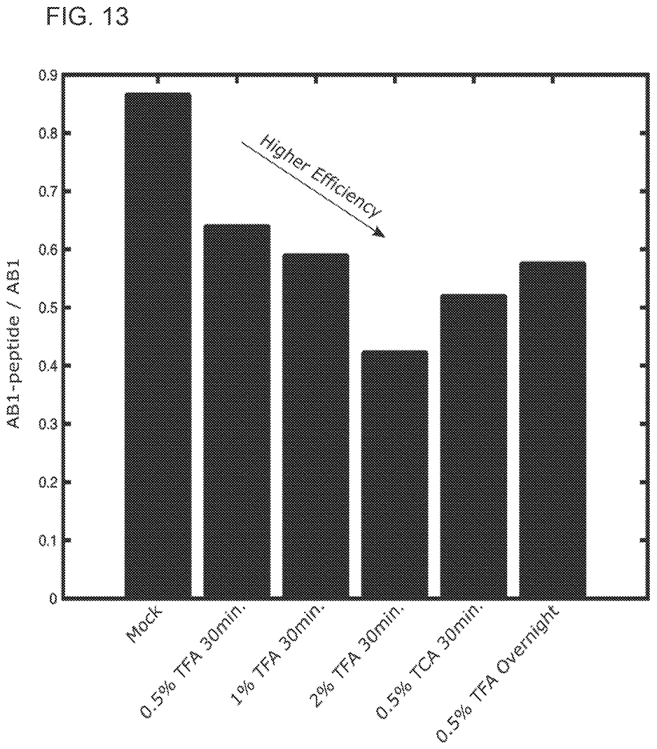

Various examples of conjugation of an assembly block-isothiocyanate (AB-ITC) conjugation to a peptide are illustrated in FIG. 14. In the left panel, azide-PEG3-isothiocyanato was conjugated to 1 nmol of peptides in 0.1% v/v triethylamine in pure molecular grade water. 1 nmol AB (SEQ ID NO: 5 or SEQ ID NO: 6) was conjugated to NHS-DBCO in pure water. The resulting molecules were mixed for Azide-DBCO click chemistry as an alternative to the AB-ITC conjugation strategy. The resulting conjugates were at similar yield to other embodiments. To further validate conjugation of AB-ITC to peptides, a DBCO-AF488 dye was conjugated to a peptide containing an azido-lysine at the C-terminus. This peptide was then conjugated to the AB-ITC strand as stated previously in 0.1% triethylamine. The fluorescence of AF488 was observed in bands with the same molecular weight as those observed by SYBR Gold staining. Furthermore, Edman Degradation was carried out in 1% TFA and 100 mM Ag Triflate Lewis Acid to assess differences between the two forms of acid. Both TFA and Ag Triflate had marginal efficiency. Degradation results indicate that a Lewis acid can be used for Edman degradation of the NTAA under optimal conditions.

### Example 6: Stability of DNA Under Acidic Conditions

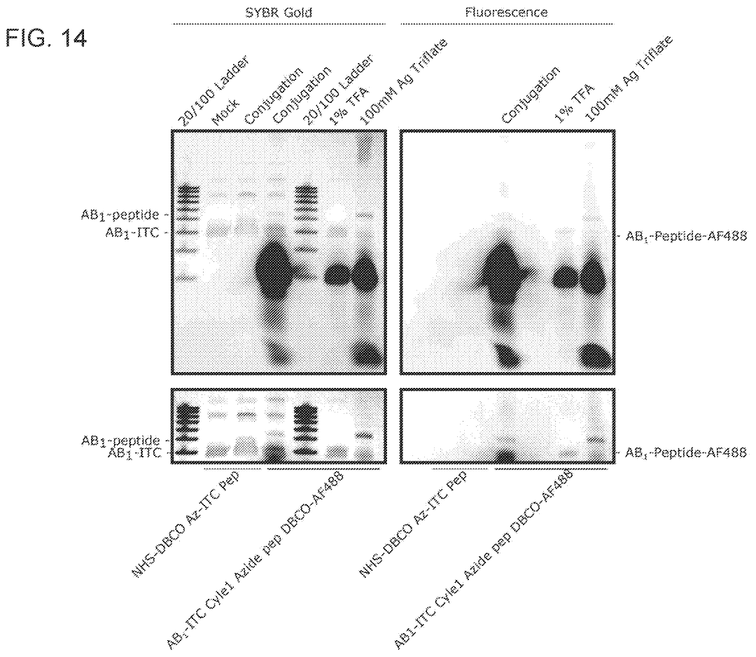

Edman degradation after AB-ITC (SEQ ID NO: 5 or SEQ ID NO: 6) conjugation was carried out with the following conditions for 30 minutes at 50° C.: 10% v/v 12N HCl in pure water, 0.5% v/v Trichloroacetic acid (TCA) in pure water, 25% v/v Trichloroacetic acid in pure water. Reactions were run on a 15% tris-borate urea polyacrylamide gel and stained with SYBR Gold to visualize bands. The resulting bands (FIG. 15) indicated that HCl and TCA were both able to cleave AB-peptide through Edman Degradation. The higher percentage of TCA (25% v/v) completely degraded the AB in the reaction mixture.

In another example, varying acid conditions were used to generate a PROSE construct for sequencing. For Edman Degradation conditions 8%, 12% and 16% v/v trifluoroacetic acid in water, total mols of PROSE construct sequenced was measured to be 2.01 fmol, 2.58 fmol and 4.19 fmol, respectively. These showed a marked increase in product over the negative control, approximately 0.1 fmol. The PROSE construct utilizing 8% v/v aqueous trifluoroacetic acid for Edman Degradation was also analyzed by capillary electrophoresis, indicating a 20-fold increase over the negative control.

### Example 7: Quantification of Cleavage Efficiency

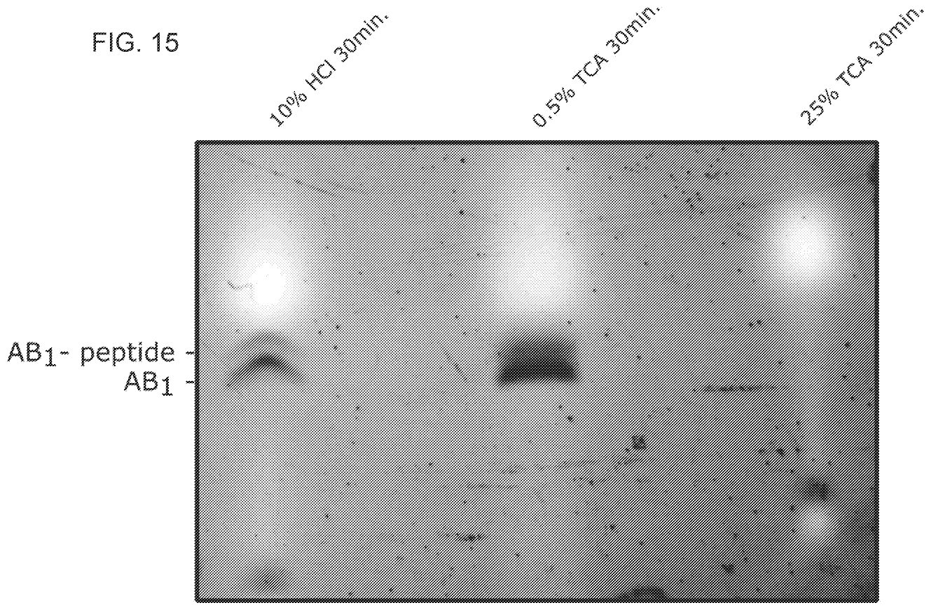

Densitometry of peptide oligo-conjugate depletion after alternative Edman degradation strategies was calculated by taking the integrated intensity of the stained fluorescent regions in FIJI, ImageJ and dividing the AB-peptide density over the AB-ITC density. Both 0.5% TCA and 10% v/v HCl had similar cleavage efficiencies, as illustrated in FIG. 16. Results indicate that Edman degradation of the NTAA can occur in various acid conditions.

### Example 8: Building PROSE

This example illustrates an embodiment of PROSE.

In this example, in step (a) the distal end of the PSS was attached to a docking linker (DL) that connects the CTAA of the donor polypeptide (SEQ ID NOs: 26-30) and the PSS, and wherein the other end of the polymeric seed strand is the proximal end. The docking linker was streptavidin attached to nanoparticles or was chemical linker(s) attached to oligonucleotide(s) (e.g., PSS in Table 1). About 50-500 pmole of polypeptide and oligonucleotides were used.

In step (b), the first assembly block (AB1) was attached to the NTAA of the donor polypeptide by reaction between the amino group of the NTAA and a reactive functionality in the AB1; the amino group of the AB1 (SEQ ID NO: 5, P003 in Table 1) was converted to isothiocyanate (ITC) as described in FIG. 23. 1-5 nmole of AB1 was mixed with polypeptide and oligonucleotides from the previous step in a PBS buffer which pH had been adjusted to 8.5 by triethylamine (Et₃N). The reaction took 1 hour, and the mix was washed thrice with PBS before the next step.

In step (c), 1 nmole of Splint (SEQ ID NO: 14, P030 in Table 1) was mixed with 50 μL of 1× NEB 2 buffer and used to resuspend the sample. After 3 mins, the sample was resuspended in a 50 μL ligation reaction buffer prepared by using Quick Ligation Kit (M2200S, NEB), and the P001 and AB1 was ligated after 10-15 mins in room temperature. The sample was washed several times with PBS with 0.1% Tween 20.

In step (d), the sample was washed with ultrapure water 3 times then suspended in 0.5%-1% TFA in water which cleaved the bond between the NTAA that is attached to the AB1 and the prior amino acid attached to the NTAA to expose a new NTAA.

Steps (b) through (d) were repeated by alternating the combination of assembly block (SEQ ID NO: 6, AB2)/Splint(SEQ ID NO: 15, P031) and assembly block (SEQ ID NO: 5, AB1)/Splint(SEQ ID NO: 14, P030).

The sample was then ligated to a primer (SEQ ID NO: 7, P007 or other more specific primer SEQ ID NO: 16-25, P020-P029), followed by second strand synthesis by Klenow Fragment (3′--*5′ exo-) (M0212S, NEB)) for 30 mins under 37° C.

The sample was washed 3 times with ultrapure water and was exposed with 6W 365 nm UV lamp for 30 mins to cleave the e linker on PSS and release the double stranded oligonucleotides.

Finally, 30-100 fmole of the photocleaved sample was end-repaired and ligated to nanopore sequencing adapter by following library preparation protocol either Amplicon-LSK110 or Amplicon LSK-114 from Oxford Nanopore Technology (ONT) for nanopore sequencing flow cell R9.4.1 or R.10 respectively. The samples were sequenced on a GridlON, and the sequencing data of fastq and fast5 files were analyzed by GridlON built-in basecaller and customized algorithm.

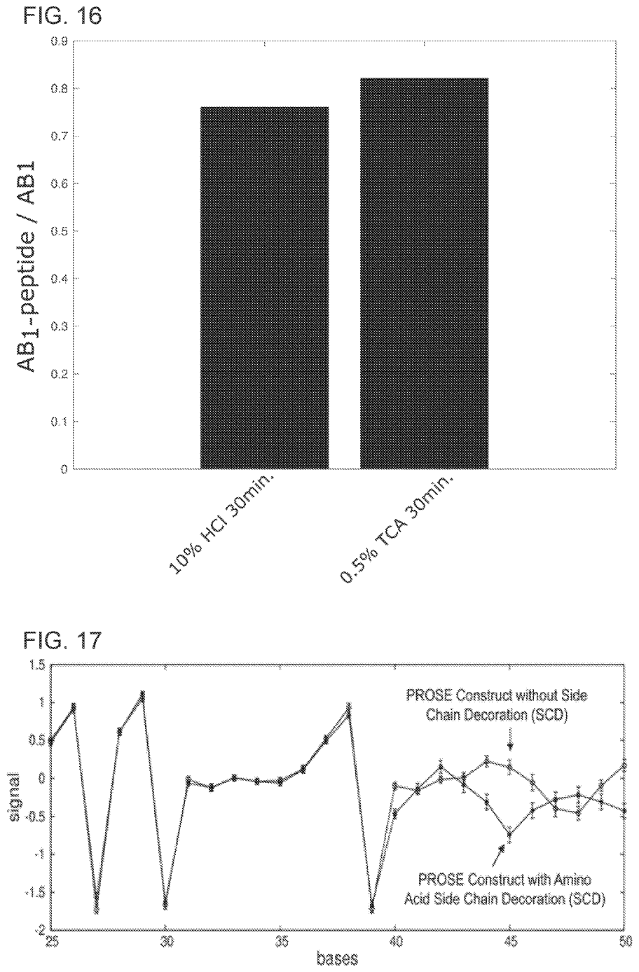

FIG. 17 illustrates the averaged raw nanopore signal showing differential signal response according to the presence or absence of a SCD on the PROSE construct obtained using this sample preparation on SEQ ID NO: 1.

FIG. 18 illustrates the number of nanopore reads of PROSE constructs assembled with (solid bars) or without (hollow bars) donor peptide, from the sample preparation described in this Example on Construct with SEQ ID NOs: 4, 5 & 6.

FIG. 19 Sequence alignment of reads of the PROSE construct produced in this example containing SEQ ID NOs: 4, 5 & 6. Gaps in the alignment correspond to the presence of an amino acid side chain decoration (SCD) borne by the PROSE construct.

### Example 9: Building PROSE Single Amino Acid Data without Edman Degradation

This example illustrates another embodiment of PROSE, which does not rely on Edman degradation.

In this example, in step (a) the distal end of the PSS was attached to a docking linker (DL).

The docking linker was streptavidin attached to nanoparticles or was a chemical linker attached to an oligonucleotide (e.g. PSS in Table 1). About 50 nmole of oligonucleotides were used.

In step (b), the first assembly block (AB1) was conjugated to single amino acid (SEQ ID 31-34) by reaction between the amino group of the single amino acid and a reactive functionality in the AB1; the amino group of the AB1 (SEQ ID NO: 5, P003 in Table 1) was converted to isothiocyanate (ITC) as described in FIG. 23. 1-5 nmole of AB1 was mixed with 50-1000 nmole of single amino acid (e.g., Histidine, Arginine, Tryptophan, Glycine) in a PBS buffer which pH had been adjusted to 8.5 by triethylamine (Et₃N). The reaction was run for 12 hours, and the mix was washed thrice with PBS before the next step.

In step (c), 1 nmole of Splint (P030 in Table 1) was mixed with 50 μL of 1× NEB 2 buffer and used to resuspend the sample. After 3 mins, the sample was resuspended in a 50 μL ligation reaction buffer prepared by using Quick Ligation Kit (M2200S, NEB), and the P001 (SEQ ID NOs: 4 and 62; in two parts, before and after the linker) and AB1 was ligated after 10-15 mins at room temperature. The sample was washed few several times with PBS with 0.1% Tween 20.

Steps (b) through (d) were repeated by alternating the combination of assembly block (SEQ ID NO: 6, AB2)/Splint (SEQ ID NO: 15, P031) and assembly block (SEQ ID NO: 5, AB1)/Splint (SEQ ID NO: 14, P030). AB2 can be an oligonucleotide with only canonical group.

The sample was ligated to a primer (P007 or other more specific primer P020-P029 SEQ ID NO: 7, P007 or other more specific primer SEQ ID NOs: 16-25, P020-P029), followed by second strand synthesis by Klenow Fragment (3′--*5′ exo-) (M0212S, NE) for 30 mins under 37° C.

The sample was washed 3 times with ultrapure water and was exposed with 6W 365 nm UV lamp for 30 mins to cleave the photocleavable linker on PSS and release the double stranded oligonucleotides.

Finally, 30-100 fmole of the photocleaved sample was End-repaired and ligated to nanopore sequencing adapter by following library preparation protocol either Amplicon-LSK110 or Amplicon LSK-114 from Oxford Nanopore Technology (ONT) for nanopore sequencing flow cell R9.4.1 or R.10 respectively. The samples were sequenced on a GridlON, and the sequencing data of FASTQ and FAST5 files were analyzed by GridlON built-in basecaller and customized algorithm.

### Example 10: Direct Production of Multifunctional Linkers from a Solid or Semi-Solid Support

Multifunctional docking linkers (DLs) can be directly synthesized onto solid or semi-solid supports using standard methods of solid phase peptide synthesis (SPPS) or solid phase oligonucleotide synthesis (SPOS). These methods can be used with amino acids, peptide nucleic acids, nucleic acids, spacers, and terminal modifications of natural, unnatural, and/or synthetic origin. Solid or semi-solid supports can include surfaces; particles, such as nanometer-, micrometer-, or millimeter-sized particles; and other shapes and forms of matter. These solid or semi-solid supports can be composed of materials including polystyrene, metal, iron oxide, tentagel, glass, ceramics, hydrogel, polymer, and/or plastic and can also have porosity varying from non-porous to porous.

Monomer, spacer, or terminator used in SPPS or SPOS can include reactive functional groups such as amines, hydrazine, azides, N-hydroxysuccinimide (NHS), dibenzocyclooctyne (DBCO), alkynes, tetrazine, maleimide, aldehyde, acrylate, TCO, thiol, and alkenes. Photo-reactive crosslinkers, such as photo-reactive azides (phenyl azide, ortho-hydroxyphenyl azide, meta-hydroxyphenyl azide, tetrafluorophenyl azide, ortho-nitrophenyl azide, meta-nitrophenyl azide, and azido-methylcoumarin), diazirine, and psoralen. Photocleavable monomers or spacers, such as those including nitrophenyl groups, can also be included to facilitate the controlled cleavage of the PROSE construct and/or other polymers or assemblies from the support. Reactivity of the various reactive groups can be directionally controlled via standard blocking/protecting group strategies used in SPPS and SPOS and by taking advantage of high yield reactions, including click chemistry.

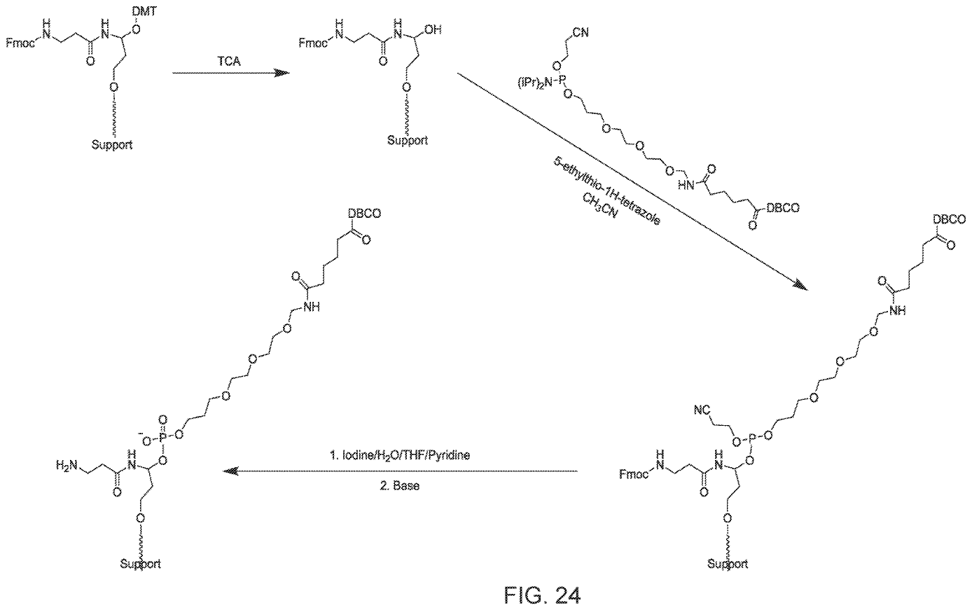

One example of a multifunctional linker produced via SPOS is shown in FIG. 24.

### Example 11—Single Molecule Real Time Sequencing of PROSE Constructs

An alternative platform that is compatible for sequencing PROSE constructs/hybrid polymers as described herein is offered by Pac Bio's SMRT instruments, Sequel 1/II and Revio.

The technology detects polymerase incorporation of the cognate base. The kinetic characteristics, such as the time duration between two successive base incorporations, is altered by the presence of a modified base in the DNA template. This is observable as an increased space between fluorescence pulses, which is called the interpulse duration (IPD). The SCD on the hybrid polymer will provide a signature unique to the identity of the amino acid, thus enabling sequencing of PROSE constructs using SMRT instruments.

By way of example, PROSE constructs (hybrid polymers) are made according to Example 8 up to photocleavage of the PSS, or Example 9 up to end repair. Pac Bio library prep kits such as SMRTBELL library prep for hifi sequencing (PN:102-182-700) are then used (per package insert) to create a circular consensus sequencing (CCS) template. That template will contain unmodified AB blocks to serve as an internal control, AB with linker, and several AB-SCD. Sequencing by SMRT will demonstrate that SCDs do affect polymerase kinetics as measured by IPD and also that the SCDs cause mis-incorporation errors that will aid in the identification of each amino acid.

SMRT sequencing expands the utility of PROSE and may be useful for larger PTMS that may be too large for some nanopores. Initial results may be optimized through choice of linkers (length, type, etc.), sequence context around SCD, and protecting or blocking groups on the SCD.

### Example 12—PROSE Based Long or Linked Reads

As in DNA sequencing, long read methods are necessary for discovery or scaffolding of short reads. In proteomics, a similar need exists. An efficient PROSE chemistry will be able to be able to create a construct or hybrid polymer with 100 or more SCDs. However, as growing PSS becomes larger, the proximity effect for ligation will likely suffer due to degrees of freedom and/or steric issues. It will be advantageous to remove the PROSE strand and continue the PROSE chemistry until the donor protein is converted to a hybrid polymer. Transferring a UMI to multiple PROSE strands from the same protein is contemplated in this circumstance. Synthetic long reads use multiple PSS strands that are encoded so that the order of PROSE sequences could be digitally reconstructed.

PROSE cycle will be performed as described in Examples 8 & 9. The first PSS will have additional binding sequences for subsequent PSS strands. At the stopping point for hybrid polymer-1, a hairpin or double-stranded AB containing a UMI is conjugated to the NTAA. Then the dsAB will be ligated to the PSS but unlike the previous cycles, the AB strand that will be ligated does not contain the SCD. The SCD will be on the reverse complement strand. The hybrid polymer-1 will be photocleaved as described herein. This construct will contain the original PSS barcode and a terminating UMI.

A second PSS (PSS-2) will be attached to a region on the first PSS (that remains after photocleavage) and will be covalently linked for instance through T dimers or 3-Cyanovinylcarbazole photocrosslinker. PSS-2 will be identifiable as the second PSS by sequencing. PROSE will restart by ligation of the UMI reverse complement to PSS-2. This example will demonstrate two cycles of PROSE and that the PROSE strands is identifiable using this UMI strategy.

Linked reads will follow similar processes, but there will be gaps in the sequence because the protein will be shortened by chemical or enzymatic methods. Overlapping sequence alignments will be able to reconstruct the continuous sequence.

### Example 13—Training Data for Single Amino Acid Identification

Single cycle PROSE used to develop datasets to train machine learning models.

Exemplary Peptides (SEQ ID NOs: 58, 60, 65, 66, 68) are covalently linked to magnetic beads (Dynabeads™ M-270 Amine, Thermo Fisher Scientific) via azide-DBCO linkage. Assembly block (AB) (SEQ ID NOs: 12 and 63; before and after the linker) was attached to the NTAA of the donor polypeptide by reaction between the amino group of the NTAA and a reactive group on AB; the amino group of the AB (SEQ ID NOs: 12 and 63, P040) was previously converted to isothiocyanate (ITC) to form AB-ITC. 0.1-5 nmole of AB-ITC was mixed with the magnetic beads from the previous step in PBS buffer adjusted to pH 8.5-9 by triethylamine (Et3N). The reaction proceeded for 6-15 hours, and the mix was washed thrice with PBS before the next step. 900 pmole of Splint (SEQ ID NO: 68, P115) was mixed with 150 μL of 1× NEB 2 buffer and used to resuspend the sample. After 3 mins, the buffer was aspirated and the sample was resuspended in a 100 μL ligation reaction buffer prepared by using Quick Ligation Kit (M2200S, NEB) with added P080 (SEQ ID NOs: 69 and 70). AB-ITC was ligated to P080 for 15-20 mins at room temperature. The sample was washed thrice with PBS supplemented with 0.1% Tween 20 and subsequently Edman degraded as described to cleave the oligonucleotide-conjugated NTAA. The magnetic beads were transferred to a post Edman condition washing buffer composed of 150 μL of PBS supplemented with 0.1% Tween 20. A 5 μL volume of streptavidin beads (Dynabeads™ MyOne™ Streptavidin C1) was added to the post-Edman degradation solution to capture cleaved products carrying biotin on the P080 oligonucleotide 5′ termini. The streptavidin beads were then transferred to the post Edman condition washing buffer for 30 mins to capture samples released from the beads during the post-Edman wash. The streptavidin beads carrying the P080-AB-ITC-NTAA construct were mixed with 900 pmole of Splint (SEQ ID NO: 15, P031 in Table 1) for annealing to the AB oligonucleotide, followed by ligation of oligonucleotides C1 to C12, which carry 12 distinct barcodes for multiplexing (SEQ ID NOs: 71 and 72, 71 and 73, 71 and 74, 71 and 75,71 and 76,71 and 77,71 and 78,71 and 79,71 and 80,71 and 81,71 and 82, and 71 and 83; pairs referring to sequences of two parts of each oligonucleotide, before the linker and after the linker respectively in each pair). The resulting construct was further ligated to Gblock 1 and 2 (SEQ ID NO: 85 and 86) via Splint P145 (SEQ ID NO: 84). The final product was resuspended in 15 μL of deionized water and cleaved from the beads by exposing the mixture to 365 nm UV for 30 mins. The cleaved samples were supplemented with 2 mM Mg²⁺ and annealed to 5-200 pmol of complementary strand of P080 (SEQ ID NO: 87), leaving a 3′ A-overhang for downstream T-A overhang ligations. The resulting construct can be processed by ONT Library Kit (LSK114) and sequenced. This process can be repeated on the same peptide beads to yield a 2^{nd} round of PROSE to assess multi-cycle efficiency.

In this example, constructs carrying NTAAs Arg, Leu, Trp, Gly can be sequenced to create datasets for machine learning models for Arg, Leu, Trp, and Gly.

In another example of using peptide SEQ ID: 56, R is the NTAA and G is the N-1 AA. Two rounds of single cycle PROSE were conducted and sequenced on the PromethlON nanopore sequencers. The results were analyzed using a machine learning algorithm to identify the total number of NTAA and N-1 AA from each round of experiments.

TABLE 2
Number of identified NTAA and N-1 AA from
multiple rounds of single cycle PROSE

| | | Number of sequenced PROSE |
|---|---|---|
| | | constructs with identified |
| | amino acid | amino acid |
| 1^{st} round of PROSE | NTAA/Arg | 13.3 femtomoles |
| 2^{nd} round of PROSE | N-1 AA/Gly | 0.53 femtomoles |

### (XXXVI) Closing Paragraphs.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically significant reduction in the ability to distinguish or identify amino acids using a PROSE protocol.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; +19% of the stated value; ±18% of the stated value; +17% of the stated value; +16% of the stated value; ±15% of the stated value; +14% of the stated value; ±13% of the stated value; +12% of the stated value; +11% of the stated value; +10% of the stated value; ±9% of the stated value; ±8% of the stated value; +7% of the stated value; ±6% of the stated value; ±5% of the stated value; +4% of the stated value; ±3% of the stated value; +2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A hybrid polymer comprising at least one repeat unit having the structure of any one of Formulae (1)-(X): 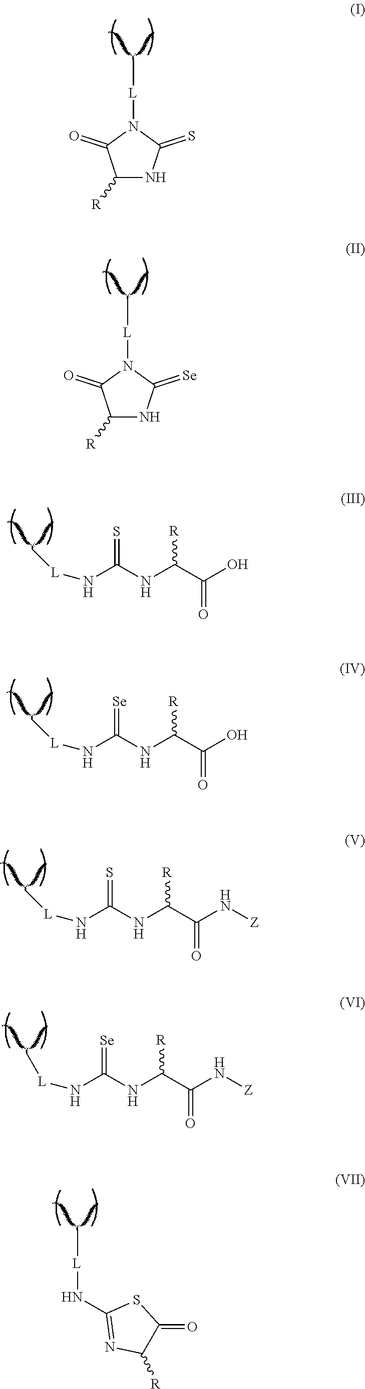 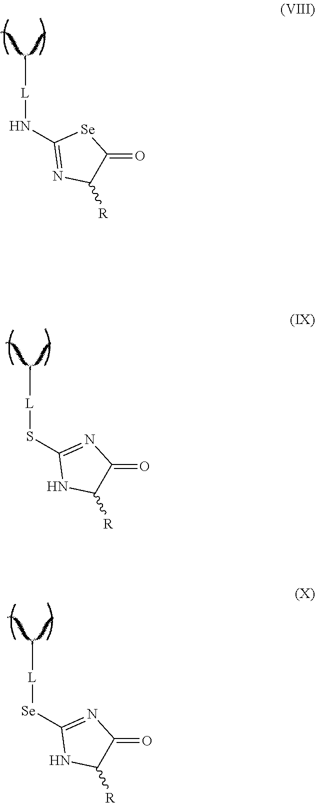
wherein the symbol “ [symbol] ” represents a single-stranded natural or synthetic biopolymer;
each R independently is the side chain, protected side chain or modified side chain of a (D)- or (L)-amino acid;
L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein; [symbol]
indicates that the amino acid could be a (D)- or an (L)-amino acid; and
each “Z” independently is hydrogen, a single amino acid, a peptide of 2 to 200 amino acids, or a protein of up to 2000 amino acids, wherein the amino acid is a natural, unnatural, or synthetic amino acid.

2. The hybrid polymer of claim 1, wherein the protection or the modification of the side chain is to minimize or prevent unwanted side reactions or to render the side chain more detectable than the unprotected or unmodified side chain.

3. The hybrid polymer of claim 1 or 2, wherein the natural or synthetic biopolymer comprises repeat units of nucleic acids and/or amino acids.

4. The hybrid polymer of claim 3, wherein the natural biopolymer comprises a polynucleotide.

5. The hybrid polymer of claim 4, wherein the polynucleotide comprises a natural or synthetic double-stranded DNA, a natural or synthetic double-stranded RNA, or a natural or synthetic polynucleotide comprising both DNA and RNA.

6. The hybrid polymer of any of claims 1-5, wherein the repeat units comprise at least two selected from the group of Formula (1), Formula (II), Formula (III), Formula (IV), Formula (IX), and Formula (X).

7. The hybrid polymer of claim 6, wherein the repeat units comprise at least two selected from the group of Formula (1), Formula (III), and Formula (IX).

8. A hybrid polymer comprising at least one repeat unit having the structure of any one of Formulae (XI)-(XX): 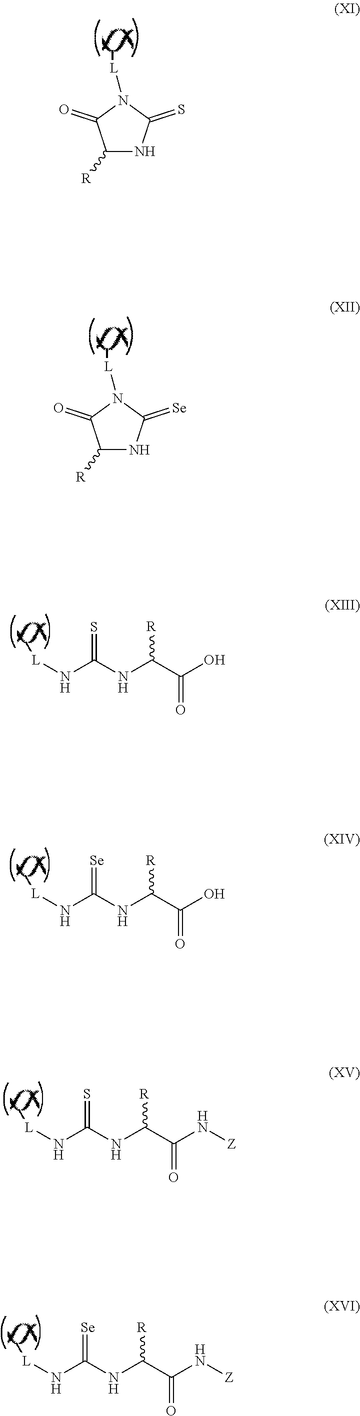 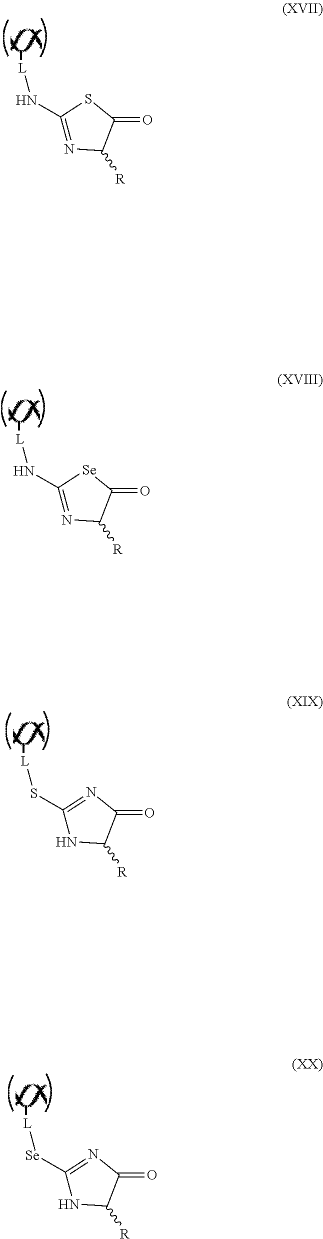
wherein the symbol “ [symbol] ” represents a double stranded natural or synthetic biopolymer;
each R independently is the side chain, protected side chain or modified side chain of a (D)- or (L)-amino acid;
L is a linker group linking the structure that follows it to adenine, thymine, guanine, cytosine, or uracil ring; or to the phosphate backbone —O—P(═O)—O—CH₂— that is between two sugar moieties in a nucleotide structure; or to a sugar moiety; or to a synthetic spacer; or to a non-natural or synthetic nucleobase or backbone structure; or to any structure contained within the definitions of “nucleic acid molecule” or “polynucleotide” herein, L is the structure resulting from a reactive functionality on the AB or hybrid polymer and a reactive functionality on the NTAA of the peptide or resultant SCD, The structure includes any structures or spacers included in the creation of the reactive functionalities; [symbol]
indicates that the amino acid could be a (D)- or an (L)-amino acid; and
each “Z” independently is hydrogen, a single amino acid, a peptide of 2 to 200 amino acids, or a protein of up to 2000 amino acids, wherein the amino acid is a natural, unnatural, or synthetic amino acid.

9. The hybrid polymer of claim 8, wherein the protection or the modification of the side chain is to minimize or prevent unwanted side reactions or to render the side chain more detectable than the unprotected or unmodified side chain.

10. The hybrid polymer of claim 8 or 9, wherein the natural of synthetic biopolymer comprises repeat units of nucleic acids and/or amino acids.

11. The hybrid polymer of claim 10, wherein the natural biopolymer comprises a polynucleotide.

12. The hybrid polymer of claim 11, wherein the polynucleotide comprises a natural or synthetic double-stranded DNA, a natural or synthetic double-stranded RNA, or a natural or synthetic polynucleotide comprising both DNA and RNA.

13. The hybrid polymer of any of claims 8-12, wherein the repeat units comprise at least two selected from the group of Formula (XI), Formula (XII), Formula (XIII), Formula (XVI), Formula (XIX), and Formula (XX).

14. The hybrid polymer of claim 13, wherein the repeat units comprise at least two selected from the group of Formula (XI), Formula (XII) and Formula (XIX).

15. A method of making a hybrid polymer, comprising:
modifying a donor polypeptide having a C-terminal amino acid (CTAA) and an N-terminal amino acid (NTAA), such that in the modification, the adjacent amino acids of the donor peptide are separated by segments of a polymeric seed strand (PSS) having a distal end and a proximal end, and wherein in the hybrid polymer the identify and relative position of each amino acid of the donor polypeptide is retained.

16. The method of claim 15, wherein the modifying comprises:
(a) attaching the distal end of the PSS to a docking linker (DL) that connects the CTAA of the donor polypeptide and the PSS, and wherein the other end of the polymeric seed strand is the proximal end;
(b) attaching an assembly block (AB) to the NTAA of the donor polypeptide by reaction between a reactive functionality in the NTAA and a reactive functionality in the AB;
(c) ligating the AB to the proximal end of the polymeric seed strand; and
(d) cleaving the bond between the NTAA that is attached to the AB and the prior amino acid attached to the NTAA to expose a new NTAA.

17. The method of claim 16, wherein the modifying further comprises:
(e) repeating steps (b) through (d) at least once.

18. The method of claim 16 or 17, comprising repeating steps (b) through (d) until a plurality of amino acids in the donor peptide have been transferred to the hybrid polymer.

19. The method of any of claims 15-18, wherein the polymeric seed strand (PSS) comprises a natural or synthetic biopolymer.

20. The method of claim 19, wherein the natural biopolymer comprises a polynucleotide.

21. The method of claim 20, wherein the polynucleotide comprises a single stranded DNA (ssDNA), a partially single stranded DNA, or a double stranded DNA molecule.

22. The method of any of claims 16-21, wherein the docking linker (DL) comprises a multifunctional DL, such as a bifunctional DL or a trifunctional DL.

23. The method of claim 22, wherein the bifunctional DL comprises dibenzocyclooctyne-hexyl-N-succinimidyl (NHS) ester (DBCO-C6—NHS) having the structure:

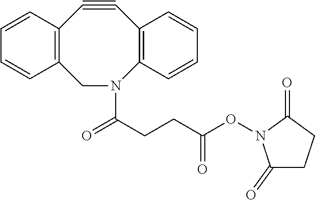 [SMILES: O=C(CCC(=O)N1Cc2ccccc2C#Cc2ccccc21)ON1C(=O)CCC1=O]

24. The method of claim 22, wherein the trifunctional DL comprises amino, DBCO, and tetra C₁-C₄ alkoxysilane groups wherein the tetra C₁-C₄ alkoxysilane group is conjugated to a solid or semisolid support.

25. The method of claim 24, wherein the trifunctional DL has the structure: 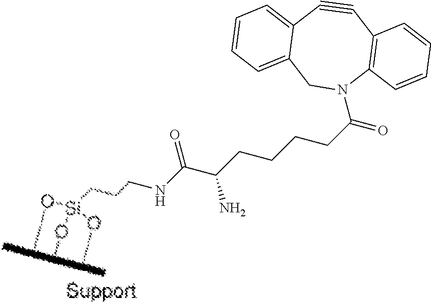 [SMILES: CNC(=O)[C@@H](N)CCCCC(=O)N1Cc2ccccc2C#Cc2ccccc21]
wherein the symbol “ [symbol] ” indicates a solid or semisolid support.

26. The method of any of claims 16-25, wherein the assembly block (AB) comprises a polynucleotide having an isothiocyanate (—N═C═S) (ITC) or isoselenocyanate (—N—C═Se) (ISC) reactive functionality for reacting with the amino group of NTAA of the polypeptide.

27. The method of any of claims 16-26, wherein ligating the AB to the proximal end of the PSS comprises an enzymatic or a non-enzymatic chemical ligation.

28. The method of claim 27, wherein enzymatic ligation comprises, stick end ligation, blunt end ligation, splint ligation, or single-strand ligation.

29. The method of claim 27, wherein the non-enzymatic chemical ligation comprises click chemistry.

30. The method of any of claims 16-29, wherein cleaving the bond between the NTAA that is attached to the AB and the prior amino acid attached to the NTAA to expose a new NTAA takes comprises Edman degradation, Edman degradation enzyme reaction, or a similar process.

31. A hybrid polymer made by the method of any of claims 15-30.

32. A method of sequencing a donor polypeptide, comprising:
preparing a hybrid polymer according to the method of any of claims 15-30; and
analyzing the hybrid polymer.

33. The method of claim 32, wherein the analyzing comprises identifying two or more amino acids of the donor polypeptide in order along the hybrid polymer.

34. The method of claim 32 or claim 33, wherein analyzing the hybrid polymer comprises passing the hybrid polymer through a nanopore to sequentially identify single amino acids of the donor polypeptide, thereby sequencing the polypeptide.

35. The method of claim 34, wherein spacing of the amino acids along the hybrid polymer is sufficient to reduce, enhance, or otherwise alter electrical signal contribution of adjacent amino acids, thereby allowing nanopore-based peptide sequencing at single-amino acid resolution.

36. The method of claim 34 or claim 35, wherein the hybrid polymer is a polynucleotide, and wherein spacing of the amino acids along the hybrid polymer is sufficient to allow a helicase enzyme to slow down the rate and movement step size of the hybrid polymer through the nanopore.

37. The method of claim 32, wherein analyzing the hybrid polymer comprises single molecule real-time (SMRT) sequencing.

38. A kit for performing the method of any of claims 15-30 or 33-37, comprising:
one or more polymeric seed strand (PSS), each having a distal end and a proximal end; and
one or more assembly blocks, conjugation reagents, ligation reagents, Edman cleavage reagents, wash buffers, solid supports, listings of barcodes, and/or analysis software.

39. A method of sequencing a polypeptide, comprising:
expanding distance between each amino acid of the polypeptide by attaching each amino acid in order to a (non-protein) polymer molecule to produce a hybrid polymer; and
analyzing the hybrid polymer, for instance by passing the hybrid polymer through a nanopore to read a single amino acid at a time or by SMRT sequencing.

40. The method of claim 39, wherein the polymer molecule comprises a nucleic acid backbone.

41. The method of claim 40, wherein the nucleic acid backbone is single stranded, partially double stranded, or double stranded.

42. The method of claim 41, wherein the amino acids of the polypeptide are attached to the polymer molecule:
from the N-terminal end to the C-terminal end of the polypeptide; or
from the C-terminal end to the N-terminal end of the polypeptide.

43. A method of sequencing a polypeptide, wherein the polypeptide has a C-terminal amino acid (CTAA) and an N-terminal amino acid (NTAA), the method comprising:
producing a hybrid polymer by:
(a) attaching a flexible linker to the CTAA of the polypeptide;
(b) attaching an (isothiocyanate (ITC) or an analogue thereof)-DNA conjugate to the NTAA of the polypeptide;
(c) aligning the end of the linker proximal to the NTAA and proximal to the end of the (ITC or an analogue thereof)-DNA conjugate though a bridge oligomer;
(d) ligating the linker proximal to the NTAA and the end of the (ITC or an analogue thereof)-DNA;
(e) cleaving the NTAA;
(f) attaching an (ITC or an analogue thereof)-DNA conjugate to the new NTAA of the polypeptide; and
(g) repeating steps (c) through (e) until the end of polypeptide is reached and there is no new NTAA left from the original polypeptide for attaching to an (ITC or an analogue thereof)-DNA conjugate; to generate a linearized and expanded polypeptide chain ready for sequencing; and
analyzing the hybrid polymer to identify each amino acid of the donor peptide, thereby sequencing the polypeptide.


SEQUENCE LISTING (87 sequences)

SEQ ID NO: 1 - DNA, 96 bp, synthetic construct
  source 1..96: mol_type = other DNA
agctttttgtttttgttgttttttttgttttttttgtttttgtttttgtttttgtttttgtttttgtttttgtgtttttgtttttgtttttgtcta

SEQ ID NO: 2 - DNA, 96 bp, synthetic construct
  source 1..96: mol_type = other DNA
agacaaaaacaaaaacaaaaacacaaaaacaaaaacaaaaacaaaaacaaaaacaaaaacaaaaaaaacaaaaaaaacaacaaaaacaaaaagcta

SEQ ID NO: 3 - DNA, 96 bp, synthetic construct
  source 1..96: mol_type = other DNA
tcctttttctttttcttcttttttttcttttttttctttttctttttctttttctttttctttttctttttctctttttctttttctttttctctt

SEQ ID NO: 4 - DNA, 21 bp, synthetic construct
  source 1..21: mol_type = other DNA
  modified_base 1: mod_base = OTHER; note = 5 prime dibenzocyclooctyne
  modified_base 2: mod_base = OTHER; note = biotin modified thymine
  misc_feature 21: note = 2-nitrobenzyl photocleavable linker
ttttttttttttttttttttt

SEQ ID NO: 5 - DNA, 36 bp, synthetic construct
  source 1..36: mol_type = other DNA
  modified_base 1: mod_base = OTHER; note = 5 prime phopshorylation
  modified_base 18: mod_base = OTHER; note = hexyl-amine modified thymine
ctctcctccttctctttttttctcttctccctctcc

SEQ ID NO: 6 - DNA, 36 bp, synthetic construct
  source 1..36: mol_type = other DNA
  modified_base 1: mod_base = OTHER; note = 5 prime phosphorylation
  modified_base 18: mod_base = OTHER; note = hexyl-amine modified thymine
ttttcctctccttctttttttctcctctctcctcct

SEQ ID NO: 7 - DNA, 42 bp, synthetic construct
  source 1..42: mol_type = other DNA
  modified_base 1: mod_base = OTHER; note = 5 prime phosphorylation
ctctcctccttctcttvyyyyyvtagatcggaagagcgtcgt

SEQ ID NO: 8 - DNA, 30 bp, synthetic construct
  source 1..30: mol_type = other DNA
agagaaggaggagagaggaggagagaggag

SEQ ID NO: 9 - DNA, 30 bp, synthetic construct
  source 1..30: mol_type = other DNA
agaaggagaggaaaaggagagggagaagag

SEQ ID NO: 10 - DNA, 36 bp, synthetic construct; identical to SEQ ID NO: 5
  source 1..36: mol_type = other DNA
  modified_base 1: mod_base = OTHER; note = 5 prime phosphorylation
  modified_base 18: mod_base = OTHER; note = ethyl-amine modified thymine

SEQ ID NO: 11 - DNA, 36 bp, synthetic construct; identical to SEQ ID NO: 6
  source 1..36: mol_type = other DNA
  modified_base 1: mod_base = OTHER; note = 5 prime phosphorylation
  modified_base 18: mod_base = OTHER; note = ethyl-amine modified thymine

SEQ ID NO: 12 - DNA, 17 bp, synthetic construct
  source 1..17: mol_type = other DNA
  modified_base 1: mod_base = OTHER; note = 5 prime phosphorylation
  misc_feature 17: note = Uni-Link Amino Modifier
ctctcctccttctcttt

SEQ ID NO: 13 - DNA, 36 bp, synthetic construct; identical to SEQ ID NO: 5
  source 1..36: mol_type = other DNA
  modified_base 1: mod_base = OTHER; note = 5 prime phosphorylation
  modified_base 18: mod_base = OTHER; note = 5-Octadiynyl dU

SEQ ID NO: 14 - DNA, 24 bp, synthetic construct
  source 1..24: mol_type = other DNA
ggagagaggaggagagaggagaaa

SEQ ID NO: 15 - DNA, 23 bp, synthetic construct
  source 1..23: mol_type = other DNA
aggaaaaggagagggagaagaga

SEQ ID NO: 16 - DNA, 47 bp, synthetic construct
  source 1..47: mol_type = other DNA
  modified_base 1: mod_base = OTHER; note = 5 prime phosphorylation
ctctcctccttctcttatgcgatcggaatagatcggaagagcgtcgt

SEQ ID NO: 17 - DNA, 47 bp, synthetic construct
  source 1..47: mol_type = other DNA
  modified_base 1: mod_base = OTHER; note = 5 prime phosphorylation
ctctcctccttctcttccggattagacgtagatcggaagagcgtcgt

SEQ ID NO: 18 - DNA, 47 bp, synthetic construct
  source 1..47: mol_type = other DNA
  modified_base 1: mod_base = OTHER; note = 5 prime phosphorylation
ctctcctccttctcttcgtcatagtggatagatcggaagagcgtcgt

SEQ ID NO: 19 - DNA, 47 bp, synthetic construct
  source 1..47: mol_type = other DNA
  modified_base 1: mod_base = OTHER; note = 5 prime phosphorylation
ctctcctccttctcttgagataggatgttagatcggaagagcgtcgt

SEQ ID NO: 20 - DNA, 47 bp, synthetic construct
  source 1..47: mol_type = other DNA
  modified_base 1: mod_base = OTHER; note = 5 prime phosphorylation
ctctcctccttctcttggcagcaatccatagatcggaagagcgtcgt

SEQ ID NO: 21 - DNA, 47 bp, synthetic construct
  source 1..47: mol_type = other DNA
  modified_base 1: mod_base = OTHER; note = 5 prime phosphorylation
ctctcctccttctcttgctataatagtgtagatcggaagagcgtcgt

SEQ ID NO: 22 - DNA, 47 bp, synthetic construct
  source 1..47: mol_type = other DNA
  modified_base 1: mod_base = OTHER; note = 5 prime phosphorylation
ctctcctccttctcttatcagtcagagttagatcggaagagcgtcgt

SEQ ID NO: 23 - DNA, 47 bp, synthetic construct
  source 1..47: mol_type = other DNA
  modified_base 1: mod_base = OTHER; note = 5 prime phosphorylation
ctctcctccttctcttaccgttacgccatagatcggaagagcgtcgt

SEQ ID NO: 24 - DNA, 47 bp, synthetic construct
  source 1..47: mol_type = other DNA
  modified_base 1: mod_base = OTHER; note = 5 prime phosphorylation
ctctcctccttctcttccgaacggcgcgtagatcggaagagcgtcgt

SEQ ID NO: 25 - DNA, 47 bp, synthetic construct
  source 1..47: mol_type = other DNA
  modified_base 1: mod_base = OTHER; note = 5 prime phosphorylation
ctctcctccttctcttatcatcagaggatagatcggaagagcgtcgt

SEQ ID NO: 26 - protein, 12 aa, synthetic construct
  MOD_RES 12: note = azido group attached to lysine at C terminus
WGSGGSGGSDDK

SEQ ID NO: 27 - protein, 9 aa, synthetic construct
WGGSGGSDC

SEQ ID NO: 28 - protein, 9 aa, synthetic construct
GGGSGGSDC

SEQ ID NO: 29 - protein, 9 aa, synthetic construct
RGGSGGSDC

SEQ ID NO: 30 - protein, 9 aa, synthetic construct
HGGSGGSDC

SEQ ID NO: 31 - DNA, 110 bp, synthetic construct
  source 1..110: mol_type = other DNA
tttttcccctccttctttttctctcctctctcctcctctctcctccttctcaaaaggcttctcttctccctctccttttcctctccttctttcctcctctctcctcctct

SEQ ID NO: 32 - DNA, 110 bp, synthetic construct
  source 1..110: mol_type = other DNA
tttttcccctccttctttttctcctctctcctcctctctcctccttctcttgttcctcttctccctctccttttcctctccttcttttagttctcctctctcctcctctc

SEQ ID NO: 33 - DNA, 110 bp, synthetic construct
  source 1..110: mol_type = other DNA
tttttcccctccttctttttctcctctctcctcctctctcctccttctcaaggctttctcttctccctctccttttcctctccttctttttctgacctcctctctcctcc

SEQ ID NO: 34 - DNA, 110 bp, synthetic construct
  source 1..110: mol_type = other DNA
tttttcccctccttctttttctcctctctcctcctctctcctccttctcgtgtttctcttctccctctccttttcctctccttcggggcctcctctctcctcctctctcc

SEQ ID NO: 35 - DNA, 110 bp, synthetic construct
  source 1..110: mol_type = other DNA
tttttcccctccttctttttttctcctctctcctcctctctcctccttctcaaggcttctcttctccctctccttttcctctccttcttctcctctctcctcctctctcc

SEQ ID NO: 36 - DNA, 110 bp, synthetic construct
  source 1..110: mol_type = other DNA
tttttcccctccttctttttctcctctctcctcctctctcctccttctctttcctcttctccctctccttttcctctccttcttttttctcctctctcctcctctctcct

SEQ ID NO: 37 - DNA, 110 bp, synthetic construct
  source 1..110: mol_type = other DNA
tttttcccctccttcttttttctcctctctcctcctctctcctccttctcgtttctcttctccctctccttttcctctccttcagctttctcctctctcctcctctctcc

SEQ ID NO: 38 - DNA, 110 bp, synthetic construct
  source 1..110: mol_type = other DNA
tttttcccctccttcttttttctcctctctcctcctctctcctccttctcaggcttcctcttctccctctccttttcctctccttcagtttctcctctctcctcctctct

SEQ ID NO: 39 - DNA, 110 bp, synthetic construct
  source 1..110: mol_type = other DNA
tttttcccctccttcttttttctcctctctcctcctctctcctccttctcttgttctcttctccctctccttttcctctccttcgccggacttctcctctctcctcctct

SEQ ID NO: 40 - DNA, 110 bp, synthetic construct
  source 1..110: mol_type = other DNA
tttttcccctccttcttttttttctcctctctcctcctctctcctccttctcaggtttctcttctccctctccttttcctctccttctttttctcctctctcctcctctc

SEQ ID NO: 41 - DNA, 110 bp, synthetic construct
  source 1..110: mol_type = other DNA
tttttcccctccttcttttttctcctctctcctcctctctcctccttctcatttctcttctccctctccttttcctctccttcttgaacctcctctctcctcctctctcc

SEQ ID NO: 42 - DNA, 110 bp, synthetic construct
  source 1..110: mol_type = other DNA
tttttcccctccttcttttttctcctctctcctcctctctcctccttctcttttttctcttctccctctccttttcctctccttcttttttctcctctctcctcctctct

SEQ ID NO: 43 - DNA, 110 bp, synthetic construct
  source 1..110: mol_type = other DNA
tttttcccctccttctttttttctcctctctcctcctctctcctccttctctttgtttctcttctccctctccttttcctctccttcttcgttctcctctctcctcctct

SEQ ID NO: 44 - DNA, 110 bp, synthetic construct
  source 1..110: mol_type = other DNA
tttttcccctccttcttttttctcctctctcctcctctctcctccttctcttgttttctcttctccctctccttttcctctccttctttgtccgttctcctctctcctcc

SEQ ID NO: 45 - DNA, 110 bp, synthetic construct
  source 1..110: mol_type = other DNA
tttttcccctccttctttttctcctctctcctcctctctcctccttctcagagcctcttctccctctccttttcctctccttcttgttttctcctctctcctcctctctc

SEQ ID NO: 46 - DNA, 110 bp, synthetic construct
  source 1..110: mol_type = other DNA
tttttcccctccttcttttttctcctctctcctcctctctcctccttctcaaggcttcctcttctccctctccttttcctctccttcaagtttcctcctctctcctcctc

SEQ ID NO: 47 - DNA, 110 bp, synthetic construct
  source 1..110: mol_type = other DNA
tttttcccctccttctttttttctcctctctcctcctctctcctccttctcttcgttctcttctccctctccttttcctctccttcttcgttttctcctctctcctcctc

SEQ ID NO: 48 - DNA, 110 bp, synthetic construct
  source 1..110: mol_type = other DNA
tttttcccctccttcttttttctcctctctcctcctctctcctccttctcttgtttcctcttctccctctccttttcctctccttctttctggttctcctctctcctcct

SEQ ID NO: 49 - DNA, 110 bp, synthetic construct
  source 1..110: mol_type = other DNA
tttttcccctccttctttttttctcctctctcctcctctctcctccttctcgtttctcttctccctctccttttcctctccttcttggttctcctctctcctcctctctc

SEQ ID NO: 50 - DNA, 110 bp, synthetic construct
  source 1..110: mol_type = other DNA
tttttcccctccttcttttttctcctctctcctcctctctcctccttctcttctttgttctcttctccctctccttttcctctccttctttggtttctcctctctcctcc

SEQ ID NO: 51 - DNA, 110 bp, synthetic construct
  source 1..110: mol_type = other DNA
tttttcccctccttctttttctcctctctcctcctctctcctccttctcaggctttctcttctccctctccttttcctctccttcatgagactttctcctctctcctcct

SEQ ID NO: 52 - DNA, 110 bp, synthetic construct
  source 1..110: mol_type = other DNA
tttttcccctccttctttttctcctctctcctcctctctcctccttctctttgccgtttcctcttctccctctccttttcctctccttctttcgttcctcctctctcctc

SEQ ID NO: 53 - DNA, 110 bp, synthetic construct
  source 1..110: mol_type = other DNA
tttttcccctccttctttttttctcctctctcctcctctctcctccttctcaaggaactttctcttctccctctccttttcctctccttcttgttctcctctctcctcct

SEQ ID NO: 54 - DNA, 110 bp, synthetic construct
  source 1..110: mol_type = other DNA
tttttcccctccttctttttttctcctctctcctcctctctcctccttctcttgttctcttctccctctccttttcctctccttctttcgttctcctctctcctcctctc

SEQ ID NO: 55 - DNA, 110 bp, synthetic construct
  source 1..110: mol_type = other DNA
tttttcccctccttctttttctcctctctcctcctctctcctccttctcttcctccttctcttttttcctcttctccctctccttttcctctccttcttttttctcctct

SEQ ID NO: 56 - DNA, 21 bp, synthetic construct
  source 1..21: mol_type = other DNA
  modified_base 1: mod_base = OTHER; note = 5 prime dibenzocyclooctyne
  modified_base 2: mod_base = OTHER; note = biotin modified thymine
  misc_feature 21: note = 2-nitrophenyl photocleavable linker
ttttttttttttttttttttt

SEQ ID NO: 57 - DNA, 36 bp, synthetic construct; identical to SEQ ID NO: 5
  source 1..36: mol_type = other DNA
  modified_base 1: mod_base = OTHER; note = 5 prime phosphorylation
  misc_feature 18: note = linker (e.g., amine, isothiocyanate, isoselenocyanate, click reagent)

SEQ ID NO: 58 - protein, 19 aa, synthetic construct
  MOD_RES 19: note = azido group attached to lysine
RGGGRGRGGRGGGSGGGSK

SEQ ID NO: 59 - DNA, 36 bp, synthetic construct; identical to SEQ ID NO: 6
  source 1..36: mol_type = other DNA
  modified_base 1: mod_base = OTHER; note = 5 prime phosphorylation
  misc_feature 18: note = linker (e.g., amine, isothiocyanate, isoselenocyanate, click reagent)

SEQ ID NO: 60 - protein, 19 aa, synthetic construct
  MOD_RES 19: note = azido group attached to lysine
GGRGRGRGGRGGGSGGGSK

SEQ ID NO: 61 - DNA, 42 bp, synthetic construct
  source 1..42: mol_type = other DNA
  modified_base 1: mod_base = OTHER; note = 5 prime phosphorylation
  misc_feature 16..24: note = unique molecular identifier
ctctcctccttctcttvnnnnnvtagatcggaagagcgtcgt

SEQ ID NO: 62 - DNA, 36 bp, synthetic construct
  source 1..36: mol_type = other DNA
  misc_feature 1: note = 2-nitrobenzyl photocleavable linker
ttttcccctccttctttttttctcctctctcctcct

SEQ ID NO: 63 - DNA, 18 bp, synthetic construct
  source 1..18: mol_type = other DNA
  misc_feature 1: note = Uni-Link Amino Modifier
tttctcttctccctctcc

SEQ ID NO: 64 - DNA, 36 bp, synthetic construct; identical to SEQ ID NO: 62
  source 1..36: mol_type = other DNA
  misc_feature 1: note = 2-nitrophenyl photocleavable linker

SEQ ID NO: 65 - protein, 19 aa, synthetic construct
  MOD_RES 19: note = azido group attached to lysine
WGRGRGRGGRGGGSGGGSK

SEQ ID NO: 66 - protein, 19 aa, synthetic construct
  MOD_RES 19: note = azido group attached to lysine
LGRGRGRGGRGGGSGGGSK

SEQ ID NO: 67 - protein, 23 aa, synthetic construct
  MOD_RES 23: note = azido group attached to lysine
LWAEFGRGRGRGGRGGGSGGGSK

SEQ ID NO: 68 - DNA, 21 bp, synthetic construct
  source 1..21: mol_type = other DNA
agagaaggaggagagaggagg

SEQ ID NO: 69 - DNA, 17 bp, synthetic construct
  source 1..17: mol_type = other DNA
  misc_feature 1: note = 5 prime photocleavable biotin
  misc_feature 17: note = C3 phosphoramidite spacer
ttttcctctccttcttt

SEQ ID NO: 70 - DNA, 18 bp, synthetic construct
  source 1..18: mol_type = other DNA
  misc_feature 1: note = C3 phosphoramidite spacer
tttctcctctctcctcct

SEQ ID NO: 71 - DNA, 17 bp, synthetic construct
  source 1..17: mol_type = other DNA
  misc_feature 17: note = C3 phosphoramidite spacer
  modified_base 1: mod_base = OTHER; note = 5 prime phosphorylation
ttttcctctccttcttt

SEQ ID NO: 72 - DNA, 33 bp, synthetic construct
  source 1..33: mol_type = other DNA
  misc_feature 1: note = C3 phosphoramidite spacer
tttgtgtagttccacagatcggaagagcgtcgt

SEQ ID NO: 73 - DNA, 33 bp, synthetic construct
  source 1..33: mol_type = other DNA
  misc_feature 1: note = C3 phosphoramidite spacer
tttaggctcataactagatcggaagagcgtcgt

SEQ ID NO: 74 - DNA, 33 bp, synthetic construct
  source 1..33: mol_type = other DNA
  misc_feature 1: note = C3 phosphoramidite spacer
tttgatatgtctacgagatcggaagagcgtcgt

SEQ ID NO: 75 - DNA, 33 bp, synthetic construct
  source 1..33: mol_type = other DNA
  misc_feature 1: note = C3 phosphoramidite spacer
ttttcctaactgaggagatcggaagagcgtcgt

SEQ ID NO: 76 - DNA, 33 bp, synthetic construct
  source 1..33: mol_type = other DNA
  misc_feature 1: note = C3 phosphoramidite spacer
tttatctatggcataagatcggaagagcgtcgt

SEQ ID NO: 77 - DNA, 33 bp, synthetic construct
  source 1..33: mol_type = other DNA
  misc_feature 1: note = C3 phosphoramidite spacer
tttatacctgttggcagatcggaagagcgtcgt

SEQ ID NO: 78 - DNA, 33 bp, synthetic construct
  source 1..33: mol_type = other DNA
  misc_feature 1: note = C3 phosphoramidite spacer
tttttaagtcgcgccagatcggaagagcgtcgt

SEQ ID NO: 79 - DNA, 33 bp, synthetic construct
  source 1..33: mol_type = other DNA
  misc_feature 1: note = C3 phosphoramidite spacer
tttccagactaagacagatcggaagagcgtcgt

SEQ ID NO: 80 - DNA, 33 bp, synthetic construct
  source 1..33: mol_type = other DNA
  misc_feature 1: note = C3 phosphoramidite spacer
ttttaaggtcgcaatagatcggaagagcgtcgt

SEQ ID NO: 81 - DNA, 33 bp, synthetic construct
  source 1..33: mol_type = other DNA
  misc_feature 1: note = C3 phosphoramidite spacer
tttccgtggatctagagatcggaagagcgtcgt

SEQ ID NO: 82 - DNA, 33 bp, synthetic construct
  source 1..33: mol_type = other DNA
  misc_feature 1: note = C3 phosphoramidite spacer
tttacaagatcaatcagatcggaagagcgtcgt

SEQ ID NO: 83 - DNA, 33 bp, synthetic construct
  source 1..33: mol_type = other DNA
  misc_feature 1: note = C3 phosphoramidite spacer
tttccacgatcacgaagatcggaagagcgtcgt

SEQ ID NO: 84 - DNA, 22 bp, synthetic construct
  source 1..22: mol_type = other DNA
ctccacgacgctcttccgatct

SEQ ID NO: 85 - DNA, 483 bp, synthetic construct
  source 1..483: mol_type = other DNA
ggagaggtcttcggagagacggaagaggtgtagaagcctaggaacaggttagtatgagtagcttaagaatgtaaattctgtgattatagtgtagtaatctctaattaacggtgacgcaaaatcaagcgcagtgatttcaacagataatgctgatggtttaggcgtacaatgcgctgaagaataattaagaaaatagcactcctcgtcgcctagaattacctaccggcgtccaccataccttcgattatcgcgcccactctcccattagtcggcacaggtggatgtgttgcgatagcccgctaagatattctaaggcgtaacgcagatgaatattctacagagttgccataggcgttgaacgcttcacggacgataggaattagcgtatagagcgcgtcatcgaagagttatacactcgtagttaacatctagcccggctatgcgatcggaatcgggtgagacctctcctttccttctcctttc

SEQ ID NO: 86 - DNA, 483 bp, synthetic construct
  source 1..483: mol_type = other DNA
ggagaggtcttcggagagacggaagaggtgtagaagcctaggaacaggttagtatgagtagcttaagaatgtaaattctgtgattatagtgtagtaatctctaattaacggtgacgcaaaatcaagcgcagtgatttcaacagataatgctgatggtttaggcgtacaatgcgctgaagaataattaagaaaatagcactcctcgtcgcctagaattacctaccggcgtccaccataccttcgattatcgcgcccactctcccattagtcggcacaggtggatgtgttgcgatagcccgctaagatattctaaggcgtaacgcagatgaatattctacagagttgccataggcgttgaacgcttcacggacgataggaattagcgtatagagcgcgtcatcgaagagttatacactcgtagttaacatctagcccggctccggattagacgtcgggtgagacctctcctttccttctcctttc

SEQ ID NO: 87 - DNA, 36 bp, synthetic construct
  source 1..36: mol_type = other DNA
ggaggagagaggagaaaaaaagaaggagaggaaaaa